(19) United States
(12) Patent Application Publication
(10) Pub. No.: US 2023/0220387 A1
(43) Pub. Date: Jul. 13, 2023

(54) D-SERINE TRANSPORT MODIFIER AND SCREENING METHOD THEREOF, AND SCREENING METHOD OF D-SERINE TRANSPORTER PROTEIN

(71) Applicants: Kagami Inc., Ibaraki-shi, Osaka (JP); Public University Corporation Nara Medical University, Kashihara-shi, Nara (JP); National Institutes of Biomedical Innovation, Health and Nutrition, Ibaraki-shi, Osaka (JP)

(72) Inventors: Masashi MITA, Osaka (JP); Rikako SUZUKI, Tokyo (JP); Shushi NAGAMORI, Nara (JP); Pattama WIRIYASERMKUL, Nara (JP); Pornparn KONGPRACHA, Nara (JP); Satomi MORIYAMA, Nara (JP); Tomonori KIMURA, Osaka (JP)

(73) Assignees: Kagami Inc., Ibaraki-shi, Osaka (JP); Public University Corporation Nara Medical University, Kashihara-shi, Nara (JP); National Institutes of Biomedical Innovation, Health and Nutrition, Ibaraki-shi, Osaka (JP)

(21) Appl. No.: 17/789,399

(22) PCT Filed: Dec. 25, 2020
(86) PCT No.: PCT/JP2020/049031

§ 371 Date: Jun. 27, 2022

(30) Foreign Application Priority Data
Dec. 27, 2019 (JP) 2019-239834

(51) Int. Cl. C12N 15/113; A61K 31/192; A61K 31/616; A61K 31/465; A61K 31/198; A61K 31/197

(52) U.S. Cl. CPC C12N 15/113; A61K 31/192; A61K 31/616; A61K 31/465; A61K 31/198; A61K 31/197; C12N 2310/14

(57) ABSTRACT

The present invention provides a D-serine transport modifier which is characterized by controlling the transport of D-serine into and out of cells by a D-serine transporter protein, a pharmaceutical composition which comprises the same as an active component and treats or prevents diseases relating to an increase or decrease in the amount of D-serine, and a screening method of substances that control the transport of D-serine. The present invention also provides a screening method of a D-serine transporter protein.

### CROSS-REFERENCE TO RELATED APPLICATIONS

This application is the U.S. National Stage of PCT/JP2020/049031, filed Dec. 25, 2010, which claims priority to JP 2019-239834, filed Dec. 27, 2019.

The instant application contains a Sequence Listing which has been submitted in ASCII format via EFS-WEB and is hereby incorporated by reference in its entirety. Said ASCII copy, created on Jun. 27, 2022, is named sequence.txt and is 210,827 bytes.

### FIELD

The present invention relates to a D-serine transport modulator, a pharmaceutical composition for the treatment or prevention of a disease associated with an increase or a decrease in a D-serine level, a method for treating or preventing a disease associated with an increase or a decrease in a D-serine level, and a method of screening for a substance which modulates D-serine transport. The present invention also relates to a method of screening for a D-serine transport protein.

### BACKGROUND

D-amino acids, which were previously thought to be absent in the mammalian body, have been shown to be present in various tissues and to play various physiological functions. D-serine, a D-amino acid, has been shown to be a potential biomarker reflecting kidney functions and kidney diseases (Non-Patent Literature documents 1 to 3).

However, in the process of developing D-serine as a marker for kidney diseases, the transport system of D-serine in the kidney was not clear. To understand the physiological significance of D-serine, it is necessary, and strongly desired, to identify the transporter molecules involved in the transport mechanism of D-serine into and out of cells.

ASCT1 (SLC1A4) (Non-Patent Literature documents 4 and 5), ASCT2 (SLC1A5) (Non-Patent Literature document 5), Asc1 (SLC7A10) (Non-Patent Literature documents 6 and 7), PAT1 (SLC36A1) (Non-Patent Literature document 8), and ATB^{0,+} (SLC6A14) (Non-Patent Literature document 9) are known to play the role of membrane transport proteins of D-serine. However, it is not clear whether these proteins play the role of membrane transport proteins of D-serine in the kidney.

### CITATION LIST

### Non Patent Literature

[Non-Patent Literature 1] Sasabe J., et al., Ischemic acute kidney injury perturbs homeostasis of serine enantiomers in the body fluid in mice: early detection of renal dysfunction using the ratio of serine enantiomers. PLoS One. 2014 Jan. 29; 9(1):e86504.
[Non-Patent Literature 2] Silbernagl S., et al., D-Serine is reabsorbed in rat renal pars recta. Am J Physiol. 1999 June; 276(6):F857-63.
[Non-Patent Literature 3] Hesaka A., et al., D-Serine reflects kidney function and diseases. Sci Rep. 2019 Mar. 25; 9(1):5104.
[Non-Patent Literature 4] Kaplan E. et al., ASCT1 (Slc1a4) transporter is a physiologic regulator of brain d-serine and neurodevelopment. Proc Natl Acad Sci USA. 2018 Sep. 18; 115(38):9628-9633.
[Non-Patent Literature 5] Foster A C., et al., D-Serine Is a Substrate for Neutral Amino Acid Transporters ASCT1/SLC1A4 and ASCT2/SLC1A5, and Is Transported by Both Subtypes in Rat Hippocampal Astrocyte Cultures. PLoS One. 2016 Jun. 7; 11(6):e0156551.
[Non-Patent Literature 6] Kasai Y., et al., Transport systems of serine at the brain barriers and in brain parenchymal cells. J Neurochem. 2011 July; 118(2):304-13.
[Non-Patent Literature 7] Rosenberg D., et al., Neuronal D-serine and glycine release via the Asc-1 transporter regulates NMDA receptor-dependent synaptic activity. J Neurosci. 2013 Feb. 20; 33(8):3533-44.
[Non-Patent Literature 8] Chen Z., et al., Structure, function and immunolocalization of a proton-coupled amino acid transporter (hPAT1) in the human intestinal cell line Caco-2. J Physiol. 2003 Jan. 15; 546(Pt 2):349-61.
[Non-Patent Literature 9] Hatanaka T., et al., Transport of D-serine via the amino acid transporter ATB(0,+) expressed in the colon. Biochem Biophys Res Commun. 2002 Feb. 22; 291(2):291-5.

### SUMMARY

### Technical Problem

It is desirable to develop a drug for treating or preventing diseases associated with elevated or decreased D-serine levels, e.g., kidney diseases. To this end, it is necessary to identify novel D-serine transport proteins that can regulate the amount of D-serine in cells, tissues, organs, or body fluids. A method of screening for such proteins are therefore sought after.

### Solution to Problem

After intensive research, the present inventors have identified D-serine transport proteins that can regulate the amount of D-serine in cells, tissues, organs, or body fluids, and have found that the use of such D-serine transport proteins or substances acting on these proteins as D-serine transport regulators makes it possible to treat or prevent diseases related to increased or decreased D-serine levels (e.g., kidney diseases). The inventors have also developed a simple method of screening for novel D-serine transport proteins that can regulate the D-serine levels in cells, tissues, organs, or body fluids, as well as novel substances that act on D-serine transport proteins. Thus, the present invention encompasses the following aspects.

[1] A D-serine transport modulator that modulates intracellular and extracellular D-serine transport by a D-serine transport protein.
[2] The D-serine transport modulator according to Aspect 1, wherein the D-serine transport proteins comprises one or more selected from a first group of D-serine transport proteins consisting of SMCT family, GLUT5, CAT1, THTR2 and SNAT2.
[3] The D-serine transport modulator according to Aspect 2, wherein the D-serine transport protein further comprises one or more selected from a second group of D-serine transport proteins group consisting of ASCT family, Asc1, PAT1 and ATB^{0,+}.
[4] The D-serine transport modulator according to any one of Aspects 1 to 3, which modulates a D-serine level in a cell, in a tissue, in an organ, or in a body fluid.
[5] The D-serine transport modulator according to any one of Aspects 1 to 3, which modulates a D-serine level in blood and/or in urine.
[6] The D-serine transport modulator according to any one of Aspects 1 to 5, which inhibits D-serine transport into a cell by acting on the D-serine transport protein.
[7] The D-serine transport modulator according to Aspect 1, which is selected from the group consisting of antisense RNA or DNA molecules, RNAi inducible nucleic acids, micro RNA(miRNA), ribozymes, genome-editing nucleic acids and their expression vectors, low-molecular compounds, aptamers, antibodies, antibody fragments, and combinations thereof.
[8] The D-serine transport modulator according to Aspect 6, which is a substrate or inhibitor of the D-serine transport protein.
[9] The D-serine transport modulator according to Aspect 6, which comprises one or more selected from a first group of substrates or inhibitors of the D-serine transport protein consisting of ibuprofen, fenoprofen, ketoprofen, probenecid, acetyl salicylic acid, naproxen, pyroglutamic acid, phenoxyacetic acid, acetic acid, propionic acid, butyric acid, L-lactic acid, D-lactic acid, pyruvic acid, nicotinic acid, acetoacetic acid, β-D-hydroxybutyric acid, β-L-hydroxybutyric acid, γ-hydroxybutyric acid, α-ketoisocaproic acid, benzoic acid, salicylic acid, 5-amino salicylic acid, 2,4-dichlorophenoxyacetic acid (2,4-D), 4-chlorophenoxyacetic acid (4-CPA), 2-chlorophenoxyacetic acid (2-CPA), 2,3-dichlorophenoxyacetic acid, 3,4-dichlorophenoxyacetic acid, 2,4,5-trichlorophenoxyacetic acid, N-(4-methane sulphonyl-2-nitrophenyl)-2H-1,3-benzodioxol-5-amine (MSNBA), fructose, N-ethylmaleimide (NEM), N-amino-L-arginine, N-amino-L-homoarginine, L-arginine, L-histidine, L-lysine, L-ornithine, metformin, chloroquine, 2,4-diamino pyrimidine, Fedratinib, AZD1480, Cerdulatinib, thiamine, methyl-amino-isobutyric acid (MeAIB), γ-glutamyl-p-nitroanilide (GPNA), 2-amino-4-bis(aryloxybenzil)amino butyric acid (AABA), L-alanine, L-methionine, L-proline, L-serine, L-asparagine, L-glutamine, L-histidine, glycine and its derivatives, and pharmaceutically acceptable salts thereof.
[10] The D-serine transport modulator according to Aspect 9, which further comprises one or more selected from a second group of substrates or inhibitors of the D-serine transport protein consisting of phenylglycine analog, benzilserine, benzilcysteine, S-benzil-L-cystine, L-γ-glutamyl-p-nitroanilide, L-serine, L-threonine, L-methionine, L-alanine, L-cysteine, L-glutamine, D-alanine, phenylglycine analog, alanine analog, L-serine, L-alanine, L-cysteine, glycine, L-threonine, taurine, GABA, tryptophan, tryptamine derivative, 5-hydroxy-L-tryptophan, serotonin, indole-3-propionic acid, α-methyl-DL-tryptophan and its derivatives, and pharmaceutically acceptable salts thereof.
[11] A pharmaceutical composition for treating or preventing a disease associated with an increase in a D-serine level in a cell, in a tissue, in an organ, or in a body fluid, comprising the D-serine transport modulator according to any one of Aspects 6 to 10 as an active ingredient.
[12] The pharmaceutical composition according to Aspect 11, wherein the disease associated with an increase in the D-serine level is a kidney disease.
[13] A method for treating or preventing a disease associated with an increase in a D-serine level in a cell, in a tissue, in an organ, or in a body fluid, comprising administering the D-serine transport modulator according to any one of Aspects 6 to 10 as an active ingredient to a subject in need thereof.
[14] The method according to Aspect 13, wherein the disease associated with an increase in the D-serine level is a kidney disease.
[15] The D-serine transport modulator according to any one of Aspects 1 to 5, which enhances D-serine transport into a cell by acting on the D-serine transport protein.
[16] The D-serine transport modulator according to Aspect 15, which is selected from the group consisting of vectors expressing the D-serine transport protein, its derivative, or a part thereof, low-molecular compounds, aptamers, antibodies, antibody fragments, and combinations thereof.
[17] The D-serine transport modulator according to Aspect 15, which is selected from the group consisting of diclofenac, curcumin, activin A, and SMCT family-, GLUT5-, CAT1-, THTR2-, SNAT2- and PDZK1-expression vectors.
[18] A pharmaceutical composition for treating or preventing a disease associated with a decrease in a D-serine level in a cell, in a tissue, in an organ, or in a body fluid, comprising the D-serine transport modulator according to any one of Aspects 15 to 17 as an active ingredient.
[19] The pharmaceutical composition according to Aspect 18, wherein the disease associated with a decrease in a D-serine level is a kidney disease.
[20] A method for treating or preventing a disease associated with a decrease in a D-serine level in a cell, in a tissue, in an organ, or in a body fluid, comprising administering the D-serine transport modulator according to any one of Aspects 15 to 17 as an active ingredient to a subject in need thereof.
[21] The method according to Aspect 20, wherein the disease associated with a decrease in a D-serine level is a kidney disease.
[22] A method of screening for a substance that modulates intracellular and extracellular D-serine transport by a D-serine transport protein, comprising:

applying a candidate substance and D-serine to a cell expressing a D-serine transport protein; and

evaluating the degree of intracellular and extracellular D-serine transport based on the expression of cytotoxicity as an indicator.

[23] The method according to Aspect 22, wherein the D-serine transport protein comprises one or more selected from a first group of D-serine transport proteins consisting of SMCT family, GLUT5, CAT1, THTR2 and SNAT2.
[24] The method according to Aspect 23, wherein the D-serine transport protein further comprises one or more selected from a second D-serine transport protein group consisting of ASCT family, Asc1, PAT1 and ATB^{0,+}.
[25] The method according to any one of Aspects 22 to 24, wherein the cell is a cell engineered by externally introducing a vector expressing the D-serine transport protein.
[26] The method according to any one of Aspects 22 to 24, comprising selecting substances which inhibit D-serine transport into a cell by acting on the D-serine transport protein to screen for the effect of treating or preventing a disease associated with an increase in the D-serine level in a cell, in a tissue, in an organ, or in a body fluid.
[27] The method according to Aspect 26, wherein the disease associated with an increase in the D-serine level is a kidney disease.
[28] The method according to any one of Aspects 22 to 24, comprising selecting substances which enhance D-serine transport into a cell by acting on the D-serine transport protein to screen for the effect of treating or preventing a disease associated with a decrease in a D-serine level in a cell, in a tissue, in an organ, or in a body fluid.
[29] The method according to Aspect 28, wherein the disease associated with a decrease in a D-serine level is a kidney disease.
[30] A method of screening a D-serine transport protein based on D-serine transport into a cell as an indicator.
[31] The method according to Aspect 30, wherein the expression of cytotoxicity produced by the addition of D-serine is used as an indicator of the D-serine transport into a cell.
[32] The method according to Aspect 30 or 31, wherein the cell has been engineered to express a candidate transport protein.
[33] The method according to Aspect 32, wherein the cell has been engineered by externally introducing a vector expressing the D-serine transport protein.
[33] The method according to Aspect 33, wherein the vector is selected from the group consisting of plasmid vectors, cosmid vectors, fosmid vectors, artificial chromosome vectors, and virus vectors.

### Advantageous Effects of Invention

The present invention makes it possible to modulate the D-serine levels in cells, tissues, organs, or body fluids, and to thereby treat or prevent diseases associated with increased or decreased D-serine levels, such as kidney diseases. The present invention also makes it possible to develop novel D-serine transport proteins that can modulate the D-serine levels in cells, tissues, organs, or body fluids, and novel D-serine transport proteins that act on D-serine transport proteins.

### BRIEF DESCRIPTION OF DRAWINGS

FIG. 1 shows that D-serine transport in mouse brush border membrane vesicles (BBMV) is primarily Na⁺-dependent transport. The time-dependent change in 10 μM (A) or 50 μM (B) D-[³H]serine uptake activity in BBMV was measured in the presence of Na⁺ (Na⁺) or in its absence (K⁺). *P<0.05.

FIG. 2 shows that D-serine transport in BBMV was inhibited mainly by ASCT2 and SMCT inhibitors. Shown are the results of measuring D-[³H]serine transport (10 μM) activity in the presence or in the absence (−) of 1 mM nicotinic acid or 2 mM L-threonine (L-Thr). *P<0.05, NS: No significant difference.

FIG. 3 shows that human SMCT1 and hSMCT2 transport D-serine. An FlpIn293TR-hSLC5A8-3×FLAG (HSMCT1) or FlpIn293TR-hSLC5A12-3×FLAG (hSMCT2)-stable cell line was prepared, and (A) after ASCT2siRNA knockdown of Flp-In TREx 293 cells, expression was confirmed by Western blotting using anti-ASCT2 antibody. (B) Two days before the uptake experiment, doxycycline was added to the hSMCT1 and SMCT2 cells to induce expression. Expression was confirmed by Western blotting using anti-FLAG antibody. (C) The time-dependent change in uptake of 100 μM D-[³H]serine was measured for ASCT2-endogenous hSMCT1- and 2-stable cell lines. (D) Measurement was performed in hSMCT1- and SMCT2-stable cell lines using ASCT2 knockdown. (E) A graph of the observation values for Mock cells subtracted from the observation values for hSMCT1- and SMCT2-stable cell lines, based on the graph of (D). *P<0.05, **P<0.01, NS: No significant difference.

FIG. 4 shows that SMCT transporter-mediated D-serine transport was inhibited by NSAIDs. *P<0.05, **P<0.01.

FIG. 5 shows that D-serine transport activity in mouse brush border membrane vesicles (BBMV) was inhibited by the SMCTs inhibitor ibuprofen. **P<0.01.

FIG. 6 shows that D-serine inhibited proliferation of Flp-In TREx293 cells. Flp-In TREx293 cells were treated for 2 days with L- or D-serine, and cell proliferation was measured by XTT assay. The same data are plotted on a linear curve plot (A) and a semi-logarithmic plot (B). *P<0.05

FIG. 7 shows that D-serine transport was driven by Na⁺-dependent transporter in the cell line. The time-dependent change in 10 μM D-[³H]serine uptake activity was measured using (A) HEK293 cells and (B) an Flp-In TREx 293 cell line. **P<0.01.

FIG. 8 shows that D-serine transport was relatively inhibited by ASCT2 inhibitor. **P<0.01, NS: No significant difference.

FIG. 9 shows that ASCT2 transporter was expressed in Flp-In TREx 293 cells and that KD reduced D-serine toxicity. (A) Using Western blotting it was demonstrated that ASCT2 transporter was endogenously expressed in Flp-In TREx 293 cells. (B) Flp-In TREx 293 control cells and ASCT2 knockdown cells were treated for 2 days with L- or D-serine, and cell proliferation was measured by XTT. *P<0.05.

FIG. 10 shows a D-serine toxicity test in a transient expression system for SMCT1 and SMCT2. The D-serine toxicity test was carried out with HEK293 cells transiently transfected with (A) pCMV14-hSLC5A8-3×FLAG (SMCT1) or (B) pCMV14-hSLC5A12-3×FLAG (SMCT2). *P<0.05.

FIG. 11-1 shows construction of an hSMCT-stable cell line. (A) A vector map of pCDNA5-hSLC5A8-3×FLAG for preparation of an hSMCT1-3×FLAG-stable cell line. (B) A vector map of pCDNA5-hSLC5A12-3×FLAG for preparation of an hSMCT2-3×FLAG-stable cell line.

FIG. 11-2 shows construction of an hSMCT-stable cell line. (C) hSMCT1-3×FLAG (arrow head) and hSMCT2-3×FLAG (arrow) were used in Western blotting with anti-FLAG antibody.

FIG. 12 shows that SMCT2 augmented inhibition of proliferation by D-serine. The cell proliferation effect by serine treatment was examined in an FlpIn293TR-Mock (Mock) or FlpIn293TR-hSLC5A12-3×FLAG (SMCT2)-stable cell line.

FIG. 13 shows that ibuprofen lowered D-serine sensitivity in an SMCT2-stable cell line. *P<0.05, NS: No significant difference.

FIG. 14 shows that SMCT1 increased D-serine sensitivity while ibuprofen canceled out the increased D-serine sensitivity. *P<0.05, NS: No significant difference.

FIG. 15 shows a proteome volcano plot of the kidney brush border membrane fraction in a kidney ischemia reperfusion injury (IRI) mouse. Shown is a volcano plot of log 2 (fold change) (8 h/0 h) with respect to statistically significant difference P value (−log 10), from the data for all of the identified proteins (A) or transporters (B). The known D-serine transporter ASCT2 (SLC1A5) exhibiting 0.54 log 2 (fold change) and 0.80−log 10 P value was used as the cutoff value (arrow).

FIG. 16 shows D-serine induced cytotoxicity for HEK293 cells transfected with a D-serine transporter candidate. HEK293 cells were transfected with each transporter candidate shown in the drawing. The cells were treated for 2 days with D-serine at 15 mM (A) or 25 mM (B) concentration. Cytotoxicity was evaluated by XTT assay. The D-serine toxicity effect in each of the transfected cells was normalized against non-D-serine treatment, and compared with the effect in mock cells at the same D-serine concentration. Significant reduction was calculated by t test. *p<0.05; **p<0.01.

FIG. 17 shows results of identifying SNAT2 as a D-serine transporter in ASCT2-knockout HAP1 cells. (A) D-serine transport was measured in wild type HAP1 cells and ASCT2-knockout HAP1 cells. D-Serine transport was confirmed to be reduced by about 30% in the ASCT2-knockout cells. (B) D-serine transport was measured in wild type HAP1 cells and ASCT2-knockout HAP1 cells, in the presence or in the absence of the SNAT1 and SNAT2 inhibitor MeAIB.

FIG. 18 shows D-serine transport in ASCT2-knockout HAP1 cells stably expressing a cDNA candidate. A cell line stably expressing the indicated cDNA clone was created using ASCT2-knockout HAP1 cells. An ASCT2-stable cell line was used as a positive control. Uptake of [³H]D-serine was measured for 10 minutes in the presence of MeAIB, and the SNAT1 and SNAT2 activity was subtracted from the background. The uptake value was normalized using a mock. The results indicated that SMCT1 had a high level of D-serine uptake. *p<0.05; **p<0.01.

FIG. 19 shows inhibition of [³H]D-serine transport by GABA. (A) Results of measuring [³H]D-serine transport in HEK293 cells. ASCT2 substrate and GABA were shown to inhibit [³H]D-serine uptake. (B) [³H]D-serine transport was measured in HEK293 cells transfected with ASCT2. L-Serine and GABA were added and their inhibiting effects were examined. Both GABA and L-serine notably inhibited [³H]D-serine transport.

### DESCRIPTION OF EMBODIMENTS

The following description will be given on various embodiments for carrying out the present invention, although the technical scope of the present invention should in no way be limited to these embodiments.

The ordinals such as “first,” “second,” . . . , etc., are herein used only for the purpose of distinguishing one element from the others, and therefore have no further implication. Therefore, the term “first” element, for example, is interchangeable with the “second” element and vice versa, and this does not depart from the scope of the invention.

An aspect of the present invention provides a D-serine transport modulator that modulates intracellular and extracellular D-serine transport by a D-serine transport protein.

“D-serine” herein refers to an optical isomer of L-serine, one of the protein-constituting amino acids. The term “D-serine transport protein(s)” (and its shortened form “D-serine transporter(s)”) herein collectively refers to protein(s) that penetrates a biological membrane and transport D-serine through the membrane.

The phrase “intracellular and extracellular transport of D-serine” herein collectively refers to various forms of D-serine transport by means of D-serine transport proteins, including those from inside to outside a cell, from outside to inside a cell, and between multiple cells.

According to one embodiment, the D-serine transport modulator of the present invention may modulate intracellular and extracellular D-serine transport by one or more D-serine transport proteins selected from the group of D-serine transport proteins consisting of SMCT family, GLUT5, CAT1, THTR2 and SNAT2 (hereinafter also referred to as “the first group of D-serine transport proteins”). The term “SMCT family” herein refers to the SMCT protein family including SMCT1 and SMCT2.

SMCT1 refers to the sodium-coupled monocarboxylate transporter 1 protein encoded by the human SLC5A8 gene. The mRNA and amino acid sequences of human SMCT1 are provided in, e.g., the GenBank database and the GenPept database under the accession numbers NM_145913 (SEQ ID NO:1) and NP_666018 (SEQ ID NO:2), respectively, and are thereby available for the present invention.

SMCT2 refers to the sodium-coupled monocarboxylate transporter 2 protein encoded by the human SLC5A12 gene. The mRNA and amino acid sequences of human SMCT2 are provided in, e.g., the GenBank database and the GenPept database under the accession numbers NM_178498 (SEQ ID NO:3) and NP_848593 (SEQ ID NO:4), respectively, and are thereby available for the present invention.

GLUT5 refers to the glucose transporter 5 protein encoded by the human SLC2A5 gene. The mRNA and amino acid sequences of human GLUT5 are provided in, e.g., the GenBank database and the GenPept database under the accession numbers NM_003039 (SEQ ID NO:5) and NP_003030 (SEQ ID NO:6), respectively, and are thereby available for the present invention.

CAT1 refers to the cationic amino acid transporter 1 protein encoded by the SLC7A1 gene. The mRNA and amino acid sequences of human CAT1 are provided in, e.g., the GenBank database and the GenPept database under the accession numbers NM_003045 (SEQ ID NO:7) and NP_003036 (SEQ ID NO:8), respectively, and are thereby available for the present invention.

THTR2 refers to the thiamine transporter 2 protein encoded by the SLC19A3 gene. The mRNA and amino acid sequences of human THTR2 are provided in, e.g., the GenBank database and the GenPept database under the accession numbers NM_025243 (SEQ ID NO:9) and NP_079519 (SEQ ID NO:10), respectively, and are thereby available for the present invention.

SNAT2 refers to the sodium-coupled neutral amino acid transporter 2 protein encoded by the SLC38A2 gene. The mRNA and amino acid sequences of human SNAT2 are provided in, e.g., the GenBank database and the GenPept database under the accession numbers NM_018976 (SEQ ID NO:11) and NP_061849 (SEQ ID NO:12), respectively, and are thereby available for the present invention.

According to another embodiment, the D-serine transport modulator of the present invention may also modulate intracellular and extracellular D-serine transport by, in addition to SMCT family, GLUT5, CAT1, THTR2 and/or SNAT2, one or more other D-serine transport proteins selected from the group of D-serine transport proteins consisting of ASCT family, Asc1, PAT1 and ATB^{0,+} (hereinafter also referred to as “the second group of D-serine transport proteins”). The term “ASCT family” herein refers to the ASCT protein family including ASCT1 and ASCT2.

ASCT1 refers to the sodium-dependent alanine/serine/cysteine/threonine transporter 1 protein encoded by the SLC1A4 gene. The mRNA and amino acid sequences of human ASCT1 are provided in, e.g., the GenBank database and the GenPept database under the accession numbers NM_003038 (SEQ ID NO:13) and NP_003029 (SEQ ID NO:14), respectively, and are thereby available for the present invention.

ASCT2 refers to the sodium-dependent alanine/serine/cysteine/threonine transporter 2 protein encoded by the SLC1A5 gene. The mRNA and amino acid sequences of human ASCT2 are provided in, e.g., the GenBank database and the GenPept database under the accession numbers NM_005628 (SEQ ID NO:15) and NP_005619 (SEQ ID NO:16), respectively, and are thereby available for the present invention.

Asc1 refers to the sodium-independent alanine/serine/cysteine transporter 1 protein encoded by the SLC7A10 gene. The mRNA and amino acid sequences of human Asc1 are provided in, e.g., the GenBank database and the GenPept database under the accession numbers NM_019849 (SEQ ID NO:17) and NP_062823 (SEQ ID NO:18), respectively, and are thereby available for the present invention.

PAT1 refers to the proton-coupled amino acid transporter 1 protein encoded by the SLC36A1 gene. The mRNA and amino acid sequences of human PAT1 are provided in, e.g., the GenBank database and the GenPept database under the accession numbers NM_078483 (SEQ ID NO:19) and NP_510968 (SEQ ID NO:20), respectively, and are thereby available for the present invention.

ATB^{0,+} refers to the sodium- and chloride-dependent neutral and basic amino acid transporter B(^{0,+}) protein encoded by the SLC6A14 gene. The mRNA and amino acid sequences of human ATB^{0,+} are provided in, e.g., the GenBank database and the GenPept database under the accession numbers NM_007231 (SEQ ID NO:21) NP_009162 (SEQ ID NO:22), respectively, and are thereby available for the present invention.

According to one embodiment, the present invention provides a D-serine transport modulator that modulates a D-serine level in a cell, in a tissue, in an organ, or in a body fluid. The expression such as “modulating a D-serine level in a cell” herein refers to modulating a D-serine level in a cell to within a certain range by increasing or decreasing the D-serine level by applying a D-serine transport modulator. The expression such as “modulating a D-serine level in a tissue” herein refers to modulating a D-serine level in a tissue (e.g., kidney tubular or glomeruli) to within a certain range by increasing or decreasing the D-serine level by applying a D-serine transport modulator. The expression such as “modulating a D-serine level in an organ” herein refers to modulating a D-serine level in an organ (e.g., kidney or heart) to within a certain range by increasing or decreasing the D-serine level by applying a D-serine transport modulator. The expression such as “modulating a D-serine level in a body fluid” herein refers to modulating a D-serine level in a body fluid (e.g., blood or urine) to within a certain range by increasing or decreasing the D-serine level by applying a D-serine transport modulator.

According to one embodiment, the present invention provides a D-serine transport modulator which modulates a D-serine level in blood and/or in urine, preferably an excretion ratio of D-serine, which is calculated from a D-serine level in blood and urine. The expression such as “modulating a D-serine level in blood” herein refers to modulating a D-serine level in blood to within a certain range by increasing or decreasing the blood D-serine level by applying a D-serine transport modulator. For example, the D-serine level in blood may be modulated to within the range of 0.5 to 3.0 nmol/mL, preferably 0.7 to 2.5 nmol/mL, more preferably 1.0 to 2.0 nmol/mL. The expression such as “modulating a D-serine level in urine” herein refers to modulating a D-serine level in urine (or an excretion ratio of D-serine) by increasing or decreasing the urine D-serine level by applying a D-serine transport modulator. For example, an excretion ratio of D-serine may be modulated to within the range of 20 to 80%, preferably 30 to 70%, more preferably 40 to 60%.

The term “excretion ratio of D-serine” is herein used to indicate how much of the amount of D-serine filtered by the glomerulus is excreted in the urine through the tubular regulatory functions of reabsorption and secretion. This parameter may be expressed as a percentage, as a ratio, or in other arbitrary units. It may also be expressed as a fractional excretion (FE), as it may be calculated by eliminating the effects of water reabsorption and concentration through correction with a correction factor. Since urine may not have a constant rate of concentration, a “correction factor” for urine concentration may be used to correct the excretion rate of D-serine in the subject. For example, according to one embodiment of the present invention, the excretion rate of D-serine may be corrected with a correction factor derived from blood and/or urine. The excretion rate of D-serine is most simply expressed as the ratio of the D-serine level in urine divided by the glomerular filtration volume of D-serine. This calculation may be based on glomerular filtration volume obtained from inulin clearance or other sources, actual measured urine volume, or the D-serine level in blood. To calculate the excretion rate of D-serine, the amount of an L-amino acid in urine (preferably L-serine) can be used as a urine volume correction factor. A creatinine clearance, calculated by the amount of creatinine in urine or blood, can be used as the correction factor. For example, the excretion rate of D-serine can be expressed by the following formula. This parameter can be expressed as a percentage (%) value by multiplying it by 100.

$$[ Formula 1 ]$$

$$Excretion ratio of D - Ser ( Fe\_D - Ser ) = \frac{{U}_{D - Ser} / {P}_{D - Ser}}{{U}_{Cre} / {P}_{Cre}}$$

[In the formula, U_{D-Ser} represents a D-serine level in urine, P_{D-Ser} represents a D-serine level in blood, U_{cre} represents a creatinine level in urine, and P_{cre} represents a creatinine level in blood.]

D-serine and L-serine levels can be determined by any method, such as chiral column chromatography, enzymatic methods, and even immunological methods using monoclonal antibodies that identify the optical isomers of the amino acids. The measurement of the D-serine and L-serine levels in a sample herein may be performed using any method known to those skilled in the art. Examples include methods such as chromatography and enzyme method (Y. Nagata et al., Clinical Science, 73 (1987), 105. Analytical Biochemistry, 150 (1985), 238, A. D'Aniello et al., Comparative Biochemistry and Physiology Part B, 66 (1980), 319. Journal of Neurochemistry, 29 (1977), 1053., A. Berneman et al., Journal of Microbial & Biochemical Technology, 2 (2010), 139., W. G. Gutheil et al., Analytical Biochemistry, 287 (2000), 196., G. Molla et al., Methods in Molecular Biology, 794 (2012), 273., T. Ito et al., Analytical Biochemistry, 371 (2007), 167., etc.), antibody method (T. Ohgusu et al., Analytical Biochemistry, 357 (2006), 15., etc.), gas chromatography (GC) (H. Hasegawa et al., Journal of Mass Spectrometry, 46 (2011), 502., M. C. Waldhier et al., Analytical and Bioanalytical Chemistry, 394 (2009), 695., A. Hashimoto, T. Nishikawa et al., FEBS Letters, 296 (1992), 33., H. Bruckner and A. Schieber, Biomedical Chromatography, 15 (2001), 166., M. Junge et al., Chirality, 19 (2007), 228., M. C. Waldhier et al., Journal of Chromatography A, 1218 (2011), 4537., etc.), capillary electrophoresis method (CE)(H. Miao et al., Analytical Chemistry, 77 (2005), 7190., D. L. Kirschner et al., Analytical Chemistry, 79 (2007), 736., F. Kitagawa, K. Otsuka, Journal of Chromatography B, 879 (2011), 3078., G. Thorsen and J. Bergquist, Journal of Chromatography B, 745 (2000), 389., etc.), high-performance liquid chromatography (HPLC)(N. Nimura and T. Kinoshita, Journal of Chromatography, 352 (1986), 169., A. Hashimoto et al., Journal of Chromatography, 582 (1992), 41., H. Bruckner et al., Journal of Chromatography A, 666 (1994), 259., N. Nimura et al., Analytical Biochemistry, 315(2003), 262., C. Muller et al., Journal of Chromatography A, 1324 (2014), 109., S. Einarsson et al., Analytical Chemistry, 59 (1987), 1191., E. Okuma and H. Abe, Journal of Chromatography B, 660 (1994), 243., Y. Gogami et al., Journal of Chromatography B, 879 (2011), 3259., Y. Nagata et al., Journal of Chromatography, 575 (1992), 147., S. A. Fuchs et al., Clinical Chemistry, 54 (2008), 1443., D. Gordes et al., Amino Acids, 40 (2011), 553., D. Jin et al., Analytical Biochemistry, 269 (1999), 124., J. Z. Min et al., Journal of Chromatography B, 879 (2011), 3220., T. Sakamoto et al., Analytical and Bioanalytical Chemistry, 408 (2016), 517., W. F. Visser et al., Journal of Chromatography A, 1218 (2011), 7130., Y. Xing et al., Analytical and Bioanalytical Chemistry, 408 (2016), 141., K. Imai et al., Biomedical Chromatography, 9 (1995), 106., T. Fukushima et al., Biomedical Chromatography, 9 (1995), 10., R. J. Reischl et al., Journal of Chromatography A, 1218 (2011), 8379., R. J. Reischl and W. Lindner, Journal of Chromatography A, 1269 (2012), 262., S. Karakawa et al., Journal of Pharmaceutical and Biomedical Analysis, 115 (2015), 123., etc.).

The separation analysis system for optical isomers in the present invention may combine two or more separation analysis methods. More specifically, the amounts of D-/L-amino acids in a sample can be measured by using a method for analyzing optical isomers comprising the following steps (JP4291628B): passing a sample containing components having optical isomers through a first column packing material as a stationary phase together with a first liquid as a mobile phase to separate said component of said sample; retaining each of the components from the sample separately in a multi-loop unit; feeding each of the components from the sample separately retained in the multi-loop unit, together with a second liquid as a mobile phase, through a channel to a second column packing material having an optically active center as a stationary phase, to separate the optical isomers of each component from the sample; and detecting the optical isomers of each component from the sample. For HPLC analysis, D- and L-amino acids may be derivatized beforehand with fluorescent reagents such as o-phthalaldehyde (OPA) and 4-fluoro-7-nitro-2,1,3-benzoxadiazole (NBD-F) or diastereomerized with N-tert-butyloxycarbonyl-L-cysteine (Boc-L-Cys) and others (Kenji Hamase and Kiyoshi Zaitsu, Analytical Chemistry, Vol. 53, 677-690 (2004)). Alternatively, D-amino acids can be measured by an immunological method using monoclonal antibodies that identify optical isomers of amino acids, e.g., monoclonal antibodies that bind specifically to D-serine, L-serine, etc. On the other hand, when the total amount of D- and L-isomers of an amino acid can be used as an indicator, it is not necessary to analyze D- and L-isomers separately, but the amino acid can be analyzed without distinguishing between D- and L-isomers. Also in this case, separation and quantification can be performed via enzyme methods, antibody methods, GC, CE, HPLC, etc.

According to one embodiment, the D-serine transport modulator of the present invention may act on the D-serine transport protein expressed in kidney cells. This makes it possible to modulate the D-serine level(s) in the blood and/or urine, and preferably the rate of D-serine excretion.

According to one embodiment, the D-serine transport modulator of the present invention may inhibit D-serine transport into a cell by acting on a D-serine transport protein. For example, the D-serine transport modulator of the present invention may be a substrate or inhibitor (e.g., selective inhibitor or non-selective inhibitor) of the D-serine transport protein, or a substance that directly inhibits the expression of the D-serine transport protein, or a substance that directly or indirectly suppress a gene associated with the expression of the D-serine transport protein. The term “selective inhibitor of a D-serine transport protein” herein refers to an inhibitor that exhibits its inhibitory activity by acting on the D-serine transport protein in a selective manner, such as an inhibitor having a Ki value to the D-serine transport protein which is ⅕ or less, preferably 1/10 or less, more preferably 1/25 or less, still more preferably 1/100 or less, of its Ki values to other proteins. The Ki value of the selective inhibitor of a D-serine transport protein can be measured using various methods well-known to the art. The selective inhibitor of a D-serine transport protein may be selected from low-molecular compounds, aptamers, antibodies, antibody fragments, and combinations thereof. On the other hand, the term “non-selective inhibitor of a D-serine transport protein” herein refers to an inhibitor that exhibits its inhibitory activity by acting on the D-serine transport protein in a non-selective manner.

The term “low-molecular compounds” herein refers to molecules that are comparable in size to organic molecules commonly used in pharmaceuticals, e.g., compounds with molecular weights in the range of about 5000 Da or less, preferably about 2000 Da or less, more preferably about 1000 Da or less. According to one embodiment, the D-serine transport modulator of the present invention may be one or more low-molecular compounds selected from the group of substrates and inhibitors of D-serine transport proteins selected from the first group of D-serine transport proteins consisting of SMCT family, GLUT5, CAT1, THTR2 and SNAT2 (hereinafter also referred to as “first group of substrates and inhibitors”). According to one embodiment, the D-serine transport modulator of the present invention may be selected from, although not limited to, the group of low-molecular compounds that can act as substrates or inhibitors of SMCT1, such as ibuprofen, fenoprofen, ketoprofen, probenecid, acetyl salicylic acid, naproxen, pyroglutamic acid, phenoxyacetic acid, acetic acid, propionic acid, butyric acid, L-lactic acid, D-lactic acid, pyruvic acid, nicotinic acid, acetoacetic acid, β-D-hydroxybutyric acid, β-L-hydroxybutyric acid, γ-hydroxybutyric acid, α-ketoisocaproic acid, benzoic acid, salicylic acid, 5-amino salicylic acid, 2,4-dichlorophenoxyacetic acid (2,4-D), 4-chlorophenoxyacetic acid (4-CPA), 2-chlorophenoxyacetic acid (2-CPA), 2,3-dichlorophenoxyacetic acid, 3,4-dichlorophenoxyacetic acid, 2,4,5-trichlorophenoxyacetic acid and its derivatives, and pharmaceutically acceptable salts thereof.

The term “derivative” is herein intended to encompass a molecule such as a compound or a protein partly modified with various substituents or sugar chains. The term “pharmaceutically acceptable salts thereof” herein refers to any forms of non-toxic salts of a substance that can be used as the D-serine transport modulator. Such salts can be obtained via reaction with, e.g., inorganic salts such as hydrochloric acid, sulfuric acid, phosphoric acid, hydrobromic acid; organic acids such as oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, and benzilsulfonic acid; inorganic bases such as sodium hydroxide, potassium hydroxide, calcium hydroxide, magnesium hydroxide, and ammonium hydroxide; organic bases such as methylamine, diethylamine, triethylamine, triethanolamine, ethylenediamine, tris(hydroxymethyl)methylamine, guanidine, choline, and syncholine; or amino acids such as lysine, arginine, and alanine. The term “pharmaceutically acceptable salts thereof” herein also encompasses hydrous products and solvates (e.g., hydrates) of substances used as D-serine transport regulators.

According to one embodiment, the D-serine transport modulator of the present invention may be a low-molecular compound that functions as a substrate or inhibitor of SMCT2. Examples thereof may be selected from, although not limited to, the group consisting of ibuprofen, fenoprofen, ketoprofen, probenecid, acetyl salicylic acid, naproxen, pyroglutamic acid, phenoxyacetic acid and its derivatives, and pharmaceutically acceptable salts thereof.

According to one embodiment, the D-serine transport modulator of the present invention may be a low-molecular compound that functions as a substrate or inhibitor of GLUT5. Examples thereof may be selected from, although not limited to, the group consisting of N-(4-methane sulphonyl-2-nitrophenyl)-2H-1,3-benzodioxol-5-amine (MSNBA), fructose and its derivatives, and pharmaceutically acceptable salts thereof.

According to one embodiment, the D-serine transport modulator of the present invention may be a low-molecular compound that functions as a substrate or inhibitor of CAT1. Examples thereof may be selected from, although not limited to, the group consisting of N-ethylmaleimide (NEM), N-amino-L-arginine, N-amino-L-homoarginine, L-arginine, L-histidine, L-lysine, L-ornithine and its derivatives, and pharmaceutically acceptable salts thereof.

According to one embodiment, the D-serine transport modulator of the present invention may be a low-molecular compound that functions as a substrate or inhibitor of THTR2. Examples thereof may be selected from, although not limited to, the group consisting of metformin, chloroquine, 2,4-diamino pyrimidine, drugs having 2,4-diamino pyrimidine group (e.g., Fedratinib, AZD1480, Cerdulatinib), thiamine and its derivatives, and pharmaceutically acceptable salts thereof.

According to one embodiment, the D-serine transport modulator of the present invention may be a low-molecular compound that functions as a substrate or inhibitor of SNAT2. Examples thereof may be selected from, although not limited to, the group consisting of methyl-amino-isobutyric acid (MeAIB), γ-glutamyl-p-nitroanilide (GPNA), 2-amino-4-bis(aryloxybenzil)amino butyric acid (AABA), L-alanine, L-methionine, L-proline, L-serine, L-asparagine, L-glutamine, L-histidine, glycine and its derivatives, and pharmaceutically acceptable salts thereof.

According to another embodiment, the D-serine transport modulator of the present invention may contain, in addition to one or more low-molecular compounds selected from the first group of substrates and inhibitors, one or more other low-molecular compounds selected from a group of substrates and inhibitors of a D-serine transport protein selected from the second group of D-serine transport proteins consisting of ASCT family, Asc1, PAT1 and ATB^{0,+} (hereinafter also referred to as the “second group of substrates and inhibitors”). For example, it may contain a substance selected from the group consisting of substrates or inhibitors of ASCT family (e.g., ASCT1 and ASCT2), including phenylglycine analogs (e.g., L-4-fluorophenylglycine, L-4-chlorophenylglycine, etc.), benzilserine, benzilcysteine, S-benzil-L-cystine, L-γ-glutamyl-p-nitroanilide, L-serine, L-threonine, L-methionine, L-alanine, L-cysteine, L-glutamine, D-alanine and its derivatives, and pharmaceutically acceptable salts thereof; and/or a substance selected from the group consisting of substrates or inhibitors of Asc1, including phenylglycine analogs (e.g., L-4-bromophenylglycine, L-4-hydroxyphenylglycine, etc.), alanine analogs (e.g., 2-aminoisobutyl acid (AIB), etc.), L-serine, L-alanine, L-cysteine, glycine, L-threonine and its derivatives, and pharmaceutically acceptable salts thereof; and/or a substance selected from the group consisting of substrates or inhibitors of PAT1, including taurine, GABA, tryptophan, tryptamine derivative, 5-hydroxy-L-tryptophan, serotonin, indole-3-propionic acid, and its derivatives, and pharmaceutically acceptable salts thereof; and/or a substance selected from the group consisting of substrates or inhibitors of ATB^{0,+}, including α-methyl-DL-tryptophan, and its derivatives, and pharmaceutically acceptable salts thereof.

The term “aptamer” herein refers to synthetic DNA or RNA molecules and peptide molecules that have the ability to bind specifically to a target substance and can be chemically synthesized in vitro in a short time. Aptamers used in the present invention may, e.g., bind to the D-serine transport protein and inhibit the activity of the D-serine transport protein. Aptamers used in the present invention can be obtained, for example, by using the SELEX method to select for binding to various molecular targets, such as small molecules, proteins, and nucleic acids, in vitro and repeatedly (see, e.g., Tuerk C., Gold L., Science, 1990, 249 (4968):505-510; Ellington A D, Szostak J W., Nature, 1990, 346 (6287):818-822; U.S. Pat. Nos. 6,867,289; 5,567,588; and 6,699,843).

The term “antibody fragment” herein refers to a portion of a full-length antibody that retains its activity to bind to an antigen, and generally includes its antigen-binding domain or variable domain. Examples of antibody fragments include F(ab′)2, Fab′, Fab, or Fv antibody fragments (such as scFv antibody fragments). Other examples of antibody fragments are fragments that can be obtained by treating antibodies with protease enzymes and possibly reducing them. The antibodies or antibody fragments used in the present invention may be human-derived, mouse-derived, rat-derived, rabbit-derived, llama or other camelid-derived, or goat-derived antibodies, and may be polyclonal or monoclonal antibodies, complete or shortened (e.g., F(ab′)2, Fab′, Fab or Fv fragment), chimeric, humanized or fully human antibodies thereof.

According to one embodiment, the D-serine transport modulator of the present invention may directly or indirectly inhibit the expression of D-serine transport proteins and may be selected from, e.g., low-molecular compounds, aptamers, antibodies, antibody fragments, and antisense RNA or DNA molecules, RNAi inducible nucleic acids, microRNAs (miRNA), ribozymes, genome-editing nucleic acids, and expression vectors thereof.

The term “antisense RNA or DNA molecule” herein refers to a molecule that has a complementary base sequence to RNA with a certain function (sense RNA), such as messenger RNA (mRNA), and has the function of inhibiting the synthesis of the protein that the sense RNA should carry out, by forming a double-stranded RNA with the sense RNA. In the present invention, antisense oligonucleotides containing antisense RNA or DNA molecules inhibit the translation into the D-serine transport protein by binding to its mRNA. This reduces the expression of the D-serine transport protein and inhibits the activity of the D-serine transport protein. Methods for synthesizing antisense RNA or DNA molecules are well known in the art and can be used in the present invention.

The term “RNAi inducible nucleic acid” herein refers to a polynucleotide that can induce RNA interference (RNAi) when introduced into cells, and is usually between 19 and 30 nucleotides, preferably 19 to 25 nucleotides, more preferably 19 to 23 nucleotides-containing DNA, or RNA and DNA chimeric molecules, which may optionally be modified. RNAi may occur on mRNA or on RNA immediately after transcription before processing, i.e., RNA of nucleotide sequences including exons, introns, 3′ untranslated regions, and 5′ untranslated regions. RNAi methods that can be used in this invention include, for example: (1) a short double-stranded RNA (siRNA) may be introduced directly into the cells; (2) small molecule hairpin RNA (shRNA) may be incorporated into various expression vectors and introduced into the cells; or (3) a vector having two promoters aligned in the opposite directions may be modified by inserting a short double-stranded DNA corresponding to the siRNA between the promoters to create a vector for siRNA expression, and the resulting vector may be introduced into the cells. RNAi inducible nucleic acids may include siRNAs, shRNAs or miRNAs that allow cleavage of the RNA of D-serine transport protein or suppression of its function. These RNAi nucleic acids may be introduced directly using liposomes or other means or indirectly by expression vectors that induce these RNAi nucleic acids.

According to one embodiment, an RNAi inducible nucleic acids against the D-serine transport protein used in the present invention may be any nucleic acid that has a biological effect of inhibiting or significantly suppressing the expression of the D-serine transport protein, and can be synthesized by a person skilled in the art with reference to the nucleotide sequence of the D-serine transport protein. For example, DNA (/RNA) can be chemically synthesized using an automated DNA (/RNA) synthesizer utilizing DNA synthesis techniques such as solid-phase phosphoramidite methods. RNA) can be chemically synthesized using an automated synthesizer. Alternatively, siRNA-related contract synthesis companies (e.g. Life Technologies, Inc. Ltd.) to synthesize the siRNA. In one embodiment, an siRNAs used in the present invention may be derived from a precursor of a short-hairpin-type double-stranded RNA (shRNA) to an intracellular RNase via processing by Dicer, an intracellular RNase.

The term “micro RNA (miRNA)” herein refers to a single-stranded RNA molecule 21-25 bases long that is involved in the post-transcriptional regulation of gene expression in eukaryotes. In general, a miRNA recognizes the 3′ UTR of an mRNA to suppress the translation of the target mRNA, and thereby suppress the production of the protein. Thus, a miRNA that can directly and/or indirectly reduce the expression of the D-serine transport protein is also within the scope of the invention.

The term “ribozyme” herein collectively refers to enzymatic RNA molecules capable of catalyzing the specific cleavage of RNA. Some ribozymes such as Group I intronic type and M1 RNA are as large as 400 nucleotides or more, while other ribozymes such as hammerhead and hairpin types have active domains of as small as about 40 nucleotides (see, e.g., Makoto Koizumi and Eiko Otsuka, Proteins, Nucleic Acids, and Enzymes, 1990, 35, 2191).

The self-cleavage domain of a hammerhead ribozyme, for example, can cause cleavage at C15 on the 3′ side of the sequence G13U14C15, and its activity relies on base pairing between U14 and A9, although the cleavage may also occur at A15 or U15 instead of C15 (e.g., Koizumi, M. E. et al. FEBS Lett, 1988, 228, 228). By designing a ribozyme whose substrate binding site is complementary to the RNA sequence near the target site, it is possible to obtain a restriction enzymatic RNA-cleaving ribozyme that recognizes the sequence UC, UU or UA in the target RNA, which can be produced by a person skilled in the art with reference to the following literature: Koizumi, M. et al., FEBS Lett, 1988, 239, 285; Makoto Koizumi and Eiko Otsuka, Tarnpak enzymes, 1990, 35, 2191; Koizumi, M. et al., Nucl. Acids Res., 1989, 17, 7059.

A hairpin-type ribozyme can also be used in the present invention. This ribozyme is found, for example, in the minus strand of the satellite RNA of tobacco ringspot virus (Buzayan, J M. Nature, 198. 6, 323, 349). It has been shown that a target-specific RNA-cleaving ribozyme can also be produced from hairpin ribozymes (e.g. Kikuchi, Y. & Sasaki, N., Nucl. Acids. Res. 1991, 19, 6751; Hiroshi Kikuchi, Chemistry and Biology, 1992, 30, 112). Such a ribozymes can be used to specifically cleave the transcript of the gene encoding the D-serine transport protein to thereby inhibit the expression of the D-serine transport protein.

The term “genome-editing nucleic acids” herein refer to nucleic acids used to edit a desired gene in a system utilizing nucleases used for gene targeting. The nucleases used for gene targeting in this context not only include known nucleases but also encompass novel nucleases that will be used for gene targeting in the future. Examples of known nucleases include CRISPR/Cas9 (Ran, F. A., et al., Cell, 2013, 154, 1380-1389), TALEN (Mahfouz, M., et al., PNAS, 2011, 108, 2623-2628), and ZFN (Urnov, F., et al., Nature, 2005, 435, 646-651).

The CRISPR/Cas9 system, which can be used in an embodiment of the present invention, will be described below.

The CRISPR/Cas 9 system allows the introduction of a double-strand break at any site in DNA. In order to use the CRISPR/Cas 9 system, at least a protospacer adjacent motif (PAM sequence), a guide RNA (gRNA), and a Cas protein (Cas, Cas 9) are required.

The gRNA is designed to form a complementary sequence to the target site adjacent to the PAM sequence (5′-NGG), and is introduced with the Cas protein into the desired cells. The introduced gRNA forms a complex with the Cas protein. The gRNA binds to the target sequence on the genome, and the Cas protein cleaves the duplex of the target genomic DNA by its nuclease activity.

Homology Directed Repair (HDR) or non-homologous end joining (NHEJ) repair then occurs in a cell that has undergone double-strand breaks by the nuclease. If an appropriate DNA fragment (e.g., a template for HDR repair) is present in the cell, homologous recombination can occur, whereby an alteration such as a deletion, insertion, or disruption can be made in any genome. If there is no template for HDR repair, deletion or addition of a few bases may occur during the NHEJ process. This can result in a frameshift in the protein coding region, disrupting the protein reading frame or introducing an immature termination codon, resulting in the knockout of the desired protein.

According to one embodiment of the present invention, the genome-editing nucleic acid may be a gRNA targeting a gene encoding a D-serine transport protein or a vector expressing it. According to another embodiment, genome-editing nucleic acid may further include a nucleic acid expressing a nuclease used for gene targeting. The gRNA and the nuclease (preferably the Cas protein) used for gene targeting may be encoded in the same vector or in separate vectors. According to another embodiment, the genome-editing nucleic acid may further contain a template nucleic acid for HDR repair.

According to one embodiment, the present invention provides a pharmaceutical composition for the treatment or prevention of diseases associated with an increase in the D-serine level in a cell, in a tissue, in an organ, or in a body fluid, containing the D-serine transport modulator as an active ingredient.

The D-serine transport modulator according to the present invention, or the pharmaceutical composition containing the D-serine transport modulator, can be administered by any route of administration, provided that the concentration at the site of action can be properly adjusted. Examples of administration routes include topical (e.g., dermal, inhalation, intravenous, ophthalmic, ear, nasal, intravaginal, etc.), enteral (e.g., oral, tube, intravenous, etc.), and parenteral (e.g., intravenous, transarterial, transdermal, intramuscular, etc.) administrations.

The term “diseases associated with an increase in the D-serine level in a cell, in a tissue, in an organ, or in a body fluid” herein refers to a disease correlated with elevated levels of D-serine in a cell, in a tissue, in an organ, or in a body fluid, such as a kidney disease. According to one embodiment, the “kidney disease” to which this invention may be applied is a condition involving glomeruli and/or kidney tubular disorders, examples of which include acute kidney disorder, chronic kidney disease, myeloma kidney, diabetic nephropathy, IgA nephropathy, interstitial nephritis, or polycystic kidney disease, or kidney diseases involved in systemic lupus erythematosus, primary aldosteronism, benign prostatic hyperplasia, Fabry's disease, or microvascular nephrotic syndrome.

According to one embodiment, the present invention provides a method for treating or preventing diseases associated with an increase in the D-serine level in a cell, in a tissue, in an organ, or in a body fluid, comprising administering the D-serine transport modulator to a subject in need thereof. The term “treating” refers to the alleviation or elimination of an afflicted disease or disorder and/or associated symptoms, which can be assessed by, e.g., confirming the recovery and/or maintenance of glomerular filtration rate. The term “preventing” herein refers to the prevention of the onset of a disease or disorder.

According to one embodiment, the present invention provides a D-serine transport modulator that enhances D-serine transport by a D-serine transport protein into a cell by acting on the D-serine transport protein.

According to one embodiment of the present invention, the D-serine transport modulator that enhances D-serine transport into a cell by acting on a D-serine transport protein may be, for example, a vector expressing the D-serine transport protein, a derivative thereof, or a portion thereof, or a substance that directly or indirectly upregulates the expression of the D-serine transport protein. It may be selected, for example, from low-molecular compounds, aptamers, antibodies, antibody fragments, and antisense RNA or DNA molecules, RNAi inducible nucleic acids, micro RNA (miRNA), ribozymes, genome-editing nucleic acids, and their expression vectors.

The term “vector” herein refers to a nucleic acid molecule (carrier) capable of transporting a nucleic acid molecule inserted therein into a target such as a cell. The type and structure of such vectors are not limited as long as the inserted nucleic acid molecule can be replicated and expressed in an appropriate host cell. The vector may be selected from the group consisting of plasmid vectors, cosmid vectors, fosmid vectors, artificial chromosome vectors, and virus vectors. According to one embodiment of the present invention, any known methods can be used for introducing the vector into a cell.

The “vector expressing the D-serine transport protein, a derivative thereof, or a part thereof” herein refers to a vector which carries a nucleic acid encoding the D-serine transport protein, a derivative thereof, or a part thereof inserted therein, and which is capable of expressing the D-serine transport protein, its derivative, or its portion when introduced into suitable host cells. The “vector expressing the D-serine transport protein, a derivative thereof, or a part thereof” that can be used in the present invention may include any vector capable of expressing the D-serine transport protein having an amino acid sequence with a homology (preferably identity) of at least 85% or more, preferably 90% or more, more preferably 95% or more, still more preferably 97% or more, most preferably 99% or more to the amino acid sequence of the D-serine transport protein mentioned above. The “vector expressing the D-serine transport protein, a derivative thereof, or a part thereof” that can be used in the present invention may also be any vector capable of expressing the D-serine transport protein having an amino acid sequence with a homology (preferably identity) of at least 85% or more, preferably 90% or more, more preferably 95% or more, still more preferably 97% or more, most preferably 99% or more to the amino acid sequence of the D-serine transport protein mentioned above and in which an amino acid sequence in the substrate (e.g., D-serine) binding site is conserved.

The “homology” between two amino acid sequences herein refers to the ratio of identical or similar amino acid residues appearing at each corresponding location when both amino acid sequences are aligned, and the “identity” between two amino acid sequences herein refers to the ratio of identical amino acid residues appearing at each corresponding location when both amino acid sequences are aligned.

The “homology” and the “identity” between two amino acid sequences can be determined, for example, with the Needle program of the EMBOSS package (EMBOSS: The European Molecular Biology Open Software Suite, Rice et al., 2000, Trends Genet. 16: 276-277) (preferably version 5.0 0 or later), using the Needleman-Wunsch algorithm (Needleman and Wunsch, 1970, J. Mol. Biol. 48: 443-453).

Similar amino acids include, for example, amino acids that belong to the same one of the following groups, which are classified based on structures, characteristics, and types of their side chains:
Aromatic amino acids: F, H, W, Y;
Aliphatic amino acids: I, L, V;
Hydrophobic amino acids: A, C, F, H, I, K, L, M, T, V, W, Y;
Charged amino acids: D, E, H, K, R, etc.:
Positively charged amino acids: H, K, R;
Negatively charged amino acids: D, E;
Polar amino acids: C, D, E, H, K, N, Q, R, S, T, W, Y;
Small amino acids: A, C, D, G, N, P, S, T, V, etc.:
Micro amino acids: A, C, G, S;
Amino acids having aliphatic side chains: G, A, V, L, I;
Amino acids having aromatic side chains: F, Y, W;
Amino acids having sulfur-containing side chains: C, M;
Amino acids having aliphatic hydroxyl side chains: S, T;
Amino acids having basic side chains: K, R, H; and
Acidic amino acids and their amide derivatives: D, E, N, Q.

According to one embodiment of the present invention, the D-serine transport modulator that enhances D-serine transport into a cell by acting on a D-serine transport protein may be selected from the group consisting of, for example, diclofenac, curcumin, activin A, and SMCT family-, GLUT5-, CAT1-, THTR2-, SNAT2-, and PDZK1-expression vectors.

The term “PDZK1” herein refers to a scaffold protein called PDZ domain containing 1, and increased expression of this protein is known to increase the expression and activity of SMCT family proteins at the membrane (see, e.g., Liu Y., et al., Drug Metab Pharmacokinet. 2013; 28(2):153-8.; Srivastava S., et al., J Physiol Sci. 2019 March; 69(2):399-408). The mRNA and amino acid sequences of human PDZK1 are provided in, e.g., the GenBank database and the GenPept database, under Accession Numbers NM_002614 (SEQ ID NO:23) and NP_002605 (SEQ ID NO:24), respectively. The “vector expressing PDZK1, a derivative thereof, or a part thereof” that can be used in the present invention is a vector carrying a nucleic acid encoding the PDZK1 protein, a derivative thereof, or a part thereof inserted therein, and is capable of expressing the PDZK1 protein, derivative, or part when introduced into appropriate host cells. The “vector expressing PDZK1, a derivative thereof, or a part thereof” that can be used in the present invention may include any vector capable of expressing a PDZK1 protein having an amino acid sequence with a homology (preferably identity) of at least 85% or more, preferably 90% or more, more preferably 95% or more, still more preferably 97% or more, most preferably 99% or more to the amino acid sequence of the PDZK1 protein mentioned above.

According to one embodiment, the present invention provides a pharmaceutical composition for the treatment or prevention of diseases associated with a decrease in a D-serine level in a cell, in a tissue, in an organ, or in a body fluid, comprising the D-serine transport modulator as an active ingredient.

The term “diseases associated with a decrease in a D-serine level in a cell, in a tissue, in an organ, or in a body fluid” herein refers to diseases correlated with decreased levels of D-serine in a cell, in a tissue, in an organ, or in a body fluid, such as a kidney disease. According to one embodiment, the “kidney disease” to which this invention may be applied is a condition involving glomeruli and/or kidney tubular disorders, examples of which include acute kidney disorder, chronic kidney disease, myeloma kidney, diabetic nephropathy, IgA nephropathy, interstitial nephritis, or polycystic kidney disease, or kidney diseases involved in systemic lupus erythematosus, primary aldosteronism, benign prostatic hyperplasia, Fabry's disease, or microvascular nephrotic syndrome.

According to one embodiment, the present invention provides a method for treating or preventing diseases associated with a decrease in a D-serine level in a cell, in a tissue, in an organ, or in a body fluid, comprising administering the D-serine transport modulator to a subject in need thereof.

According to one embodiment, the present invention provides a method of screening a substance that modulates intracellular and extracellular D-serine transport by a D-serine transport protein, comprising applying a candidate substance and D-serine to a cell expressing a D-serine transport protein to evaluate the degree of intracellular and extracellular D-serine transport based on the expression of cytotoxicity as an indicator.

The term “candidate substances” herein refers to a substance to be screened and may include, but are not limited to, low-molecular compounds, peptides, proteins, tissue extracts or cell culture supernatants from mammals (e.g., mouse, rat, pig, cow, sheep, monkey, human, etc.), compounds or extracts derived from plants (e.g., herbal medicine extracts, compounds derived from crude drugs), and compounds, extracts or culture products from microorganisms.

The term “expression of cytotoxicity” herein refers to the occurrence of an event that causes some injury to living cells (e.g., cell death, modulation or reduction of various cellular functions such as proliferative capacity, metabolic capacity, etc.). According to one embodiment, the cells that can be used in the screening method of the present invention are of animal origin, preferably of mammalian origin (e.g., human, non-human primate, rodent (e.g., mouse, rat, hamster, guinea pig, etc.), rabbit, dog, cow, horse, pig, cat, goat, sheep, etc.), more preferably human cells and non-human cells derived from primates. The cells that can be used in the screening method of the present invention may be, but are not limited to, brain-derived or kidney-derived cells.

According to one embodiment, the cells that can be used in the screening method of the present invention may be kidney- or brain-derived cells in which the D-serine transport protein is expressed. According to another embodiment, the cells that can be used in the screening method of the present invention may express one or more D-serine transport proteins selected from the first group of D-serine transport proteins from the SMCT family, GLUT5, CAT1, THTR2, and SNAT2, and may further express, in addition to one or more proteins selected from the first group of D-serine transport proteins, one or more D-serine transport proteins selected from the second group of D-serine transport proteins consisting of the ASCT family, Asc1, PAT1 and ATB^{0,+}.

According to one embodiment, the cells that can be used in the screening method of the present invention may be cells obtained by externally introducing a vector expressing the D-serine transport protein.

According to one embodiment, the screening method of the present invention may comprise selecting substances which inhibit D-serine transport into a cell by acting on the D-serine transport protein to screen for the effect of treating or preventing a disease (e.g., kidney disease) associated with an increase in the D-serine level in a cell, in a tissue, in an organ, or in a body fluid. According to one embodiment, the screening method of the present invention may comprise selecting substances which enhance D-serine transport into a cell by acting on the D-serine transport protein to screen the substances for the effect of treating or preventing a disease (e.g., kidney disease) associated with a decrease in a D-serine level in a cell, in a tissue, in an organ, or in a body fluid.

According to one embodiment, the present invention provides a method of screening a D-serine transport protein based on D-serine transport into a cell as an indicator.

According to one embodiment of the present invention, the “indicator of the D-serine transport into a cell” may be any indicator showing that D-serine was transported into a cell. The indicator may be, for example, the amount of radioisotope labeled D-serine taken into the cell by adding D-serine labeled with a radioisotope to the cell, which is measured by a scintillation counter or the like. Alternatively, the degree of expression of cytotoxicity produced by the addition of D-serine may be used as an indicator of the transport of D-serine into a cell. Among them, it is preferable to use the degree of expression of cytotoxicity induced by the addition of D-serine as an “indicator of D-serine transport into cells” to simplify the assay system.

According to one embodiment, the cells to be used in the present invention may be cells that express the candidate transporter protein, e.g., cells obtained by introducing a vector expressing the candidate transporter protein. The term “candidate transporter protein” herein refers to a protein which is known as a membrane transporter protein but not known as a D-serine transport protein. The candidate transporter protein that can be applied to the screening method of the present invention can be obtained from their gene sequences available on gene databases and the like. Specifically, a person skilled in the art can produce a vector expressing the candidate transporter protein by consulting such a known gene database or the like.

According to one embodiment, the screening method of the present invention may also involve applying, in addition to D-serine, additional ions selected from the group consisting of sodium ions, potassium ions, protons, and chloride ions.

### EXAMPLES

The present invention will be described in more detail with reference to Examples which by no means limit the scope of the present invention.

### Example 1

### 1. Materials

General chemicals were purchased from FujiFilm Corp. unless otherwise specified. Cell culture media were acquired from FujiFilm Corp., and bacterial culture media were acquired from Nacalai Tesque, Inc. Restriction enzymes were acquired from New England BioLabs, Massachusetts, USA. DNA primer synthesis and DNA sequencing was conducted using a Macrogen System (Korea). Flp-In TREx293 cell lines were acquired from Invitrogen, California, USA, and fetal calf serum, p3×FLAG-CMV-14 expression vectors and HRP-binding anti-FLAG M2 monoclonal antibodies (anti-FLAG-HRP) were purchased from Sigma-Aldrich, Missouri, USA. ASCT2-siRNA was acquired from Thermo Fisher Scientific, Massachusetts, USA. D-[³H]serine (10 Ci/mmol) was purchased from Moravek, California, USA. Anti-SMCT2 (H4) monoclonal antibody was acquired from Santa Cruz Biotechnology, Texas, USA. For the animal experiments, 8-week-old male C57BL/6 J mice (body weight: 21 to 27 g) were purchased from Japan SLC, Inc. and were reared in groups of up to 4 per cage under a 12-hour light/dark cycle with feeding. The animal experiment was conducted according to the guidelines established by the Experimental Animal Use Committee of Nara Medical University.

### 2. Experiment Protocol

2-1. Preparation of Brush Border Membrane Vesicles (BBMVs) from Mouse Kidneys

Total mouse kidneys were extracted by perfusion using PBS buffer at pH 7.4, and then immediately frozen with liquid nitrogen and stored at −80° C. until use. The frozen kidneys were resuspended in buffer containing 20 mM Tris-HCl at pH 7.6, 250 mM mannitol, 1 mM EDTA and Complete EDTA-free protease inhibitor cocktail (Roche, Switzerland), and crushed using a PHYSCOTRON (Microtec Co., Ltd.). The sample was then homogenized using a Potter-Elvehjem homogenizer. After centrifugation at 1,000×g for 5 minutes at 4° C., the supernatant was centrifuged at 3,000×g for 5 minutes at 4° C. After centrifugation, the supernatant was mixed with 1 M MgCl₂ at a final concentration of 11 mM MgCl₂, and incubated on ice for 20 minutes. This was followed by centrifugation at 3,000×g for 15 minutes at 4° C., and then ultracentrifugation of the supernatant containing crude membrane vesicles at 438,000×g for 30 minutes at 4° C. The pellet remaining after ultracentrifugation (BBMVs) was suspended in buffer containing 20 mM Tris-HCl at pH 7.6 and 250 mM mannitol. The BBMVs were quantified using a BCA protein quantitation kit (Thermo Fisher Scientific). The BBMVs were cryopreserved at −80° C. until use.

2-2. Transport Activity Measurement with Mouse BBMVs

Before transport activity measurement, the cryopreserved BBMVs were thawed to allow introduction of potassium into the BBMVs, after which they were ultracentrifuged at 438,000×g for 30 minutes at 4° C., and the pellet was then suspended in buffer containing 10 mM Tris-HCl at pH 7.6, 100 mM KCl and 100 mM mannitol to a 5 mg/mL protein concentration, and the BBMV suspension was incubated on ice overnight. Before measurement of the transport activity, valinomycin was added to the BBMV sample to a final concentration of 5 μM and the mixture was incubated at room temperature for 30 minutes. The transport activity measurement was initiated by diluting the BBMV sample (100 μg) 5-fold in uptake buffer (containing 10 mM Tris-HCl at pH 7.6, 150 mM NaCl, or KCl in place of NaCl, 50 mM mannitol, D-[³H]serine). The reactants were incubated in a 30° C. thermostatic bath for the indicated time, and the reaction was stopped by addition of thoroughly cooled buffer containing 10 mM Tris-HCl at pH 7.6 and 200 mM mannitol. Filtration was carried out through a 0.45 μm nitrocellulose filter (Millipore), and followed by washing once with the same buffer. The used filter was melted with Clear-sol I (Nacalai Tesque, Inc.) and the radioactivity on the filter was measured using a β-scintillation counter (LSC-8000, Hitachi). The inhibitor used for the inhibition experiment was added simultaneously with D-[³H]serine uptake buffer.

### 2-3. Construction of FlpIn293TR Stable-Expressing Cell Line

(1) Cloning of pcDNA5-hSLC5A8 (Human SLC5A8/SMCT1)

The hSLC5A8 cDNA sequences: NM_145913 (SEQ ID NO: 1), AF536216 (SEQ ID NO: 25), AF536217 (SEQ ID NO: 26) and AK313788 (SEQ ID NO: 27) are currently usable in the NCBI database. AF536216 (SEQ ID NO: 25) is used in functional analysis research, while NM_145913 (SEQ ID NO: 1) has been identified from several genome/proteome research projects. The cDNA of hSLC5A8_AK313788 (SEQ ID NO: 27) was acquired from an independent administrative institution, the National Institute of Technology and Evaluation (NBRC, NITE). AK313788 (SEQ ID NO: 27) includes variations V193I, A201T and M490I in comparison to NM_145913 (SEQ ID NO: 1). The cDNA clone NM_145913 (SEQ ID NO: 1) was therefore constructed from AK313788 (SEQ ID NO: 27).

The cDNA of hSLC5A8_AK313788 (SEQ ID NO: 27) first acquired from NBRC was incorporated into the expression vector p3×FLAG-CMV-14, to obtain hSLC5A8_AK313788 having the 3×FLAG tag added at the C-terminus. The coating sequence of hSLC5A8_AK313788 (SEQ ID NO: 27) was amplified by Polymerase Chain Reaction (PCR) using the primers: 5′-ACTAAGCTTATGGACACGCCACGGGGC-3′ (with HindIII at the 5′-end) (SEQ ID NO: 28) and 5′-GCCGGATCCCAAACGAGTCCCATTGCTCTTG-3′ (with BamHI at the 3′-end) (SEQ ID NO: 29). The PCR reaction and thermal cycle profiling were carried out using Q5 High-Fidelity DNA polymerase (New England BioLabs), according to the manufacturer's protocol: PCR reaction was carried out with 100 ng vector template, 0.2 mM dNTPs, 0.5 μM of each primer, and Q5 High-Fidelity DNA polymerase mixed to a total amount of 50 μL. After reaction for 30 seconds at 98° C., 35 cycles each consisting of 10 seconds at 98° C., 30 seconds at 55° C., 50% Ramp and 1 minute at 72° C. were carried out. Reaction was then carried out for 2 minutes at 72° C.

The PCR product was analyzed by 1% agarose gel electrophoresis and the expected size of 1.9 kbp was obtained. The PCR product was purified using a Gel and PCR clean-up kit (Macherey-Nagel, Germany), and plasmid vector was purified using a FavorPrep plasmid extraction mini kit (Favorgen) according to the manufacturer's protocol. The purified plasmid vector and PCR product were cleaved with HindIII and BamHI, and the cleaved PCR insert was ligated with the cleaved vector using T4 DNA ligase (NEB). The vector and PCR insert were mixed in a proportion of 8:1 and reacted for 1 hour at 16° C. DH5a E. coli competent cells (BioDynamics Laboratory) were then transformed by the heat shock method. The cells were cultured on an LB medium plate containing 100 mg/L ampicillin for 16 hours at 37° C. The ampicillin-resistant clones were collected and screened by DNA size screening. The DNA size screening was carried out by mixing E. coli colonies in lysing buffer (10% w/v sucrose, 100 mM NaOH, 100 mM KCl, 5 mM EDTA, 0.25% w/v SDS, 0.05% w/v bromophenol blue), incubating for 5 minutes at 37° C., and then analyzing by 0.8% agarose gel electrophoresis. The screening method showed that the positive clones exhibited larger DNA size than the vector backbone on agarose gel. The DNA sequences of the final positive clones were confirmed. The construct was named “pCMV14-hSLC5A8_AK313788-3×FLAG”.

In the subsequent step, pcDNA5/FRT/TO (Invitrogen) was used as the vector backbone and pCMV14-hSLC5A8_AK313788-3×FLAG was used as template to produce “pcDNA5-hSLC5A8_NM145913-3×FLAG”, creating an hSLC5A8_NM145913-3×FLAG insert. Since three mutations (V193I, A201T, M490I) existed for hSLC5A8_AK313788, they were further mutated and HiFi DNA Assembly (NEB) was used for simultaneous subcloning. Before producing pcDNA5-hSLC5A8_NM145913-3×FLAG, the construct “pcDNA5-hSLC5A8_M490I-3×FLAG” (a clone with V193 and A201 mutated to I193 and T201, respectively) was created. The following three primer sets were used to prepare the PCR product.

1)
(SEQ ID NO: 30)
5′-TAAGCTTGGTACCGAGCTCGGCGCGCCATGGACACGCCACGGGG
C-3′
(SEQ ID NO: 31)
5′-AAATCCAGCCACCATGATCCCAACTTGAAAAACATCTGTCCAG-3′
2)
(SEQ ID NO: 32)
5′-TTGGGATCATGGTGGCTGGATTTGCATCCGTGATTATACAGGC-3′
(SEQ ID NO: 33)
5′-CCACCAACCATACCAAATACGCTGAGTGCTGCCTGC-3′
3)
(SEQ ID NO: 34)
5′-GCGTATTTGGTATGGTTGGTGGACCACTTA-3′
(SEQ ID NO: 35)
5′-TTTAAACGGGCCCTCTAGACTCGAGCTACTTGTCATCGTCATCCTT
G-3′

The PCR reaction was carried out using Q5 High-Fidelity DNA polymerase, following the manufacturer's protocol with the following modifications. The method was carried out with 100 ng vector template, 0.2 mM dNTPs, 0.5 μM of each primer and Q5 High-Fidelity DNA polymerase mixed to a total volume of 50 μL. After reaction for 30 seconds at 98° C., 35 cycles each consisting of 10 seconds at 98° C., 30 seconds at 55° C., 50% Ramp and 30 seconds at 72° C. were carried out. Reaction was then carried out for 2 minutes at 72° C. The PCR product was analyzed by 1% agarose gel electrophoresis and the expected size of 1.9 kbp was obtained. The PCR product was analyzed by electrophoresis with 1% agarose gel. After creating a 3-piece PCR product, it was combined with linearized pcDNA5/FRT/TO having BamHI+XhoI added using a HiFi DNA Assembly Kit (NEB), in a vector:insert ratio of 8:1. The mixture was incubated for 1 hour at 50° C. and used to transform DH5a E. coli competent cells by the heat shock method. The positive clones grown in ampicillin-resistant LB medium were screened by DNA size screening and sequenced. The final obtained product was “pcDNA5-hSLC5A8_M490I-3×FLAG” (having a 3×FLAG tag added at the C-terminus).

This pcDNA5-hSLC5A8_M490I-3×FLAG was used as template to induce mutation from M490 to 1490. The PCR reaction was carried out using the following primers to induce site-specific mutation.

5′-CTACAATGAGACAAATTTGATGACAACCACAGAAATGC-3′ (SEQ ID NO: 36) 5′-GCATTTCTGTGGTTGTCATCAAATTTGTCTCATTGTAG-3′ (SEQ ID NO: 37)

The PCR reaction was carried out using Q5 High-Fidelity DNA polymerase, following the manufacturer's protocol with the following modifications. The method was carried out with 100 ng vector template, 0.2 mM dNTPs, 0.5 μM of each primer and Q5 High-Fidelity DNA polymerase mixed to a total volume of 50 μL. After reaction for 30 seconds at 98° C., 18 cycles each consisting of 30 seconds at 98° C., 30 seconds at 55° C. and 7 minutes at 72° C. were carried out. Reaction was then carried out for 7 minutes at 72° C. After adding DpnI to the PCR product of all the plasmids, incubation was carried out at 37° C. for 2 hours and a Gel and PCR clean-up kit was used for purification. The purified plasmids were used to transform DH5a E. coli competent cells by the heat shock method. The positive clones were then grown in ampicillin-resistant LB medium and sequenced. A final pcDNA5-hSLC5A8_NM145913-3×FLAG positive clone was obtained and named “pcDNA5-hSLC5A8-3×FLAG”.

(2) Cloning of pcDNA5-hSLC5A12 (Human SLC5A12/SMCT2)

The protein hSLC5A12 was amplified from a human kidney cDNA library. The protein was designed by cloning hSLC5A12cDNA in the expression vector p3×FLAG-CMV-14 to tag 3×FLAG at the C-terminus. The coding sequence for hSLC5A12 was amplified by PCR using the following primers.

| | (KpnI at the 5′-end) |
|---|---|
| (SEQ ID NO: 38) | |
| | 5′-TTAGGTACCCATGGAGGTGAAGAACTTTGCAG-3′ |
| | (BamHI At the 3′-end) |
| (SEQ ID NO: 39) | |
| | 5′-CCGGGATCCGTAGAAATGGGTAGTCTC-3′ |

PCR reaction and thermal cycle profiling were carried out using Q5 High-Fidelity DNA polymerase (New England BioLabs), according to the manufacturer's protocol with modification: PCR reaction was carried out with 100 ng vector template, 0.2 mM dNTPs, 0.5 μM of each primer, and Q5 High-Fidelity DNA polymerase mixed to a total amount of 50 μL. After reaction for 30 seconds at 98° C., 35 cycles each consisting of 10 seconds at 98° C., 30 seconds at 55° C., 50% Ramp and 1 minute at 72° C. were carried out. Reaction was then carried out for 2 minutes at 72° C. The PCR product was analyzed by 1% agarose gel electrophoresis and the expected size of 1.9 kbp was obtained. The PCR product was purified using a Gel and PCR clean-up kit (Macherey-Nagel, Germany), and plasmid vector was prepared using a FavorPrep plasmid extraction mini kit (Favorgen) according to the manufacturer's protocol. The purified plasmid vector and PCR product were cleaved with KpnI and BamHI, and the cleaved PCR insert was ligated with the linear vector using T4 DNA ligase (NEB). The vector and PCR insert were mixed in a proportion of 8:1 and reacted for 1 hour at 16° C. DH5a E. coli competent cells (BioDynamics Laboratory) were then transformed by the heat shock method. The cells were cultured on an LB medium plate containing 100 mg/L ampicillin for 16 hours at 37° C. The ampicillin-resistant clones were collected and screened by DNA size screening in the same manner as SLC5A8. The DNA sequence of the obtained positive clone was confirmed, and it was named “pCMV14-hSLC5A12-3×FLAG”.

Plasmid pCMV14-hSLC5A12-3×FLAG was used as template for insertion into pCDNA5/FRT/TO by PCR, to produce hSLC5A12-3×FLAG. The following primers were used.

(SEQ ID NO: 40)
5′-TAAGCTTGGTACCGAGCTCGGCGCGCCATGGAGGTGAAGAACTTTG
C-3′
(SEQ ID NO: 41)
5′-TTTAAACGGGCCCTCTAGACTCGAGCTACTTGTCATCGTCATCCTT
G-3′

The PCR reaction and thermal cycle profiling were the same as for cloning of pCMV14-hSLC5A12-3×FLAG. The PCR products ending with XhoI and BamHI were cleaved with the corresponding enzymes. The insert was combined with linearized pcDNA5/FRT/TO having XhoI+BamHI added using a HiFi DNA Assembly Kit (NEB), in a vector:insert ratio of 1:4. The mixture was incubated for 1 hour at 50° C. and used to transform DH5a E. coli competent cells by the heat shock method. The ampicillin-resistant clones were collected and screened on agarose gel by DNA size screening in the same manner as SLC5A8. The DNA sequence of the obtained positive clone was confirmed, and it was named “pcDNA5-hSLC5A12-3×FLAG”.

### (3) Construction of Cell Line Stably Expressing FlpIn293TR

The cell line Flp-In TREx 293 was cultured at 37° C. in a 5% CO₂ environment, using Dulbecco's Modified Eagle Medium (DMEM) containing 10% fetal bovine serum (FBS), 100 units/ml Penicillin G and 100 μg/ml streptomycin (P/S). The cells were used to create three types of stable cell lines using the corresponding pcDNA plasmid constructs shown below.

1) pcDNA5/FRT/TO (empty vector) “FlpIn293TR-Mock” (Mock)
2) pcDNA5-hSLC5A8-3×FLAG “FlpIn293TR-hSLC5A8-3×FLAG” (hSLC5A8/hSMCT1)
3) pcDNA5-hSLC5A12-3×FLAG “FlpIn293TR-hSLC5A12-3×FLAG” (hSLC5A12/SMCT2)

To create stable cell lines, the Flp-In TREx293 cells were simultaneously transfected using the corresponding pcDNA5-plasmid construct and pOG44 Flp-recombinase expression vector (Invitrogen). The transfected cells were seeded in DMEM+10% FBS+P/S+5 mg/L blasticidin+150 mg/L hygromycin B at 1:20 ratio and cultured. The cells were subcultured 3 times at a 1:20 ratio and DNA insertion and gene expression were confirmed. The cells were periodically maintained with the same medium until use. FlpIn293TR-hSLC5A8-3×FLAG and FlpIn293TR-hSLC5A12-3×FLAG were confirmed to express hSLC5A8 and hSLC5A12 by Western blotting and immunofluorescent staining using anti-FLAG antibody and anti-SLC5A12 antibody. FlpIn293TR-hSLC5A8-3FLAG and FlpIn293TR-hSLC5A12-3×FLAG were expressed in the cell membranes.

### 2-4. Transport Activity Measurement in Stable Cell Lines

The stable cell line FlpIn293 TR was seeded in a poly-D-lysine-coated 24-well plate at 5×10⁴ cells/well (Mock) or 6×10⁴ cells/well (hSLC5A8 and hSLC5A12 cells), and after 24 hours, 1 mg/L doxycycline hyclate was added to induce recombinant gene expression. The cells were continuously cultured for 2 days and transport activity measurement was conducted at 80% to 90% confluence. In an ASCT2 knockdown experiment, the seeded cells were transfected after 12 hours with ASCT2 siRNA at 10 pmol/well, using Lipofectamine 3000.

Before transport activity measurement, the cells were washed 3 times with transport activity buffer (PBS+1 g/L D-glucose) warmed to 37° C. and incubated for 10 minutes at 37° C. in 500 μL of buffer. The transport activity measurement was initiated by addition of D-[³H]serine-containing transport activity buffer at the indicated concentration. The reaction product produced after addition was incubated at 37° C. for the indicated time. The reaction was suspended by removing the D-[³H] serine-containing transport activity buffer, and the product was washed 3 times with thoroughly cooled transport activity buffer. The cells were lysed with 500 μL of 0.1 N NaOH and incubated for at least one hour. Protein concentration of the cell lysate was measured by BCA protein assay. The lysate was mixed with 1 mL of Emulsifier Safe (PerkinElmer, MA, USA) and the radioactivity of the lysate was measured with a β-scintillation counter (LSC-8000, Hitachi).

In transport activity measurement for Na⁺, HBSS (+Na⁺: 125 mM NaCl, −Na⁺: choline chloride, 4.8 mM KCl, 1.2 mM MgSO₄, 1.2 mM KH₂PO₄, 1.3 mM CaCl₂), 5.6 mM D-glucose and 25 mM HEPES was used instead of PBS. For the inhibition experiment, a non-radioactive labeled compound was included as the uptake substrate as shown in the drawing.

### 2-5. Statistical Analysis

All of the experiments were repeated at least 4 times. The data were represented as mean±SEM, and analyzed by unpaired t test (Student's t-test). The statistical analysis software used was Prism 8.0. *P<0.05, **P<0.01.

### 3. Results

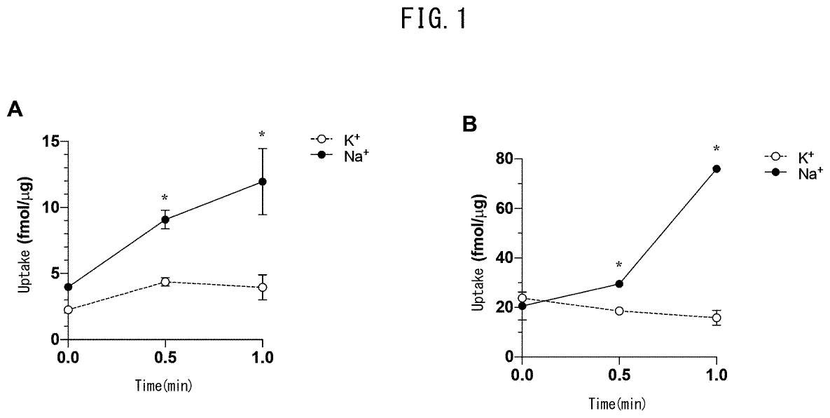

### 3-1. D-Serine Transport in BBMV was Primarily in a Na⁺-Dependent Manner (FIG. 1).

The time-dependent change in 10 μM (A) and 50 μM (B) D-[³H]serine uptake activity in mouse brush border membrane vesicles (BBMV) was measured in the presence of Na⁺ (Na⁺) and in the absence of (K⁺), and used to group the D-serine transport systems. Valinomycin was added to 5 μM to develop a membrane potential. High D-serine uptake was observed with Na⁺ uptake conditions, indicating that D-serine transport was driven primarily by Na⁺-dependent transporter.

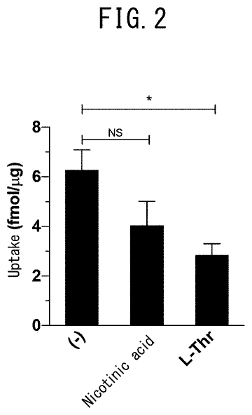

### 3-2. D-Serine Transport in BBMV was Inhibited Mainly by ASCT2 and SMCT Inhibitors (FIG. 2).

D-[³H]serine transport (10 μM) activity measurement was conducted in mouse BBMVs, in the presence or in the absence (−) of 1 mM nicotinic acid or 2 mM L-threonine (L-Thr) (measuring time: 30 seconds). Since approximately 30% of Na⁺-dependent uptake was inhibited by nicotinic acid, this suggested that SMCT transporter contributes to the effect. About 70% of uptake activity was inhibited by L-threonine, this suggested that ASCT2 also contributes to the effect.

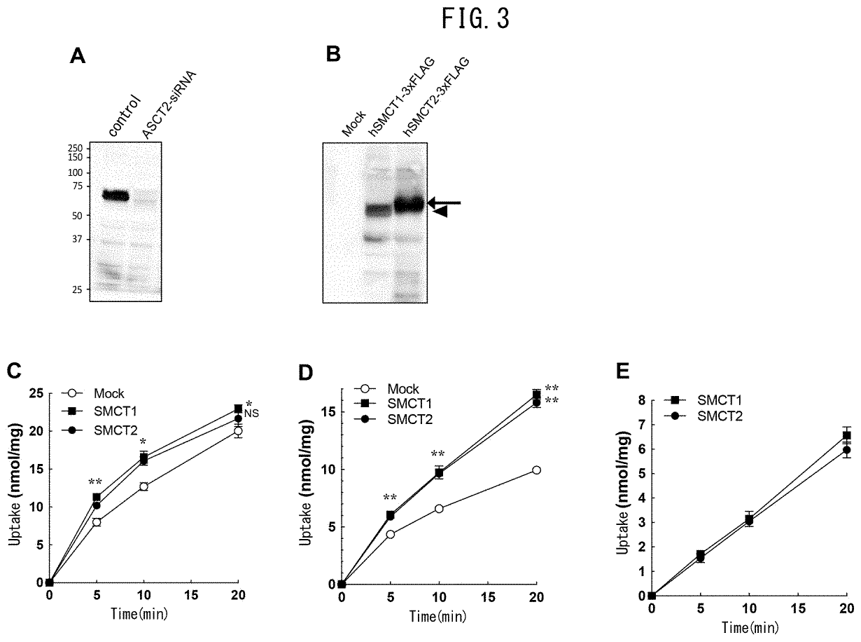

3-3. Human SMCT1 and hSMCT2 Transported D-Serine (FIG. 3).

An FlpIn293TR-hSLC5A8-3×FLAG (HSMCT1) or FlpIn293TR-hSLC5A12-3×FLAG (hSMCT2)-stable cell line was prepared, and (A) after ASCT2siRNA knockdown of Flp-In TREx 293 cells, expression was confirmed by Western blotting using anti-ASCT2 antibody. (B) Two days before the uptake experiment, doxycycline was added to the hSMCT1 and SMCT2 cells to induce expression. Expression was confirmed by Western blotting using anti-FLAG antibody. (C) The time-dependent change in uptake of 100 μM D-[³H]serine was measured for ASCT2-endogenous hSMCT1- and 2-stable cell lines. (D) Measurement was performed in hSMCT1- and SMCT2-stable cell lines using ASCT2 knockdown. (E) A graph of the observation values for Mock cells subtracted from the observation values for hSMCT1- and SMCT2-stable cell lines, based on the graph of (D).

D-[³H]serine uptake was observed in both cells, exhibiting both hSMCT1 and SMCT2-mediated D-serine transport. A significant difference was found in comparison with the control group.

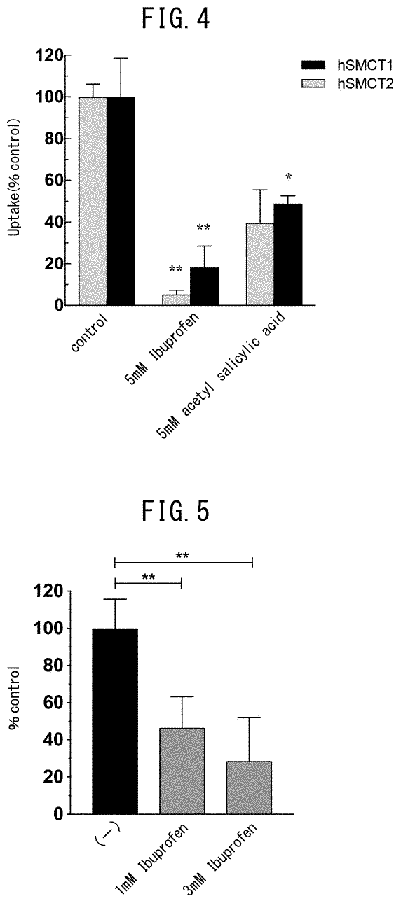

### 3-4. SMCT Transporter-Mediated D-Serine Transport was Inhibited by an NSAID (FIG. 4).

Uptake of 20 μM D-[³H]serine was measured for 10 minutes for an FlpIn293TR-hSLC5A8-3×FLAG (hSMCT1)- or FlpIn293TR-hSLC5A12-3×FLAG (hSMCT2)-stable cell line in the presence of a nonsteroidal anti-inflammatory agent (NSAID): ibuprofen or acetylsalicylic acid. Since ibuprofen and acetylsalicylic acid inhibited D-[³H]serine transport in both cell lines, this suggested that these NSAIDs target hSMCT1 and hSMCT2 transporter. A significant difference was found in comparison with the control group.

### 3-5. D-Serine Transport Activity in BBMVs was Inhibited by the SMCT Inhibitor Ibuprofen (FIG. 5).

Uptake of 50 μM D-[³H]serine in mouse BBMVs was measured for 1 minute in the presence of the SMCT inhibitor ibuprofen. D-[³H]serine transport activity was strongly inhibited with 1 and 3 mM ibuprofen.

### Example 2

### 1. Materials

The following experiment was conducted using the same materials as in Example 1.

### 2. Experiment Protocol

### 2-1. Transport Activity Measurement in HEK 293 and Flp-In TREx 293 Cells

The cell lines HEK293 and Flp-In TREx 293 were cultured at 37° C. in a 5% CO₂ environment, using Dulbecco's Modified Eagle Medium (DMEM) containing 10% fetal bovine serum (FBS), 100 units/ml Penicillin G and 100 μg/ml streptomycin (P/S). The cells were seeded in a Poly-D-lysine coated 24-well plate at 6×10⁴ cells/well. After culturing for 3 days in DMEM Medium containing 10% FBS and P/S, the cell were used for transport activity measurement. Before transport activity measurement, the cells were washed with transport activity measurement buffer (PBS+1 g/L D-glucose) prewarmed to 37° C. and incubated for 10 minutes at 37° C. in 500 μL of buffer. The transport activity measurement was initiated by addition of D-[³H]serine-containing transport activity buffer at the indicated concentration. The reaction product produced after addition was incubated at 37° C. for the indicated time. The reaction was suspended by removing the D-[³H]serine-containing transport activity buffer, and the product was washed 3 times with thoroughly cooled transport activity buffer. The cells were dissolved with 500 μL of 0.1 N NaOH and incubated for at least one hour. Protein concentration of the cell lysate was measured by BCA protein assay. The lysate was mixed with 1 mL of Emulsifier Safe (PerkinElmer, MA, USA) and the radioactivity of the lysate was measured with a β-scintillation counter (LSC-8000, Hitachi). In transport activity measurement for Na⁺, HBSS (+Na⁺: 125 mM NaCl, −Na⁺: choline chloride, 4.8 mM KCl, 1.2 mM MgSO₄, 1.2 mM KH₂PO₄, 1.3 mM CaCl₂), 5.6 mM D-glucose and 25 mM HEPES was used instead of PBS. For the inhibition experiment, a non-radioactive labeled compound was included as the uptake substrate as shown in the drawing.

### 2-2. Construction of FlpIn293TR Stable-Expressing Cell Line

An FlpIn293TR stable expressing cell line was constructed by the procedure described in 2-3. of Example 1.

### 2-3. Evaluation of Protein Expression by Western Blotting

An Flp-In TREx293- and FlpIn293TR-stable cell line was seeded on a cell culture plate at 10⁶ cells/6 cm, in antibiotic-free DMEM+10% FBS medium. On the day following culturing, it was transfected with ASCT2 siRNA at 10 pmol/well, using Lipofectamine 3000. At 8 hours after transfection, 1 mg/L of doxycycline hyclate was added to promote increased gene expression. At 2 days after transfection, the cells were washed 2 times with thoroughly cooled phosphate buffered saline (PBS: 137 mM NaCl, 2.7 mM KCl, 10 mM Na₂HPO₄ and 1.8 mM KH₂PO₄) at pH 7.4, and after collection with a cell scraper, there were centrifuged to obtain a pellet. The pellet was rapidly frozen with liquid nitrogen. The frozen cells were lysed for 30 minutes in PBS containing 1% w/v Fos-Choline-12 (Avanti Polar Lipids, AL, USA), 1% w/v n-dodecyl-β-D-maltoside (DDM; Dojindo Molecular Technologies, Japan) and cOmplete EDTA-free protease inhibitor cocktail (Roche, Switzerland). The lysate was centrifuged at 15,000×g and the protein level of the supernatant was measured with a BCA protein assay kit (Thermo Scientific). After then carrying out 10% SDS-PAGE with 50 μg (protein obtained from the lysate) per well and transferring to a PVDF membrane (Millipore, MA, USA), blocking was carried out using 5% skim milk/TBS-T (Tris buffer saline (20 mM Tris-HCl, 150 mM NaCl), 0.1% v/v Tween-20) or Blocking One (Nacalai Tesque, Inc.). The primary antibodies used were anti-FLAG-HRP (1:20,000) and anti-ASCT2 (1:2,500) antibodies, and the secondary antibodies used were HRP conjugation (Jackson ImmunoResearch, PA, USA) or StarBright fluorescent-labeled secondary antibody (Bio-Rad Laboratories, CA, USA), diluted in a proportion of 1:2,500 in Blocking One. The HRP-binding protein signal was detected by chemiluminescence (Immobilon Forte Western HRP substrate, Millipore). An image was obtained using a ChemiDoc Touch Imaging system (Bio-Rad Laboratories).

2-4. Cell survival measurement A D-serine toxicity test was carried out using HEK293 in a transient expression system. The cells were seeded in a 96-well plate at 10,000 cells/well, and transfected with 0.1 g DNA (pCMV14 as Mock, pCMV14-hSLC5A8-3×FLAG as SMCT1 and pCMV14-hSLC5A12-3×FLAG as SMCT2). At 24 hours after transfection they were treated with D-serine and further incubated for 2 days. The proliferated cell count was determined by XTT measurement. The XTT solution comprised 1 mg/ml of XTT (2,3-Bis-(2-Methoxy-4-nitro-5-sulfophenyl)-2H-tetrazolium-5-carboxanilide, disodium salt: Biotium, CA, USA) mixed with 7.5 μg/ml of Phenazine methosulfate (Nacalai Tesque, Inc.), and was added to the cultured cells. The incubation reaction was carried out for 4 hours at 37° C., 5% CO₂. The absorbance at 450 nm was then measured with a microplate reader.

A toxicity test for stable cells was carried out using an FlpIn293TR-stable cell line. The cells were seeded in a poly-D-lysine-coated 96-well plate at 6.5×10³ cells/well (Mock) and 8×10³ cells/well (hSLC5A8 and hSLC5A12 cells), while in antibiotic-free DMEM+dialyzed FBS medium. After 24 hours, they were treated with L- or D-serine at different concentrations and 1 mg/L of doxycycline hyclate was added to induce recombination gene expression. For the ASCT2 knockdown experiment, the seeded cells were transfected after 12 hours with ASCT2 siRNA at 2 pmol/well, using Lipofectamine 3000. The effect of ibuprofen was analyzed by simultaneous addition with D-serine treatment. The cells were cultured for 2 days to 80% confluence. The proliferated cell count was determined by XTT measurement in the same manner as described above.

### 2-5. Statistical Analysis

Statistical analysis was conducted by the same procedure as in 2-5. of Example 1.

### 3. Results

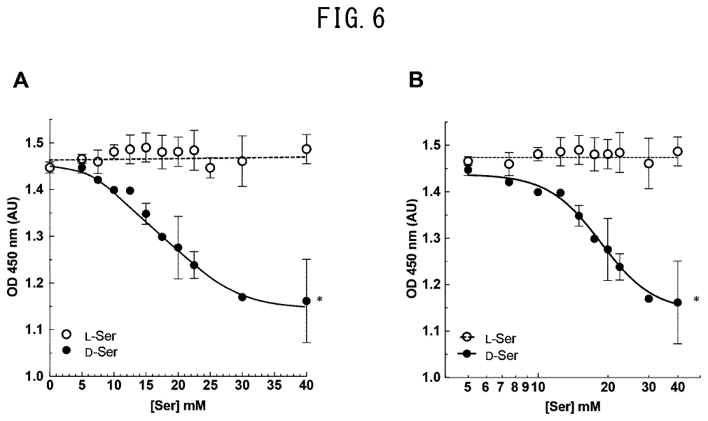

### 3-1. D-Serine Inhibited Proliferation of Flp-In TREx293 Cells (FIG. 6).

Flp-In TREx293 cells were treated for 2 days with L- or D-serine, and cell proliferation was measured by XTT assay. The same data were plotted on a linear curve plot (A) and a semi-logarithmic plot (B). The EC₅₀ for cell proliferation reduction by D-serine treatment was 18.7 mM.

The results suggested that D-serine is transported in cells via transporters, and that intracellular D-serine inhibits cell proliferation. A significant difference was seen for a D-serine concentration of 10 to 40 mM.

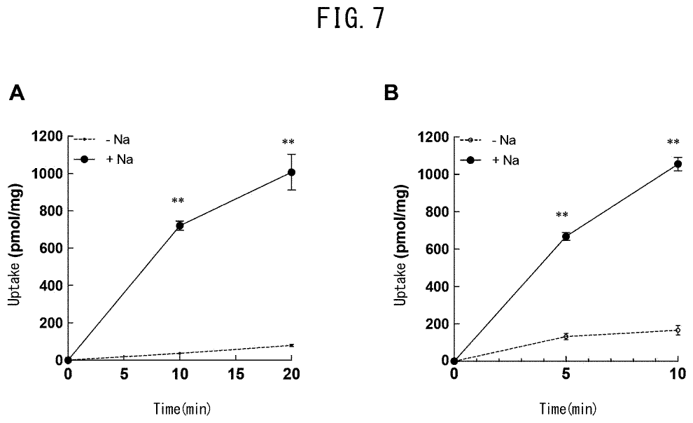

3-2. D-serine transport was driven by Na⁺-dependent transporter in the cell line (FIG. 7). The time-dependent change in 10 μM D-[³H]serine uptake activity was measured using (A) HEK293 cells and (B) an Flp-In TREx 293 cell line. In both cell lines, high uptake of D-serine was observed under the uptake conditions with Na⁺.

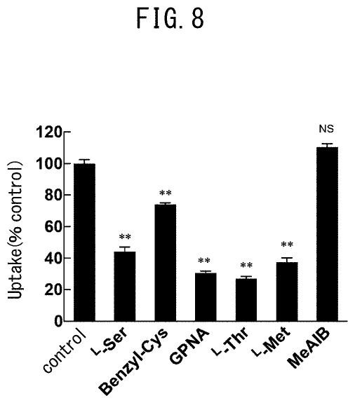

### 3-3. D-Serine Transport was Relatively Inhibited by ASCT2 Inhibitor (FIG. 8).

Uptake of D-[³H]serine was measured for 10 minutes in a Flp-In TREx 293 cell line in the presence of several inhibitors. 1) ASCT2 nonspecific inhibitors: Benzyl-Cys (S-benzyl-L-cystine) and GPNA (L-γ-glutamyl-p-nitroanilide), 2) ASCT2 substrates: L-serine, L-threonine, L-methionine, 3) system A/N inhibitor: MeAIB (α-Methylaminoisobutyric acid). Since uptake activity was inhibited by ASCT2 nonspecific inhibitors and substrates but was not inhibited by the system A/N inhibitor, this suggested that ASCT2 contributes to D-serine uptake in the cell line Flp-In TREx 293.

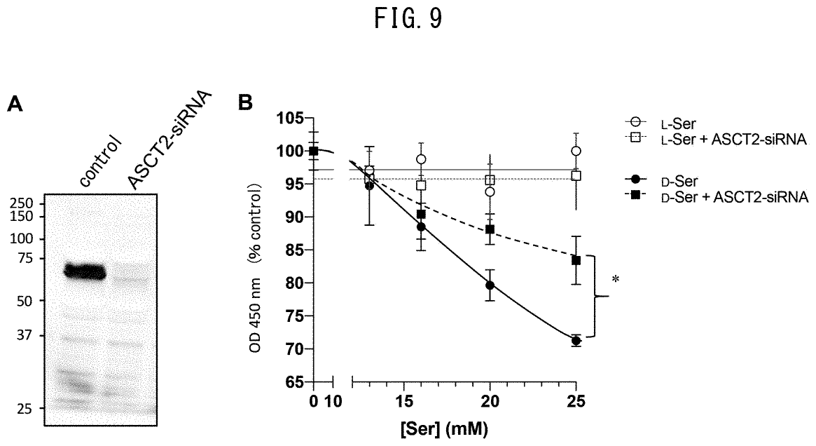

### 3-4. ASCT2 Transporter was Expressed in Flp-In TREx 293 Cells, and the KD Reduced D-Serine Toxicity (FIG. 9).

(A) Using Western blotting it was demonstrated that ASCT2 transporter was endogenously expressed in Flp-In TREx 293 cells. ASCT2 knockdown in Flp-In TREx 293 cells was carried out using ASCT2-siRNA transfection. ASCT2 expression in the membrane fraction of the Flp-In TREx 293 cells was detected by Western blotting. This suggested that endogenous ASCT2 expression is highly inhibited in ASCT2 knockdown cells.

(B) Flp-In TREx 293 control cells and ASCT2 knockdown cells were treated for 2 days with L- or D-serine, and cell proliferation was measured by XTT. L-serine had no effect on any of the control cells. However, D-serine toxicity with respect to cell proliferation was alleviated in the ASCT2 knockdown cells. This suggests strong contribution of ASCT2 to intracellular D-serine uptake.

The significant effect of D-serine in the ASCT2-siRNA-treated cells compared to D-serine in the control cells was observed with a concentration of 20 to 25 mM.

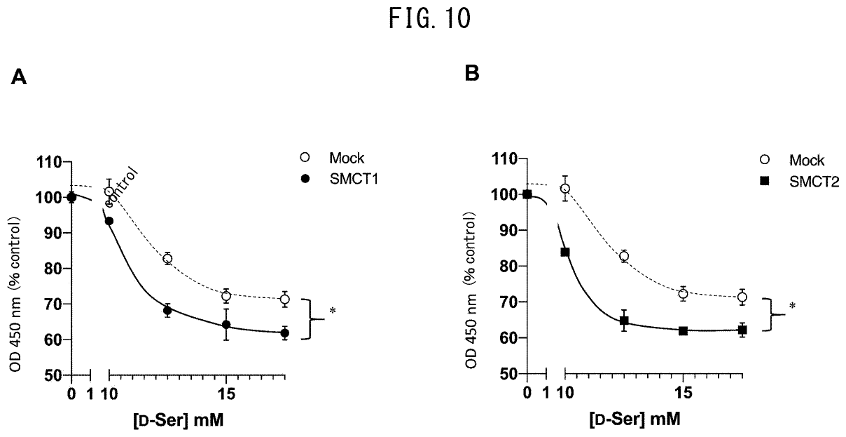

### 3-5. D-Serine Toxicity Test in a Transient Expression System for SMCT1 and SMCT2 (FIG. 10).

The D-serine toxicity test was carried out with HEK293 cells transiently transfected with (A) pCMV14-hSLC5A8-3×FLAG (SMCT1) or (B) pCMV14-hSLC5A12-3×FLAG (SMCT2). In SMCT1- and SMCT2-expressing cells, D-serine treatment induced higher cytotoxicity compared to the Mock cells (Mock).

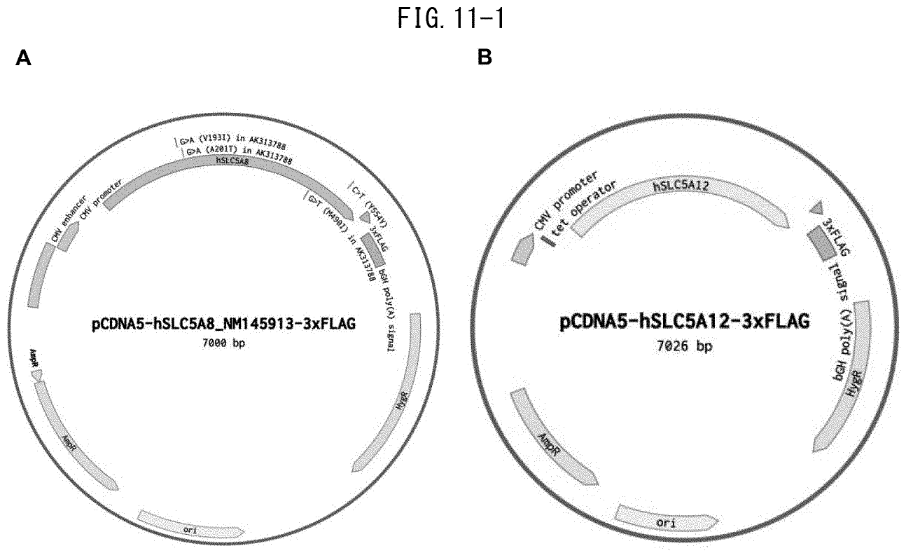

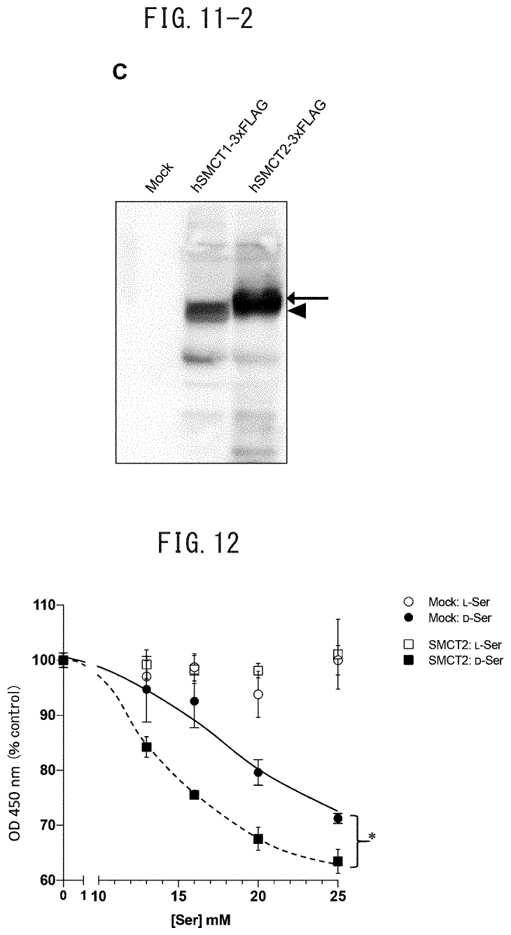

3-6. Construction of hSMCT-Stable Cell Line (FIG. 11-1 and FIG. 11-2).

(A) Vector map of pCDNA5-hSLC5A8-3×FLAG for preparation of an hSMCT1-3×FLAG-stable cell line.

(B) Vector map of pCDNA5-hSLC5A12-3×FLAG for preparation of an hSMCT2-3×FLAG-stable cell line

(C) hSMCT1-3×FLAG (arrow head) and hSMCT2-3×FLAG (arrow) were used in Western blotting with anti-FLAG antibody. This indicated that the corresponding proteins are expressed in the stable cell lines.

### 3-7. SMCT2 Augmented Inhibition of Proliferation by D-Serine (FIG. 12).

The cell proliferation effect by serine treatment was examined in an FlpIn293TR-Mock (Mock) or FlpIn293TR-hSLC5A12-3×FLAG (SMCT2)-stable cell line. The L-serine treatment did not affect cell proliferation in any of the cells. With D-serine treatment, a decrease in cell proliferation was observed in the SMCT2 cells (▪) compared to the Mock cells (●). This indicates that SMCT2 augments the proliferation decreasing effect of D-serine, and that SMCT2 contributes to D-serine uptake.

*The significant effect of D-serine in SMCT2 compared to Mock was observed with concentrations of 15 to 25 mM. P<0.05.

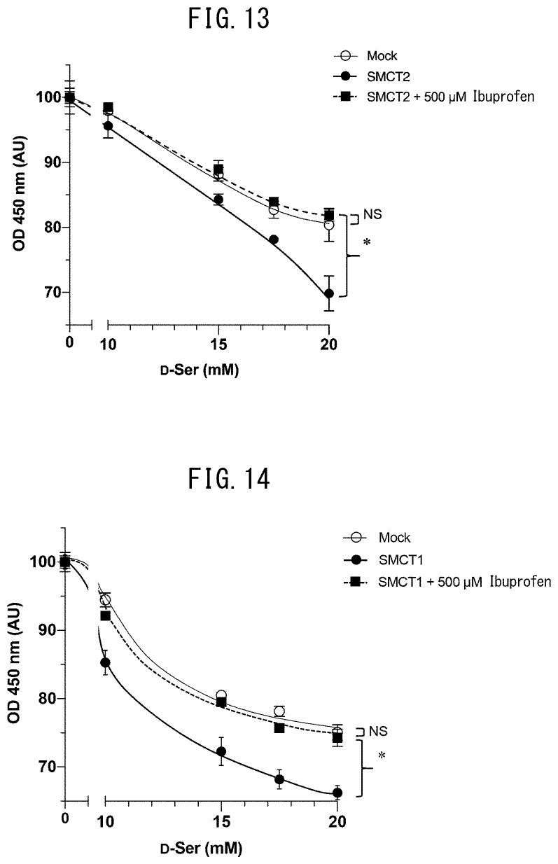

### 3-8. Ibuprofen Lowered D-Serine Sensitivity in an SMCT2-Stable Cell Line (FIG. 13).

The cell proliferation effect by D-serine treatment was examined in an FlpIn293TR-Mock (Mock) or FlpIn293TR-hSLC5A12-3×FLAG (SMCT2)-stable cell line. D-serine treatment in SMCT2 cells (●) lowered proliferation more than in the Mock cells (∘). Addition of 500 μM ibuprofen (▪) as an SMCT2 inhibitor to SMCT2 cells reduced the D-serine effect to the same level as the Mock cells. This suggested that ibuprofen inhibits SMCT2-mediated D-serine uptake. Accumulation of D-serine in SMCT2 stable expressing cells was reduced and the toxic effect of D-serine was alleviated.

The significant effect of ibuprofen in ibuprofen-added SMCT compared to SMCT2 was observed with concentrations of 17.5 to 20 mM. No significant difference was seen between SMCT2+500 μM ibuprofen and the Mock cells.

3-9. SMCT1 Increased D-Serine Sensitivity, while Ibuprofen Canceled Out the Increased D-Serine Sensitivity (FIG. 14).

The cell proliferation effect by D-serine treatment was examined in an FlpIn293TR-Mock (Mock) or FlpIn293TR-hSLC5A8-3×FLAG (SMCT1)-stable cell line. D-serine Treatment in SMCT1 cells (●) lowered proliferation more than in the Mock cells (∘). Addition of 500 μM ibuprofen (▪) as an SMCT1 inhibitor to SMCT1 cells reduced the D-serine effect to the same level as the Mock cells. This suggested that ibuprofen inhibits SMCT1-mediated D-serine uptake. Accumulation of D-serine in SMCT1 cells was reduced and the toxic effect of D-serine was alleviated.

The significant effect of ibuprofen in ibuprofen-added SMCT compared to SMCT1 was observed with concentrations of 10 to 20 mM. No significant difference was seen between SMCT1+500 μM ibuprofen and the Mock cells.

### Example 3

1. Proteome Analysis of Kidney Brush Border Membrane Fraction from IRI Mice

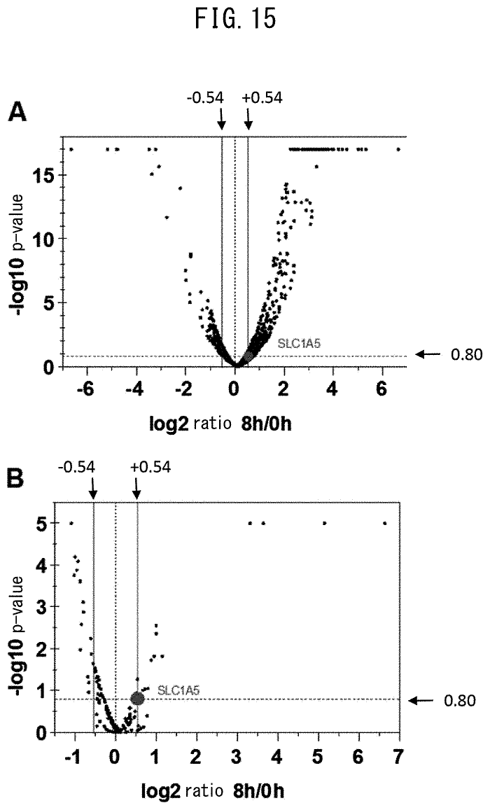

In previous research it was shown that a mouse model of kidney ischemia reperfusion injury (IRI) exhibits L-/D-serine homeostasis imbalance in the urine and serum. The low D-amino acid oxidase (DAO) activity associated with changes in kidney D-serine transport are believed to cause advanced accumulation of D-serine in the serum. This led to the idea of identifying D-serine transporter on kidney apical membranes based on expression of D-serine transporter in IRI. We conducted proteomics analysis of the kidney brush border membrane fraction taken from ischemia reperfusion injury (IRI) mouse kidneys. A volcano plot of log 2 (fold change) (8 h/0 h) with respect to statistically significant difference P value (−log 10) was drawn, from the data for all of the identified proteins (FIG. 15(A)). The transporter volcano plot is shown in FIG. 15(B). The known D-serine transporter ASCT2 exhibited a log 2 value of 0.54 and a −log 10 P value of 0.80, which was used as the cutoff value (FIG. 15). Transporters expressed in the apex membrane and exhibiting high change (either increase or decrease) in expression by ASCT2 were selected as candidates. As a result, 19 transporters were selected, among which 18 candidates were SLC and one was MFS (Table 1).

TABLE 1
Candidate transporters based on mass spectrometry of IRI mouse kidney
membrane fraction. The list is classified as SLCs or MFS, and rearranged based on the
IRI ratio at 8 h/0 h.

| | | Log2 | | | | | |
|---|---|---|---|---|---|---|---|
| | | ratio | −Log10 | | Mascot | Abundance | Abundance |
| Transporter | Reg. No. | 8 h/10 h | (P-val) | Peptide | score | 0 h^{a} | 8 h^{a} |
| Slc1a5/Asct2^{b} | P51912 | 0.54 | 0.80 | 4 | 767 | 1.36E+07 | 2.07E+07 |

Increased Slc transporter

| | | | | | | | |
|---|---|---|---|---|---|---|---|
| Slc38a2/Snat2 | Q8CFE6 | 6.64^{c} | ***^{d} | 1 | 66 | ND | 4.424E+06 |
| Slc7a1/Cat1 | Q09143 | 1.00 | 2.55 | 1 | 131 | 2.21E+06 | 5.50E+06 |
| Slc16a10/Tat1 | Q3U9N9 | 0.88 | 1.73 | 1 | 121 | 2.12E+06 | 3.56E+06 |

Decreased Slc transporter

| | | | | | | | |
|---|---|---|---|---|---|---|---|
| Slc10a2/Ntcp2 | P70172 | −1.02 | 3.75 | 1 | 18 | 6.84E+06 | 4.53E+06 |
| Slco4c1/Oatp-h | Q8BGD4 | −0.93 | 4.09 | 9 | 4011 | 1.55E+08 | 8.42E+07 |
| Slc19a3/Thtr2 | Q99PL8 | −0.88 | 3.63 | 3 | 160 | 4.26E+07 | 2.56E+07 |
| Slc2a5/Glut5 | Q9WV38 | −0.88 | 3.6 | 2 | 1140 | 6.17E+07 | 3.43E+07 |
| Slco2b1/Oatp2b1 | Q8BXB6 | −0.87 | 1.98 | 1 | 36 | 1.28E+06 | 6.76E+05 |
| Slc36a1/Pat1 | Q8K4D3 | −0.84 | 2.58 | 3 | 103 | 6.16E+06 | 3.67E+06 |
| Slc15a2/Pept2 | Q9ES07 | −0.8 | 3.11 | 9 | 2379 | 1.64E+08 | 9.99E+07 |
| Slc23a1/Svct1 | Q9Z2J0 | −0.79 | 2.88 | 21 | 17542 | 2.57E+09 | 1.82E+09 |
| Slc51a/Ost | Q8R000 | −0.66 | 1.19 | 1 | 39 | 1.26E+05 | 1.17E+05 |
| Slc6a18/B⁰at3 | O88576 | −0.61 | 2.24 | 11 | 4310 | 1.88E+08 | 1.33E+08 |
| Slc22a13/Oatn1 | Q6A4L0 | −0.6 | 1.87 | 15 | 5948 | 5.71E+08 | 3.32E+08 |
| Slc5a8/Smct1 | Q8BYF6 | −0.57 | 1.75 | 25 | 26044 | 2.69E+09 | 1.83E+09 |
| Slc5a9/Sglt4 | Q8VDT1 | −0.55 | 1.64 | 11 | 7598 | 2.96E+08 | 2.18E+08 |
| Slc22a7/Oat2 | Q91WU2 | −0.52 | 1.51 | 13 | 6100 | 4.24E+08 | 2.65E+08 |
| Slc5a12/Smct2^{e} | Q49B93 | −0.22 | 0.5 | 12 | 4725 | 4.02E+08 | 3.52E+08 |
| TMEM27^{f} | Q9ESG4 | −0.22 | 0.88 | 9 | 23686 | 2.77E+09 | 2.67E+09 |

Increased Mfs transporter

| | | | | | | | |
|---|---|---|---|---|---|---|---|
| Mfsd5/sMot2 | Q921Y4 | 0.57 | 0.86 | 1 | 26 | 3.49E+5 | 3.92E+5 |

^{a}Average of 2 measurements: n = 3 for each
^{b}Asct2 was used as the baseline for selection of D-serine transporter candidates
^{c}Protein detected only at 8 h and not detected at 0 h. The default value at 8 h/0 h with Proteome Discoverer 2.2 was defined as 100, and the log2 ratio was calculated to be 6.64.
^{d}*** p < 0.00001
^{e}Since Smct1 passed through the assessment standard, Smct2 was also selected as a member of the same family.
^{f}Collectrin is a B⁰at3-related protein which is necessary for B⁰AT3 function, and it is therefore included in the table.
ND: Not detected.

### 2. Screening of D-Serine Transporters by Cytotoxicity Test

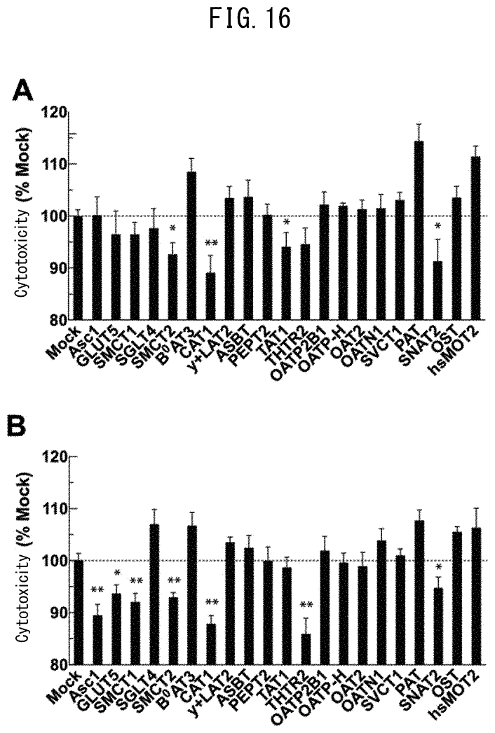

A total of 19 D-serine transporter candidates were selected from the proteome results. A cytotoxicity test for screening of D-serine transporter was developed based on the idea that intracellular D-serine accumulated via D-serine transporter function induces cytotoxicity. HEK293 cells were transiently transfected with a cDNA clone and the cells were treated with 15 mM or 25 mM D-serine to observe the initial stage and stationary stage of the toxicity effect. At 2 days after D-serine treatment, the cells were provided for XTT cell proliferation assay. The D-serine toxicity effect for each construct was compared with Mock cells. The D-serine transporter Asc1 known to be endogenously expressed in membranes was used as a positive control. The results indicated that cells transfected with SMCT2, CAT1, TAT1 and SNAT2 had increased toxicity with 15 mM D-serine treatment (FIG. 16(A)). With 25 mM D-serine treatment, significant toxicity was observed in the cells transfected with GLUT5, SMCT1, SMCT2, CAT1, THTR2 and SNAT2 (FIG. 16(B)).

### 3. Identification of SNAT2 as D-Serine Transporter

In order to establish positive candidates for D-serine transporter it was attempted to elucidate the transport function in a cell model. ASCT2 is expressed in several cell lines, constituting a strong background of D-serine accumulation and interfering with identification of novel D-serine transporters. HAP1 cells (Horizon Discovery), which are an essentially haploid cell line, can be advantageously used as a cell model for this purpose. Genetic modification is facilitated since almost all of the chromosomes are single copies, while their transcriptome analysis methods have been established and several gene knockout cell lines can be used. ASCT2-knockout HAP1 cells were therefore obtained and used as a model for D-serine screening.

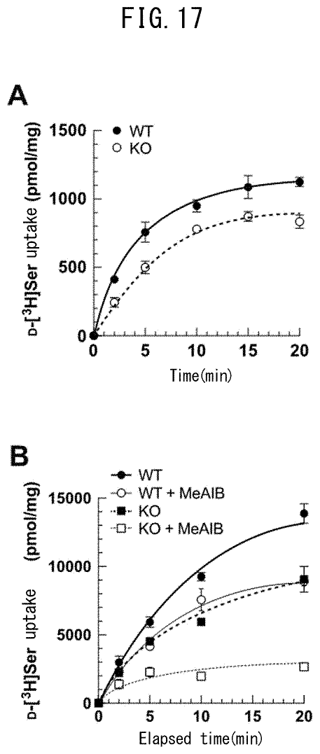

The ASCT2-knockout HAP1 cells had weaker D-serine uptake compared to wild type cells (FIG. 17(A)). However, D-serine transport background was still observed in the ASCT2-knockout HAP1 cells. Proteome analysis of the HAP1 cells (Table 2) was used to detect expression of both SNAT1 and SNAT2. SNAT1 and SNAT2 belong to the “system A family” of sodium-dependent small amino acid transporters. We considered that SNAT may contribute to D-serine transport in HAP1 cells. In order to verify this hypothesis, the inhibiting effect of the system A inhibitor MeAIB on D-serine transport was examined. As a result, MeAIB notably reduced D-serine transport in the ASCT2-knockout HAP1 cells (FIG. 17(B)), suggesting that either or both SNAT1 and SNAT2 contribute to D-serine uptake. Since SNAT2 was also detected in IRI mouse kidneys during in vitro analysis of D-serine transport by SNAT, this strongly suggested that SNAT2 is a D-serine transporter.

TABLE 2
Summary of D-serine transporter candidates. The table shows protein
expression in IRI mice, toxicity effects due to either 15 mM or 25 mM D-serine treatment,
and D-serine transporter function in ASCT2-knockout HAP1 cells, Transporters
exhibiting positive results in both the toxicity test and uptake assay were considered to be
D-serine transporters.

| | | | | | Endogenous |
|---|---|---|---|---|---|
| | | Toxicity | Toxicity | D-Ser | expression in |
| | Log2 | with 15 | with 25 | transport in | HAP1 cells by |
| | ratio | mM | mM | ASCT2-KO | proteome |
| Transporter | 8 h/0 h | D-Ser^{a} | D-Ser^{a} | HAP1 cells | analysis |
| SLC38A2/SNAT2 | 6.64 | + | + | +^{b} | + |
| SLC7A1/CAT1 | 1.00 | ++ | ++ | − | + |
| SLC16A10/TAT1 | 0.88 | + | − | − | − |
| SLC19A3/THTR2 | −0.88 | − | ++ | + | − |
| SLC2A5/GLUT5 | −0.88 | − | + | − | − |
| SLC5A8/SMCT1 | −0.57 | − | ++ | + | − |
| SLC5A12/SMCT2 | −0.22 | + | ++ | + | − |

^{a}+p < 0.05; ++p < 0.01; − no significant reduction
Uptake activity obtained by ^{b}MeAIB inhibit test

### 4. Screening of D-Serine Transporters Using ASCT2-Knockout HAP1 Cells

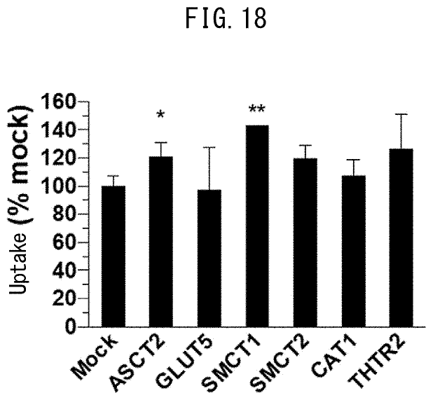

Transporter function in ASCT2-knockout HAP1 cells was examined to establish positive candidates for D-serine transporters derived from a cytotoxicity test. Cells stably expressing the candidate transporters were prepared. The cells were transfected with cDNA-containing expression vectors and maintained in selective medium. D-serine transport was measured for 10 minutes in the presence of MeAIB which reduces the background D-serine transport activity produced by SNAT2. An ASCT2-stable cell line was prepared and used as a positive control. The results indicated that SMCT1 exhibits the maximum level of D-serine uptake. SMCT2 and THTR2 had slightly increased D-serine uptake (FIG. 18).

The results of the D-serine inducing cytotoxicity test and uptake test strongly suggested that SNAT2 and SMCT1 can serve as novel D-serine transporters (Table 2). The SMCT2-positive results were not statistically significant, but SMCT2 may potentially be a different suitable candidate. Since SMCT2 is another member of SMCT that has been reported as a low-affinity transporter, it is believed that SMCT2 may transport D-serine in a low-affinity manner. SMCT1 and SMCT2 were therefore both selected for transport analysis.

### 5. GABA Inhibited ASCT2-Mediated D-Serine Transport

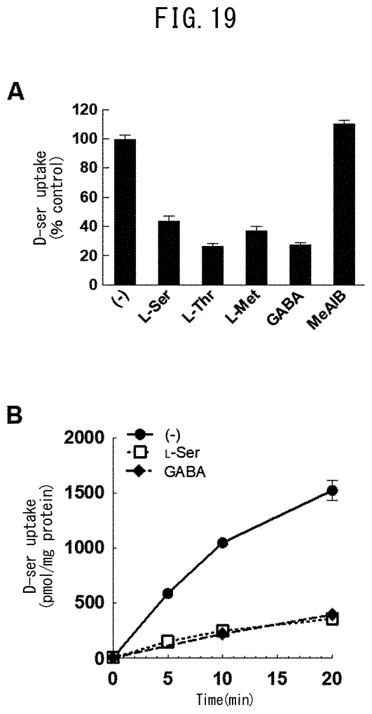

ASCT2 has been previously reported as a D-serine transporter. This previous research is consistent with the results obtained here with HEK293 cells. Endogenous expression of ASCT2 and D-serine transport function were observed in this experiment. In addition, [³H]D-serine transport was inhibited by the ASCT2 substrates L-serine, L-threonine and L-methionine, but was not inhibited by MeAIB (FIG. 19(A)). When inhibition of D-serine transport by GABA (γ-aminobutyric acid) was examined, GABA was found to inhibit D-serine transport. The inhibiting effect of GABA was further measured in HEK293 cells transfected with ASCT2. GABA inhibited [³H]D-serine transport to about the same degree as L-serine transport (FIG. 19(B)). These results strongly suggest that GABA functions as an inhibitor in an interactive manner with ASCT2.

### Sequence Listing

1. A method for treating or preventing a disease associated with an increase or a decrease in a D-serine level in a cell, in a tissue, in an organ, or in a body fluid, comprising:
administering a D-serine transport modulator to a subject in need thereof,
wherein the D-serine transport modulator modulates intracellular and extracellular D-serine transport by a D-serine transport protein,
wherein the D-serine transport protein comprises one or more selected from a first group of D-serine transport proteins consisting of SMCT family, GLUT5, CAT1, THTR2, and SNAT2.

2. (canceled)

3. The method according to claim 1, wherein the D-serine transport protein further comprises one or more selected from a second group of D-serine transport proteins consisting of ASCT family, Asc1, PAT1 and ATB^{0,+}.

4. The method according to claim 1, wherein the D-serine transport modulator modulates a D-serine level in a cell, in a tissue, in an organ, or in a body fluid.

5. The method according to claim 1, wherein the D-serine transport modulator modulates a D-serine level in blood and/or in urine.

6. The method according to claim 1, wherein the D-serine transport modulator inhibits D-serine transport into a cell by acting on the D-serine transport protein.

7. The method according to claim 1, wherein the D-serine transport modulator is selected from the group consisting of antisense RNA or DNA molecules, RNAi inducible nucleic acids, micro RNA(miRNA), ribozymes, genome-editing nucleic acids and their expression vectors, low-molecular compounds, aptamers, antibodies, antibody fragments, and combinations thereof.

8. The method according to claim 6, wherein the D-serine transport modulator is a substrate or inhibitor of the D-serine transport protein.

9. The method according to claim 6, wherein the D-serine transport modulator comprises one or more selected from a first group of substrates or inhibitors of the D-serine transport protein consisting of ibuprofen, fenoprofen, ketoprofen, probenecid, acetyl salicylic acid, naproxen, pyroglutamic acid, phenoxyacetic acid, acetic acid, propionic acid, butyric acid, L-lactic acid, D-lactic acid, pyruvic acid, nicotinic acid, acetoacetic acid, β-D-hydroxybutyric acid, β-L-hydroxybutyric acid, γ-hydroxybutyric acid, α-ketoisocaproic acid, benzoic acid, salicylic acid, 5-amino salicylic acid, 2,4-dichlorophenoxyacetic acid (2,4-D), 4-chlorophenoxyacetic acid (4-CPA), 2-chlorophenoxyacetic acid (2-CPA), 2,3-dichlorophenoxyacetic acid, 3,4-dichlorophenoxyacetic acid, 2,4,5-trichlorophenoxyacetic acid, N-(4-methane sulphonyl-2-nitrophenyl)-2H-1,3-benzodioxol-5-amine (MSNBA), fructose, N-ethylmaleimide (NEM), N-amino-L-arginine, N-amino-L-homoarginine, L-arginine, L-histidine, L-lysine, L-ornithine, metformin, chloroquine, 2,4-diamino pyrimidine, Fedratinib, AZD1480, Cerdulatinib, thiamine, methyl-amino-isobutyric acid (MeAIB), γ-glutamyl-p-nitroanilide (GPNA), 2-amino-4-bis(aryloxybenzil)amino butyric acid (AABA), L-alanine, L-methionine, L-proline, L-serine, L-asparagine, L-glutamine, L-histidine, glycine and its derivatives, and pharmaceutically acceptable salts thereof.

10. The method according to claim 9, wherein the D-serine transport modulator further comprises one or more selected from a second group of substrates or inhibitors of the D-serine transport protein consisting of phenylglycine analog, benzilserine, benzilcysteine, S-benzil-L-cystine, L-γ-glutamyl-p-nitroanilide, L-serine, L-threonine, L-methionine, L-alanine, L-cysteine, L-glutamine, D-alanine, phenylglycine analog, alanine analog, L-serine, L-alanine, L-cysteine, glycine, L-threonine, taurine, GABA, tryptophan, tryptamine derivative, 5-hydroxy-L-tryptophan, serotonin, indole-3-propionic acid, α-methyl-DL-tryptophan and its derivatives, and pharmaceutically acceptable salts thereof.

11.-13. (canceled)

14. The method according to claim 1, wherein the disease associated with an increase or a decrease in the D-serine level is a kidney disease.

15. The method according to claim 1, wherein the D-serine transport modulator enhances D-serine transport into a cell by acting on the D-serine transport protein.

16. The method according to claim 15, wherein the D-serine transport modulator is selected from the group consisting of vectors expressing the D-serine transport protein, its derivative, or a part thereof, low-molecular compounds, aptamers, antibodies, antibody fragments, and combinations thereof.

17. The method according to claim 15, wherein the D-serine transport modulator is selected from the group consisting of diclofenac, curcumin, activin A, and SMCT family-, GLUT5-, CAT1-, THTR2-, SNAT2- and PDZK1-expression vectors.

18.-21. (canceled)

22. A method of screening for a substance that modulates intracellular and extracellular D-serine transport by a D-serine transport protein, comprising:
applying a candidate substance and D-serine to a cell expressing a D-serine transport protein; and
evaluating the degree of intracellular and extracellular D-serine transport based on the expression of cytotoxicity as an indicator,
wherein the D-serine transport protein comprises one or more selected from a first group of D-serine transport proteins consisting of SMCT family, GLUT5, CAT1, THTR2 and SNAT2.

23. (canceled)

24. The method according to claim 22, wherein the D-serine transport protein further comprises one or more selected from a second group of D-serine transport proteins consisting of ASCT family, Asc1, PAT1 and ATB^{0,+}.

25. The method according to claim 22, wherein the cell is a cell engineered by externally introducing a vector expressing the D-serine transport protein.

26. The method according to claim 22, comprising selecting substances which inhibit D-serine transport into a cell by acting on the D-serine transport protein to screen for the effect of treating or preventing a disease associated with an increase in the D-serine level in a cell, in a tissue, in an organ, or in a body fluid.

27. The method according to claim 26, wherein the disease associated with an increase in the D-serine level is a kidney disease.

28. The method according to claim 22, comprising selecting substances which enhance D-serine transport into a cell by acting on the D-serine transport protein to screen the substances for the effect of treating or preventing a disease associated with a decrease in a D-serine level in a cell, in a tissue, in an organ, or in a body fluid.

29. The method according to claim 28, wherein the disease associated with a decrease in a D-serine level is a kidney disease.

30.-34. (canceled)


SEQUENCE LISTING (41 sequences)

SEQ ID NO: 1 - DNA, 4009 bp, Homo sapiens
gaacaaaacctgcccagagcgctccctgtgtagattcgctggaagcagctggaggctccagttctcatctgctcaggtgtccccggcgccttggcgaactcggccactccagttcctcacgtggtgagcactcagggcagcgggtcgattttccgaggtcccatacctgggtttgaggggcgcggctcgcagcggcgggtgcaggggcgactgccagccctcaccccgcctcggggtgcgttcggaggccgacacctggaggacgcctccagtccccgcgggacgccacgcctgcgcgccagggatccgggataagaagtgcgcgccgggctccggctgcgcgccgcggggccaccagtttgcgcgcagggctcaggcgaccgtgcggccatggacacgccacggggcatcggcaccttcgtggtgtgggactacgtggtgttcgcgggcatgctggtcatctcggccgccatcggcatctactacgccttcgctgggggcggccagcagacctccaaggacttcctgatgggcggccgcagaatgaccgcagtgcccgtggcgctgtccctcaccgctagcttcatgtcagccgtcactgtcctgggcaccccctccgaggtctaccgttttggggccatttttagcatctttgccttcacctacttctttgtggtggtcatcagcgcggaggtcttcctcccggtgttctacaaactgggaattaccagcacctacgagtatttagaacttcgatttaacaaatgtgttcgtctctgtggaacagtcctcttcattgttcaaacaattctgtatactggaattgttatttatgcccctgccctggctttgaatcaagtcacaggatttgatctgtggggcgcggtagtggcaacgggggtggtctgcacattctactgcacactgggtggtcttaaagcagttatctggacagatgtttttcaagttgggatcatggtggctggatttgcatccgtgattatacaggctgtggtgatgcaaggtggaatcagcactattttaaatgatgcctatgatggtggaagattaaatttctggaattttaatcctaaccctttgcaaagacacaccttctggacaattattataggagggaccttcacatggaccagcatctacggtgtcaaccaatcccaggtgcagagatatatttcttgtaaaagcagattccaggcaaaactgtctctctacatcaatcttgtgggactctgggcaatcctcacatgctcagtgttttgtgggctcgccctatattccaggtaccatgactgtgatccttggacagccaagaaagtgtctgcaccagaccagctcatgccttatttggtactggacattctgcaagattatccaggacttcctggactttttgtggcctgtgcttacagtgggacattaagcacagtgtcctccagtattaatgccttagcagcagtaactgtggaagatctaatcaaaccttacttcagatcgctctcagaaaggtctctgtcttggatttcccaaggaatgagtgtggtgtatggagccctgtgtattggaatggctgcgctggcgtcacttatgggagctttgttgcaggcagcactcagcgtatttggtatggttggtggaccacttatgggcctgttcgctttgggcattttggttccctttgccaactcaattggagcacttgttggtctgatggctggatttgccatttctctatgggttggaattggagctcaaatatatcctccacttcctgagagaacattgccattgcaccttgatatccaaggctgtaacagcacctacaatgagacaaatttgatgacaaccacagaaatgccatttactactagtgtttttcaaatatacaatgttcaaaggactccactgatggataactggtattctttatcatatctgtacttcagcactgttggaactttggtaacattattagtggggatacttgtcagtttatcaacaggaggaagaaaacagaacttagaccccagatacatactaaccaaagaggactttttatccaattttgatatttttaagaaaaagaagcatgttttgagctataaatcacatccagtggaagatggtggaactgataatcctgctttcaaccacattgaattgaactcagatcagagtggcaagagcaatgggactcgtttgtgaagctgctctgatactagatatccttaaatgatgtttcaattttatatgttttctaagataattggatcaggttttctttgtgtgtgtgtgtgtgttgtatcatgagtgtttgggggataagtttttgttaaaacaaagtctggactatcttcatttactacatcattaattgatgttactctggagtttagaattctggcattgacatttccctctctttcctttatttcgatgaagctataattgtgaaaattgtaactacatagatgctgaaaggctaatacacacatatgcacatgtatttgattgtcaaaggtatattcttaaatttgggtattattgaaaatattttccatgccttggtgctagcatataagtttggaagtttgccaacatcacaattcatcttgaaaagagcttttttccctcctaccacatacaccattcttagggagcaatgaggtaacaggtctgtgttgtctagatctttgctttttatccccctatcagtccagggcatatactaacctgcaaactgattctgaatcaggaaggtggtaatcaataagtattctggctgggaaagaccgtgggcccaatgatcaaagtcttcttggtgctgttcattaattcttgtgccttttggcttgttttctagagtttctgggctttggctgctgatactgcctttcttagactgtaatttttatctgcatgcccagtttctgacctatcaacttgggttttattgtgcactctaactgagcttgtcttcataattttctgtttattgccctgggcttggatatgtctcaagacactcatgtgaatcatgccaccccaaatcctggcttatcaagtcccagactataaattatgaactcccattagcttggtactaacatatacttgatgtaggtatttatggacttgatgatccaagaatattatattcttcaaaatggttaagctccatggagttagatgactacacttaatgctattaagttgaacttttgaatgtcaactaatttgcaatcaattaaagatacatatgcctagaaattttgaaatttcggtatatttatccagttaaagggctaaattatataagcaaacactactttttttaaaacgtctggactcaaaaaatgctttgttccatgttttaaaatttttaagtagcagtctcaaagttgcttagctgtttattttgctatgttcctagctaagagtttggttataggagttcatcaataacttattttttgtacagttcccacattagatactgtttaaaagttcttttttaaactcaattttttttagaaacataagagaaatatttagatacatacaaatgtttttatgattaaataattttatgcttattttctgatacgtgttatttaggtaatcatgccctgtacatttagaggttgctaactgacaatgttaagaaatttaaaaaaaaaaaaaagcctgggcatgatggctcatgcttgtaatctgaacatttgggaggctgaggcaggaagatcgcttgaggtccagagtttaagtccagcctggaaacatagttagacctcatctctacaaaaataaaaaataaaaataaaaaaaacttagctaggcatgttgccacatacttgtagtcccagttattagggaagctgaggtgggaggatagcttaagcccaggatttcaaggctgcattgagctatgattacaccactgcactccagcctgggtaacagagtgaaatcttgtctctgggaaaaaaaa

SEQ ID NO: 2 - protein, 610 aa, Homo sapiens
MDTPRGIGTFVVWDYVVFAGMLVISAAIGIYYAFAGGGQQTSKDFLMGGRRMTAVPVALSLTASFMSAVTVLGTPSEVYRFGAIFSIFAFTYFFVVVISAEVFLPVFYKLGITSTYEYLELRFNKCVRLCGTVLFIVQTILYTGIVIYAPALALNQVTGFDLWGAVVATGVVCTFYCTLGGLKAVIWTDVFQVGIMVAGFASVIIQAVVMQGGISTILNDAYDGGRLNFWNFNPNPLQRHTFWTIIIGGTFTWTSIYGVNQSQVQRYISCKSRFQAKLSLYINLVGLWAILTCSVFCGLALYSRYHDCDPWTAKKVSAPDQLMPYLVLDILQDYPGLPGLFVACAYSGTLSTVSSSINALAAVTVEDLIKPYFRSLSERSLSWISQGMSVVYGALCIGMAALASLMGALLQAALSVFGMVGGPLMGLFALGILVPFANSIGALVGLMAGFAISLWVGIGAQIYPPLPERTLPLHLDIQGCNSTYNETNLMTTTEMPFTTSVFQIYNVQRTPLMDNWYSLSYLYFSTVGTLVTLLVGILVSLSTGGRKQNLDPRYILTKEDFLSNFDIFKKKKHVLSYKSHPVEDGGTDNPAFNHIELNSDQSGKSNGTRL

SEQ ID NO: 3 - DNA, 6285 bp, Homo sapiens
attccacccccagtggttattaatttcagaacacatctgaattccttctctgtggcatatgctttaggagaggagcagacagctcttagctagggtcagatttcaaattctcatctcttggtgccaataccaccaccagattcttctttgaagtcaacttttgagatcttcactaagtacacgttggtgtctgaagattcacacgagtgcctctggtaatcattttcttcagggaatcacagtctctcctctcagcaaagcatccactgtactgaactttgcttttggaaacatcttcttcctgagacctcgttgaaagaaactctctggtgtcatactttccaatatggaggtgaagaactttgcagtttgggattatgttgtatttgcagccctctttttcatttcctctggaattggggtgttctttgccattaaggagagaaaaaaggcaacttcccgagagttcctggttgggggaaggcaaatgagctttggccctgtcggcttgtctctgacagccagcttcatgtcagctgtcacggtcctggggaccccttctgaagtctaccgctttggggcatccttcctagtcttcttcattgcttacctatttgtcatcctcttaacatcagagctctttctccctgtgttctacagatctggtatcaccagcacttatgagtacttacaactacgattcaacaaaccagttcgctatgctgccacggtcatctacattgtacagacgattctctacacaggagtggtggtgtatgctcctgccctggcactcaatcaagtgactgggtttgatctctggggctctgtgtttgcaacaggaattgtttgcacattctactgtaccctgggaggattaaaagcagtggtgtggacagatgcatttcagatggttgtcatgattgtgggcttcttaacggttctcattcaaggatcaactcatgctgggggattccacaatgtattagagcaatcaacaaatggatctcgactacatatatttgactttgatgtagatcctctcaggcgacacactttttggactatcacagtgggaggaacttttacttggctcggaatctatggggtcaatcaatcaactattcagcgatgcatctcttgcaaaacagaaaagcatgctaagcttgccttgtattttaacttgctgggtctctggatcattctggtgtgtgctgtcttctctggcttaatcatgtactctcactttaaagactgtgacccttggacttctggcatcatctcagcaccagaccagctgatgccgtactttgtcatggagatatttgccacaatgccaggactgccaggactttttgtggcttgtgccttcagtggaactctgagcaccgtggcttccagcatcaatgccttggcaacagtgacctttgaggattttgtcaagagctgttttcctcatctctccgacaagctgagcacctggatcagtaaaggcttatgtctcttatttggcgtgatgtgtacctctatggctgtggctgcatctgtcatgggaggtgttgtgcaggcttccctcagcattcacggcatgtgtggaggaccaatgctgggcttattctccctgggaatcgtgttcccttttgtgaattggaagggtgcactaggaggtcttcttactggaatcaccttgtcattttgggtggccattggggccttcatttaccctgcaccagcctctaagacatggcctttgcctctgtcaacagaccaatgtatcaaatcaaatgtgacagcaacagggcctccagtactatccagcagacctggaatagctgatacctggtactcgatctcctacctttactacagtgcagtgggctgcttaggatgcattgttgctggagtaatcatcagcctcataacaggtcgccaaagaggtgaggatattcaaccactgttaattagaccagtttgtaatttattttgcttttggtctaagaagtacaaaacactatgctggtgtggagttcagcatgacagtgggacagagcaggaaaaccttgagaatggcagtgcccggaaacagggggctgaatctgtcttacagaacggactcagaagagaaagcctggtacatgttccaggctatgatcctaaggacaaaagctacaacaatatggcatttgagactacccatttctaaggcaatacctgtatgaatgcacacacacacgtgcaatacacacacacacacacaaactccacatacttcttgcctacttgttagtagatatgtatagttgccattgctagaagacagggatgtctggtgcctatttctacttatttataactacatgcaaaatgactgtctctcgggatattctttgaaagactccaactttcacagagaaaaaccaacctgctccaaatgcccttgactacttccttcttgaataaattagggctggatttcattaccattcaagaaagcgaagtctttttgcttggtgtcatattaaacttcaggtttttcgttttagtagttttttaaccatcaaaatatcttggagtttagaggcagaacgggaaacagaaatatgcatatttaacactttcctgccacgagggataaaatagaggaatgacatccacccctgacctcatacctgacatacatgtagatatattttatgccacccatctcccatcctgtagctacaattggcatacaactactattaacctcccttcaccaccactgtcaggtcctcttccagtcattccagtcattagctgtcctgaccaaacattaaaaaaaaaattcagctaaatacagaagaagatggtatgtctggctagtgggagtgattataactaaaaactttgctccttttgtgctgtccatgcagtatgtcttcttcctttctatcactttacaatgaaaaattgcctcagagctcaataagaagtctagagcctttttccagggctaaggaaagagaaaaggaatgtcctatagaaggttgttaggatagaatttggtaaaagaacgttgcaaatattgtaacagaccataggagatttcatcagcaataggattcttctttggagaaaatacattgtccataagacttgtactctgctcattcaactcatgtgagcaagctcaactcactccacctgggttaggtaacagaagtggagaacttcatagttcgtgtctagaaaataatgtttaaagttctggagaatgagggtattgcagattaaaaggcgagttgacaaatgaaggagcagtgaaagatttttggaagaagtgaagaagtgaaattctgaaaaggtaaaagaaagaaccagtatgtcacaggggccaagtcagaggacagataataagaaacaaagttgtatctgagagtcatatattaggacaggtgtcagatatttattttggtggccagataaaagcaaaaggcctagaaacagtgtgttagcaaagtaagaagaaatggtccaaataggcaaggataaggaaatccaaaggttgtctttaaatatttctcaaaagagaaagccttgaaagaagcatacaatagagaaaaaataaattaccagtatttattattagaaaagatagaaagacagacaaatcagtggaggaattaaaacagagaaactggagtttataaaacagagcccaatccttgccttctctccctccactcaaatagaaaaggagaatggagaaagagaaagaaggtattaggctacagtttataagagagatgagaaaaaaatacatttgggaatagagggaaagggtcaaaaggggtcacatttggagaaatatctgaaaatgagaaggagcagaatttttggaaacattttttaaagtctggcaacgctaattaagctgttgatctaaggatttgcaaattgagaggtgcaattattttccaaatgatttgtgacactcttattaattagaatatatattttgtgaatattgaaatctgagccaaaactagttagctttattaatatcttagggaaagaagagagaaagaaagagggagggagagagagaaagaaagaaagaaagaaagaaagaaagaaaagaagaaagaaagaaagaaaaagacaggaagaggaagagagagacagaaagaataaaaaaacagcaattctgaggatgagaataaattaatggtaggtattacatatgcatatacatatatatatctgtacacatatatatgtaaatatgatttggaatacatcaggattgtgaacttccacttgggatagcataaaagcaaaatataccatacaatttttcattttactcacaaattttactcacaaattctcctaaagacatttcactaggtcagtacatttccgagaaagttttaaaaacgtttcttcttttatagtccctaattatcttacctggagtgttatcaaaatgtcatacaactggaaaatgcaattattattataaaaatatctctgccttctcttgtgtaatgaaatttatggcattaggagatgacatttcaagagattttagaggtcaatgtagagactccccccattcttcttaattgaacaaacatgcataaatctagggtttcaggtagagagcagatgggaatctacaggaaactgtcttccatagtgtatgaaaaccgtgtttgttgtgtggtgaagcaaaaggaaactatgaagcgcacagtcaatagattcagctagtgagtcccagcactcctttcaggccctcactaggattagcagcccagagtcatgccaaggccactggtgcagtacaaagacagttcagtgacaagttccatccataccatggaagagggttaaacttgtggctgtttttgccacagcatgaccagagacattctcaataaatggcactaggaatgatgagttttagaggggcctaaaatgttccaaacttgatactcaccactgtacctttaaagcacaaccacagatattggtaaaaatggaacagaattacctcaatacctctttaactcattctgtatgaattcattttgatgacagacattttttgtcaccatttggaataagaactacaacagcttatgattgaggtgaggaactgcagtcaaatggcttaggttcaaacacgagttctgacacaaatctaggtaattttgggcaatattttaaaatcttagtttattcaccaattaataggagtaaaaattacccctacttaatagaattattatgaagatttatataagtaaatcatataaagtgcttagcacagtgtgactggcccacatggaacatttaatacttggtctgcagtatatgaagaaaatggtgtacatgttaacttaaatggtttcattatttctcttcaggatttttaagatgtgggatcagaatatatgttactattttgagaattagaaataataagaaaaacagaatgtatacattaaaagtcatgcctaaatttccaaagagtagtttttttaatgccagagtttaaataaaatattattttccagagagtaacttttctaaaggtcctaaattctgaaaagagagaaaactactgttataaacaagtttattactaaaagatgcaattcattactatgatttattattattttgtacatgtaaaccttgataaataaacagcatctcaacccaagtttattactcattgcacagttcaaagcaactatgactctcaaaatagcaacagaaggtggggagggtcttttccctttgtttatatagtatttgctaaaaatgaactttactattttgtggctttatagggttacaccatttcattctaaagagaaacaaaaagtaaaccattggccatgtggctttcaaacagtatttcaaagagaatattactgaaatatttttgtgctgttacagtcttaatcaaacattattacaaagatcagatacggaaaaaatagaggagtctccccttaattgttccctttgaaataaattgttttaaattattacagaagttatagctgtatgtacatgcacatttgtatatttacaagtctcatgtacttaatctcaatattgcaaattagctaggtttactatagagttaactgcttttatctagcatttataatcttgtagtataacctattttcaaataactaaaatttaattgatgtcatcaatggtacacatgaaaatgatacaaatttataaactaataaaatgtaactcgtcattgatagtaaa

SEQ ID NO: 4 - protein, 618 aa, Homo sapiens
MEVKNFAVWDYVVFAALFFISSGIGVFFAIKERKKATSREFLVGGRQMSFGPVGLSLTASFMSAVTVLGTPSEVYRFGASFLVFFIAYLFVILLTSELFLPVFYRSGITSTYEYLQLRFNKPVRYAATVIYIVQTILYTGVVVYAPALALNQVTGFDLWGSVFATGIVCTFYCTLGGLKAVVWTDAFQMVVMIVGFLTVLIQGSTHAGGFHNVLEQSTNGSRLHIFDFDVDPLRRHTFWTITVGGTFTWLGIYGVNQSTIQRCISCKTEKHAKLALYFNLLGLWIILVCAVFSGLIMYSHFKDCDPWTSGIISAPDQLMPYFVMEIFATMPGLPGLFVACAFSGTLSTVASSINALATVTFEDFVKSCFPHLSDKLSTWISKGLCLLFGVMCTSMAVAASVMGGVVQASLSIHGMCGGPMLGLFSLGIVFPFVNWKGALGGLLTGITLSFWVAIGAFIYPAPASKTWPLPLSTDQCIKSNVTATGPPVLSSRPGIADTWYSISYLYYSAVGCLGCIVAGVIISLITGRQRGEDIQPLLIRPVCNLFCFWSKKYKTLCWCGVQHDSGTEQENLENGSARKQGAESVLQNGLRRESLVHVPGYDPKDKSYNNMAFETTHF

SEQ ID NO: 5 - DNA, 4085 bp, Homo sapiens
atggccaaagtgcacccagaatgtcttctctctccattcagtgcacgcgttactttggctaaaaggaggtgagcggcactctgcccttccagagcaagcatggagcaacaggatcagagcatgaaggaagggaggctgacgcttgtgcttgccctggcaaccctgatagctgcctttgggtcatccttccagtatgggtacaacgtggctgctgtcaactccccagcactgctcatgcaacaattttacaatgagacttactatggtaggaccggtgaattcatggaagacttccccttgacgttgctgtggtctgtaaccgtgtccatgtttccatttggagggtttatcggatccctcctggtcggccccttggtgaataaatttggcagaaaaggggccttgctgttcaacaacatattttctatcgtgcctgcgatcttaatgggatgcagcagagtcgccacatcatttgagcttatcattatttccagacttttggtgggaatatgtgcaggtgtatcttccaacgtggtccccatgtacttaggggagctggcccctaaaaacctgcggggggctctcggggtggtgccccagctcttcatcactgttggcatccttgtggcccagatctttggtcttcggaatctccttgcaaacgtagatggctggccgatcctgctggggctgaccggggtccccgcggcgctgcagctccttctgctgcccttcttccccgagagccccaggtacctgctgattcagaagaaagacgaagcggccgccaagaaagccctacagacgctgcgcggctgggactctgtggacagggaggtggccgagatccggcaggaggatgaggcagagaaggccgcgggcttcatctccgtgctgaagctgttccggatgcgctcgctgcgctggcagctgctgtccatcatcgtcctcatgggcggccagcagctgtcgggcgtcaacgctatctactactacgcggaccagatctacctgagcgccggcgtgccggaggagcacgtgcagtacgtgacggccggcaccggggccgtgaacgtggtcatgaccttctgcgccgtgttcgtggtggagctcctgggtcggaggctgctgctgctgctgggcttctccatctgcctcatagcctgctgcgtgctcactgcagctctggcactgcaggacacagtgtcctggatgccatacatcagcatcgtctgtgtcatctcctacgtcataggacatgccctcgggcccagtcccatacccgcgctgctcatcactgagatcttcctgcagtcctctcggccatctgccttcatggtggggggcagtgtgcactggctctccaacttcaccgtgggcttgatcttcccgttcatccaggagggcctcggcccgtacagcttcattgtcttcgccgtgatctgcctcctcaccaccatctacatcttcttgattgtcccggagaccaaggccaagacgttcatagagatcaaccagattttcaccaagatgaataaggtgtctgaagtgtacccggaaaaggaggaactgaaagagcttccacctgtcacttcggaacagtgactctggagaggaagccagtggagctggtctgccaggggcttcccactttggcttatttttctgacttctagctgtctgtgaatatccagaaataaaacaactctgatgtggaatgcagtcctcatctccagcctccccaccccagtgggaactgtgcaaagggctgccttgctgttcttgaagctgggctgtctctctccatgttggcctgtcaccagacccgagtcaattaaacagctggtcctccactttgctggttcagccttcgtgtggctcctggtaacgtggctccaccttgatgggtcaacctttgtgtggctcctggtaacataacaacaacagttactatagtggtgagatggaaggaatcaaattttgccagagaaactaacttggtggccccgacaggtcttccggggccatgggcatttgtttagagccaaattcatcctcttaccagatccttttccagaaatacctgtctaggaaggtgtgatgtcagaaacaatgacatccagaaagctgaggaacaggttcctgtggagacactgagtcagaattcttcatcctaaattattttgttagtggaaaatggaattgcttctgtgtagtcaataaaatgaacctgatcacttttcaacaagacgtgctgctgttctttgagcaagtagaaggcaggaggaagttccctgctcattcaggcagtgttttttttgtttttatttttgcttttttttttttttttaagacggagtctcgctctgtcacccaggctggagtgcagtggtgcgatctcagctcactgcaaactccacctcccgggttcacaccattctcctgcctcagcctcctgagtagctgggactacaggcgcctgccaccacgcccggctaacttttttgtatttttagtagagactgggtttcactgtgttggccaggatagtctcaatctcctgacctcatgatccgcccacctaggcccctcaaagtgctgggattacaggtgtgagccactgcgcccggccacttcaggcagtttgactgcacgagtgtcagttccttgagggcaggggcttggtcagttttgtttatgcctgaatccccagcatctcgaagagggcctctggaggctgaggtgggaggattgcttaagcctgggaagtagaggttgcagtgagctgaaattgcaccctatactccagcctgggcaacacagtgagatcccatctcaaaaaaaatttttttaattgggcatggtgacacgcctttggggtcccagctatttgggaggctgaaatgggaggatcgcttgagcctgggaggtcaaggctgccctgagtcatgattgcatcactgcacaccagcctgcatgacagaatgagaccttgtctcaaataaataaatacatgtaagattgaggaagcttccaaatgtcctcacatatgtaatgaaatcttgaggagtcttaaaatcttgagactgtggtgggggaggaagggaaaaggaacagggtcttgatatggtttggctgtgtccccacccaaatctcatcttgaattgtagctcccataattcccatgtgttctgggagggaccctgtgggagataattgaatcatgggggcaatttcccccatactgttctcatggtagtgaatcagcctcatgagatctgatggttctgtaaggggtaaaaccttttgcttggttctcattctctcttgcctgccaccaggtaagacgtgccttttgctttctggcatgattgtgaggcctccccagccatgtggaatggcgagtccattaaatctctttttctttgcaatttacccagtctctgatacatctttatcagcagcatgaaaatggtctaacacaggtctctccccaaaaatatagagagctggaggtttttcttagagatatgaataaagggtgtgagtgtgagtgtatgtgcaaaaatatggataaaaccttgaaagtgctgaatggcctgtagcaagaagtgatgtcttctcatagatgggtggcagaggctggtggccgggagagagagcagtgggacttgggagcactttttgtgggtatgtaatggagattccaccagcaacaagctaaaccactcacaagcgggcccagaggaagtaaacagaacactgtggttgaaccatccttggccttctgcagcaaggatctgcagcctggaaggggcacagaaacaaccaaagatgaaggctctcagagctgaggagaggccaggtcagaatatcagaagtcaagaaactgcagcacctctaacacaggggaggctatgaagttcatggttctggatgtctgctccctgggttcaaaccttggtcctgccacattagttgtgcaacttggaaagttacttggcttttttgtatctcagtcttctcttctgttaaatggcactaatagtaaatgtctcacatgtccattgtgagaattaaatgac

SEQ ID NO: 6 - protein, 501 aa, Homo sapiens
MEQQDQSMKEGRLTLVLALATLIAAFGSSFQYGYNVAAVNSPALLMQQFYNETYYGRTGEFMEDFPLTLLWSVTVSMFPFGGFIGSLLVGPLVNKFGRKGALLFNNIFSIVPAILMGCSRVATSFELIIISRLLVGICAGVSSNVVPMYLGELAPKNLRGALGVVPQLFITVGILVAQIFGLRNLLANVDGWPILLGLTGVPAALQLLLLPFFPESPRYLLIQKKDEAAAKKALQTLRGWDSVDREVAEIRQEDEAEKAAGFISVLKLFRMRSLRWQLLSIIVLMGGQQLSGVNAIYYYADQIYLSAGVPEEHVQYVTAGTGAVNVVMTFCAVFVVELLGRRLLLLLGFSICLIACCVLTAALALQDTVSWMPYISIVCVISYVIGHALGPSPIPALLITEIFLQSSRPSAFMVGGSVHWLSNFTVGLIFPFIQEGLGPYSFIVFAVICLLTTIYIFLIVPETKAKTFIEINQIFTKMNKVSEVYPEKEELKELPPVTSEQ

SEQ ID NO: 7 - DNA, 7343 bp, Homo sapiens
gcactgctgatgaaacctggcgccggaacccgccagccctcggcgcccattcagtccgcgcaggcaggtgtgagcagcgggtcaactacctggcaggcgcgcacgcggccgcgggctcccgctaaccgcagcctccactcctctccccgcgcgccgcgcccccgccccgccccgccccgcccggtctcgccggccgagcgtccgttggtccttgagcgcgtccgacagtctgtctgttcgcgatcctgccggagccccgccgccgccggcttggattctgaaaccttccttgtatccctcctgagacatctttgctgcaagatcgaggctgtcctctggtgagaaggtggtgaggcttcccgtcatattccagctctgaacagcaacatggggtgcaaagtcctgctcaacattgggcagcagatgctgcggcggaaggtggtggactgtagccgggaggagacgcggctgtctcgctgcctgaacacttttgatctggtggccctcggggtgggcagcacactgggtgctggtgtctacgtcctggctggagctgtggcccgtgagaatgcaggccctgccattgtcatctccttcctgatcgctgcgctggcctcagtgctggctggcctgtgctatggcgagtttggtgctcgggtccccaagacgggctcagcttacctctacagctatgtcaccgttggagagctctgggccttcatcaccggctggaacttaatcctctcctacatcatcggtacttcaagcgtagcgagggcctggagcgccaccttcgacgagctgataggcagacccatcggggagttctcacggacacacatgactctgaacgcccccggcgtgctggctgaaaaccccgacatattcgcagtgatcataattctcatcttgacaggacttttaactcttggtgtgaaagagtcggccatggtcaacaaaatattcacttgtattaacgtcctggtcctgggcttcataatggtgtcaggatttgtgaaaggatcggttaaaaactggcagctcacggaggaggattttgggaacacatcaggccgtctctgtttgaacaatgacacaaaagaagggaagcccggtgttggtggattcatgcccttcgggttctctggtgtcctgtcgggggcagcgacttgcttctatgccttcgtgggctttgactgcatcgccaccacaggtgaagaggtgaagaacccacagaaggccatccccgtggggatcgtggcgtccctcttgatctgcttcatcgcctactttggggtgtcggctgccctcacgctcatgatgccctacttctgcctggacaataacagccccctgcccgacgcctttaagcacgtgggctgggaaggtgccaagtacgcagtggccgtgggctccctctgcgctctttccgccagtcttctaggttccatgtttcccatgcctcgggttatctatgccatggctgaggatggactgctatttaaattcttagccaacgtcaatgataggaccaaaacaccaataatcgccacattagcctcgggtgccgttgctgctgtgatggccttcctctttgacctgaaggacttggtggacctcatgtccattggcactctcctggcttactcgttggtggctgcctgtgtgttggtcttacggtaccagccagagcagcctaacctggtataccagatggccagtacttccgacgagttagatccagcagaccaaaatgaattggcaagcaccaatgattcccagctgggctttttaccagaggcagagatgttctctttgaaaaccatactctcacccaaaaacatggagccttccaaaatctctgggctaattgtgaacatttcaaccagcctcatagctgttctcatcatcaccttctgcattgtgaccgtgcttggaagggaggctctcaccaaaggggcgctgtgggcagtctttctgctcgcagggtctgccctcctctgtgccgtggtcacgggcgtcatctggaggcagcccgagagcaagaccaagctctcatttaaggttcccttcctgccagtgctccccatcctgagcatcttcgtgaacgtctatctcatgatgcagctggaccagggcacctgggtccggtttgctgtgtggatgctgataggcttcatcatctactttggctatggcctgtggcacagcgaggaggcgtccctggatgccgaccaagcaaggactcctgacggcaacttggaccagtgcaagtgacgcacagccccgccccccggaggtggcagcagccccgagggacgcccccagaggaccgggaggcaccccaccctccccaccagtgcaacagaaaccacctgcgtccacaccctcactgcagccaaaggtgcaattacttgacctgcagccccagcccaccctcggctctgcagccggttctccgggccctggtcacctccagacagctgcctggccggggccactaggctgcggctggccactgtgtctcctcacttctctgaacaaagcagttcctcccctaccagctcagccccgagctgccgcagcctcaggcagaacggaggtcaccttctctccttatcttgggaaccaggccttcctcccggggactgttctgggattgaaattgtgcatactccaaactttcgcagccatcttcccgctcagccccagacacccagcaatcaagccagatgagtaccacaaaacagtgtgtccccagcagctccccaccccagagccaaatgacagtagtgcacttaaaaaggaaaatcaggcctgttgtccttctccggttgcattcagatgggtcattagggccggaccctgcctgccccttggcttctcagggctttgctctgacaccatgacagctgcccggggctgagggcagctggctccactcaaatgaggaagaagggatcactcccattagggcctgctttgcttatgcatgtgtgtgcacatgcatgtaaaccagggaccttcagctcacggcctccaggcctgggccagttcttgctgctcctgccgtctcccccgactggctgtgtcctgagtaactggaacatgagacagtatctgcaggactggccccatggtggccgagtcagaagtctgtttcctgtgagtcgccaccgttcactcagtcttgccctcccatgctttggagccagtctggtggctcctgtaaggttctcaaggctggtggcagctcagtctggggtcaggacatgtcggggtcatgcgtttctggccctgacataagctgtctggcctctctgtgacatgatgaaattgaaatcaatccacagtccatgaaattgtgacactccaccagattaagttagggcataacattaacttggaaatggccatgtcatcacccctgcggctgtcctatagctgagatgcgtgggtcgcaggggaggtgatttctaggcatattgctgtcccttttgtgtatctgtcatccggatgcttcggaccccacgcctctgcaagtgggagagacccgagcatcctccccacccccatagctccagtgcacgccacccccgtcttgcctgggtcggggcctgcggccagcaccatttcacacacactccttgtagatgggagccagaggaaacctgaacgtgggtggagcgttccactgagtctacttcaggagacagaaggcccatgctgatgggggaggaggagggatgtgggcattttggacaccaggggaaatggaaatgctgctttcaaaacttagtttcctttccatttcttcctagtctggcctttgacacaaatctggtagaaagaagcctgataaattgagggcacttgtaccctccctgtgcccccagaaggttcttggagagaagtgcaagaatttgtgaacacggcggtggagggcgggtggatggccatgggctgagcctccgtatcaggcctgctcaccttgctgggagctttattctgatctcattttgaatgttccagagggagcatcataagagcccagagctccgatttccaaagagtgatattgacatttatggagattggtgttgtaacatattttgataaatactaacttattttgttggggttttggttgtctcttgtcttaggacctggtagttatttgcttgatttttttttccgttattttctacataggcaaagagaattcgagggatagacagtctccaagaaaagtgaagtggtgggagagaattgcttttttcttttttttcttttctctagtttttctttctggctgagatttccgtgcaagacagcacccaatagactatttagagttgacatttgacattttaatgggcgccatggctcattttgtagattgagaaggtgcgtctcccctgctccaagtctcatcatgacagcgtgctgacagctgggagtctgtggccttcctcacgcagaggccttaaagctggacacagaagcacgcctaggctgggcagggatgggacccatgccccctccttagaggacgggcttcctggttaggaaaggacacgtgggggtgccttgcataatagttcactggtcaccgtgcttttatgagtagtgtttttgtgcacttgccaggggttttctctctgtgtgagaggggagtgatttaagcaatggtgtctggagtaagccttacaattttaatagactttttcttatcatatccctcatttctttccctgaaataaaaatacacacaagcaaaaaaaaaatgatagtttcacatctcttagttcccttgcccaaacaagaatattcttagttccactggccaggattttcctacatagtcagaacttacacattactagaggcacacccaccaaggagtattgtgtctacttttatctgtgcaccagccacaaatacccacattggaaagacccatttgtgatgggtaaacatcccttcctgtctcccacaacccctgtgactgccctgcatgtgttcatgacctccgaaggcccaaattcatgaagcagcaaacccagcagatctccacccccctgcctcaggacctctgctgaagagggggatgaagtgggtctccagggaggcagtgggggccttgttggcagctggctcgggagccggcttacaggagggcagctctgcagttgggaggggcaccgtccggaggagaccaggcctctacacaccccccactctacttatcatccctgctcacacacccttgtccaaggctttatgcatcggatttatttttccaaatcaagaggacagtgatagatgcattttccccaggctgtctcagaaaggtcgctaaatgtatactgttgtcagaattgctgagatctccccccacttttggtttttgcagcagtaaaaactctttccactgtgacttattttctctctcaggcagccagccacctggtcccttgtgctgactctagcacagtggccaggatccaatacgagtccaggggtgaccgcaggatggtgggggcagcgggcttctccacctaccccagccaccaaggccctgacgcactgcctcctgcaccttcagcacatccctgtgcacagctggaagggtgcatggcccgctcacctttgttcagatgggtggaaacgctgatgataccagctcctccctgccgtgcccctgccacggagcaggcattgtgaactggctggtgtttgcagtcccacgtggcatggcctccagcccaacccacagtggagactggagacagggcaatgagtctggtggggggcacgtggacatgccccataggggccccacccagacttaacaggcaaggtcctgggcattgcgcgacgcaggactcaatgctaaagcaagcctgcctggctctgtgccagggcccctcttctgattcacacatcccatttttacacagacccttccttcttaataaaggctgacagttctgttggcagccaagaacccacaccatgaagacagggagtgaggggcctttgtgcccaactccagcacagctgcgttctggggtgtgtgagaggcatgttcgtgtctgtgcgctggtggtctcgtgagacagttccgaggacggggaaattgcagggtggtgggggcgtgaggcttatatgtggaactgatgcagagttcgcctgcagacggatctggatatacactatgtataattgttacgtgtaatttaaaatatatctgtttgccatcgtcatgagaagattatatgtaaggctctgaagggagagggagatgtacattctgccaggctcctggggaccttatccgagtcatgaaattgattactgttgatccagtggtgcaagaagctacactccatgtgtcatcacgcttatgactcctaatgtatttttaaggcaaaaaatgtcagccgactccatcttcacccctcgattcctcgagtccagcctttctgtgccagtgcttcactgagccacaacgctctcgccatcgggacccggctgggcctggagtctcggggcacagttgccatggagccctcctgggtcattctacaaatgtgctgagtgccagctgaaaaccccacaggagatggagtaccttggccaagcttaaagagaagattttctcagggtatttattagtgtgtccagcagggtcaggaagcaggatggaaagatgcactcagactgttaatttattaacaaggcaaatgattttgtgtttcttgatgacagactattaagtttgggacttattttcccatttgagaagttataatatatatttaagatgataagtttcctgcttaagttgtgcctttcagcttcaatgagtttaaggagcactaagggtaatgataccaatgagggttggtttattatcaaacctgaatagctgtggtttctccagtaaatattttcttctactgaacatggagccattattaagagttgtgtgttttttattatgtacatttgtatatttttttgcttgtttgatgttctatttttctaatagttttcttttagtttcttaaagttgtgatactagatttagattctgatgctaactgcaaatcaggttggtctctgctgggtctctcctgcttttattttactttaaggacaagtgtagttgtcgtccaccacctttcaaaaaatgtgaaactgccctgcctcccctttttgctgacaacactgtgtacattgaccacttcctaccatactttatgttgtaaaatcaaactcttttgtggtacattatctcatgcttctgcaaattcgaataaattctatggcttcca

SEQ ID NO: 8 - protein, 629 aa, Homo sapiens
MGCKVLLNIGQQMLRRKVVDCSREETRLSRCLNTFDLVALGVGSTLGAGVYVLAGAVARENAGPAIVISFLIAALASVLAGLCYGEFGARVPKTGSAYLYSYVTVGELWAFITGWNLILSYIIGTSSVARAWSATFDELIGRPIGEFSRTHMTLNAPGVLAENPDIFAVIIILILTGLLTLGVKESAMVNKIFTCINVLVLGFIMVSGFVKGSVKNWQLTEEDFGNTSGRLCLNNDTKEGKPGVGGFMPFGFSGVLSGAATCFYAFVGFDCIATTGEEVKNPQKAIPVGIVASLLICFIAYFGVSAALTLMMPYFCLDNNSPLPDAFKHVGWEGAKYAVAVGSLCALSASLLGSMFPMPRVIYAMAEDGLLFKFLANVNDRTKTPIIATLASGAVAAVMAFLFDLKDLVDLMSIGTLLAYSLVAACVLVLRYQPEQPNLVYQMASTSDELDPADQNELASTNDSQLGFLPEAEMFSLKTILSPKNMEPSKISGLIVNISTSLIAVLIITFCIVTVLGREALTKGALWAVFLLAGSALLCAVVTGVIWRQPESKTKLSFKVPFLPVLPILSIFVNVYLMMQLDQGTWVRFAVWMLIGFIIYFGYGLWHSEEASLDADQARTPDGNLDQCK

SEQ ID NO: 9 - DNA, 5213 bp, Homo sapiens
aagagcgcctggatccatccgacagccgcgggtgcgtggtcgtgcggcaagtgagcgatttggtgaacagacactcccttctgtagccatggattgttacagaacttcactaagcagttcctggatttaccccactgtgatcctctgcttatttggttttttctccatgatgagaccctcagaaccattccttatcccatatttatctggaccagataaaaacctgaccagtgcagagataacaaatgagatcttccccgtttggacatactcctacctggtgctgctgctgcctgtgtttgtcctcaccgattatgtccgctacaagccagtcatcatcttgcaaggtatcagtttcatcattacctggctgctgctgttgtttggccaaggagtgaagaccatgcaggttgtagagttcttctatgggatggtcaccgccgccgaggtggcctactacgcctacatatacagcgtggtcagccccgagcactaccagagagtgagcggctactgcaggagcgtcacgctggccgcctacacagcagggtcggtgctggctcaactcttggtatccctggcgaacatgtcgtacttttacctcaacgtcatatccttggcctctgtctccgtggctttccttttctcacttttcctaccaatgcccaagaaaagcatgttttttcatgcaaaacccagcagagaaataaagaagtcatcaagcgtgaatccagtattagaggaaactcacgaaggtgaagcaccaggctgtgaagagcagaaacccacatcagaaatactcagcacttcagggaagctgaataagggccagctgaacagcctgaaaccaagcaatgtgactgtggacgtttttgtgcagtggttccaagatttgaaggagtgctactcctcaaaacgtcttttctactggtctctatggtgggctttcgccacagcaggttttaaccaggttttgaactatgttcaaatcctgtgggattacaaggcgccatcccaagattcttccatctataatggggccgtagaagctattgcaacctttggaggggctgtggctgcctttgcagtgggttatgtgaaagtcaactgggaccttctgggagagctggctctggtggtcttctcagttgtcaatgccggttctttatttctcatgcattacacagccaatatctgggcgtgctatgctggctatttgatattcaagtccagctatatgcttcttataaccatagcagtatttcagattgcagttaatctgaatgtggaacgctatgccttggtatttggaatcaacacctttattgccttggtgattcagaccatcatgactgtgattgtagtagatcagagagggctcaacttgccagtcagcattcagtttttagtttatgggagctattttgcagtaattgctggaattttcctaatgagaagcatgtatattacctactcaaccaaatcccagaaggatgtacagagccctgctccaagtgagaatccagatgtgtctcacccagaggaagagagtaatatcatcatgtcaacaaaactctaacctcatcgcaacaaacgcaacagtggctttcaaagttatgcaataataaggaaagattttgagatgggtggcatatgttttgccataacttgacatgctttgcaaatctggattccaatggacctttcaaaaccacaacaaaacctcagttttagatgagttctctatgtgaccaattttactggatgcaattgacaggacccgtcatcataattaaacaacccatattggggaccccctgtgactagtagcagctggaaaattctggtttttatcacttgtaaagacatgcagatggcgtggaaccaaaccatgagaaaactccagccatcctggagttgatattcaccatttgtgaggagaaatactaactggactgaccctattgctaggcttaaatacttatttgatcttaccaaagaagtcaacacatgggacctttgtgtcacatgaaccattttctttcctcttctattaagtgtatttctgtttaagttacagttctctaagagaattacaatgtttgtcccatttctaagggcttctcttcaactctaataacagcattattcacgttatgatttgatagtattattatttaatttttttatgattattttccattttgtgctctgagttttgctgttgaaagtctccctcaagaatagcttcagatcctcttgtgtatttgcagaatacacaaggtcatttcccagtggcctgggagaggcagtgagccttctctccaccaccatagacaggtgttaatgcatctatgggccaggtgcagtggctcactcctgtaatccgagcaatttgggaggccaaggtgggagttttgcttgaggccagaggttcaagaccagcctgggcaacacagtgacacctgactctactaaaaaattaaaaaattagctgggcagcgtggtgtgtgcctgtagtctcagctacttgggaggccatggtgggaggatgggttgagcacaggagtcagaggctacagtgagctatgattgcaccattgcactccagcctgggcaacagagtaagactctcatccccctcctcccaaaaaagagcacctgtgtggtgtctgtctaaaataggaaaacttaaaggaggtttctagaagtattagaaataagaaaacactgatggggggagtggaataaaacacctgattttgttttgttttggtttttgagacagtctcgctctctcacccaggctggaatgcaatggcgtgatcttggctcactgcaaacttcacctcctgagttcaagccattctcctacctcagcctcccaagtagctgggactatgggtgcacaccaccacgcctggctaatttttgtatttttagtggagatggggtttcacaatgttggccaggctggtctcaaactcctgacttcaggtgatctgtctgcctcggcctcccaaagagctgagattacaggcgtgagccaccatgcctggccaaaacacctgatttttaaatggtagcaattgtgctaaataaaatgtgtaaaaattttaaagagttgttaacattttttgatgaattctgctgaaaattatctgaacatacctgtttttcgaaacttcaagaaacttcaataagggatgcattgagcagtctgacagtgagaataaagaatgctgagtcaagatttatattaaggctggaagctgtggctcacacctgtaatcccagcattttgggaggctgaagtgagaggttaatttgagggtaggggtatgagaccatcttggccaatatagcaagaccctgtgtctaaaacaaacaaaaaaatttttaattggattttgatatggtttggatctgtgtccccaccaaatctcacgttgaagtgtaatccccaatgttagaggtggggccggctgggaggcgactggatcatgggtgtgaagccttcatgaatggtttggcaccgtcccctcagtgctggtctcatgacagtgagttatcgtgagatctggtagtttaaaagtgtgtagcacctccccactctctctttctcctgctttggccacgtgaagatgcctgctcccactttgccttctgccatgagtaaaaattccccgaggactccctagaagcagatgttgccacgcttcctatatagcctgtgagctaattaaaccccttttcttacaaactatctagtctcaggtgtttctttatagcaatgtgataatggactaatatgtttctcaagtatactcctctccaaatcccgaaattcttcactggactaagatccagcccaagaatcaatgcactcacttagtggagaaaactaataacattcctacttacattaaaggcaaggaaggaatggccgctattctttttcttcttcttctttttttttttttttttttttttttgatagaatcttgctctgtcacccaggctggagtgcaatgacgcaatgtcagctcactgcaacctccgcctcccgggttcaagcaattctcctgcctcagcctcccgagtagctgggattacaggcgtgcaccaccatgcccggctgatttttgtatatttagtagagacggggtttcaccatgttggtcaggctggtttcaaactcctggtctcaggtgatccgcccgtctcggtctcccaaagtgttgggattacaggcttcagccacctcacacccagtctattatccttcttttttaacttattattctggaggcattaggcatcacaattagacaaacaagaaaaatcagaggtcagaaattggaaaggaggccaggtgcagtggctcacgcctgtaatcccaacactttgggaggctgaggcggtcagatcacatgaggccaggaattggaggcaagcctgggcaacatgatgaaaccctgtatgtactaaaaacaacaaaattagccgggtgtggtccatgcctgtaatcccagccactcggtggctgaagcataagagtcacttaaacccaggaggcagaggttgcagtgagttgagattgtgccactgcactccagcctgagcgacagagtgagacccaaagtggattaaaaaatgcattatgattccttcacatattcacctataacaaaattaactgctaaaaaatgaggaagatctttagtgacacagaactctaagagctctactatcaagtggaaaaaagtcaagcaccaaaataataatgaacgcagcaactcagcatttgtataaaaaggacaaaaatatctgtattggcttatatatgcgtataacttttctaaaaagacaaatgaggcctggcacggtggctcatgcctgtaatcctggcactttgggaggctgaggtaggtggattgcttgagttcaggagttcaaaaccagcctaggcaacatggcaaaaccctgtctctacaaaaaatacaaaaattagctgggcatggtggcttgcacctggactcccagctacttgaggggatgagaagggaggatcgcttgagcccaggaggtggaggttgcagtgagctgagatcatgccactgcactccagccctgatgacagagtgagaccctgtcttgaacaaacaaaccaacaatcaaacaaataaataaacaactgaaactcataccaa

SEQ ID NO: 10 - protein, 496 aa, Homo sapiens
MDCYRTSLSSSWIYPTVILCLFGFFSMMRPSEPFLIPYLSGPDKNLTSAEITNEIFPVWTYSYLVLLLPVFVLTDYVRYKPVIILQGISFIITWLLLLFGQGVKTMQVVEFFYGMVTAAEVAYYAYIYSVVSPEHYQRVSGYCRSVTLAAYTAGSVLAQLLVSLANMSYFYLNVISLASVSVAFLFSLFLPMPKKSMFFHAKPSREIKKSSSVNPVLEETHEGEAPGCEEQKPTSEILSTSGKLNKGQLNSLKPSNVTVDVFVQWFQDLKECYSSKRLFYWSLWWAFATAGFNQVLNYVQILWDYKAPSQDSSIYNGAVEAIATFGGAVAAFAVGYVKVNWDLLGELALVVFSVVNAGSLFLMHYTANIWACYAGYLIFKSSYMLLITIAVFQIAVNLNVERYALVFGINTFIALVIQTIMTVIVVDQRGLNLPVSIQFLVYGSYFAVIAGIFLMRSMYITYSTKSQKDVQSPAPSENPDVSHPEEESNIIMSTKL

SEQ ID NO: 11 - DNA, 4795 bp, Homo sapiens
acagccgcgcgacgccgccgccttagaacgcctttccagtactgctagcagcagcccgaccacgcgttaccgcacgctcgcgcctttcccttgacacggcggacgccggaggattggggcggcaatttgtcttttccttttttattaaaattatttttcctgcctgttgttggatttggggaaattttttgtttgttttttatgatttgtatttgactgagagaaacccactgaagacgtctgcgtgagaatagagaccaccgaggccgactcgcgggccgctgcacccaccgccaaggacaaaaggagcccagcgctactagctgcacccgattcctcccagtgcttagcatgaagaaggccgaaatgggacgattcagtatttccccggatgaagacagcagcagctacagttccaacagcgacttcaactactcctaccccaccaagcaagctgctctgaaaagccattatgcagatgtagatcctgaaaaccagaactttttacttgaatcgaatttggggaagaagaagtatgaaacagaatttcatccaggtactacttcctttggaatgtcagtatttaatctgagcaatgcgattgtgggcagtggaatccttgggctttcttatgccatggctaatactggaattgctctttttataattctcttgacatttgtgtcaatattttccctgtattctgttcatctccttttgaagactgccaatgaaggagggtctttattatatgaacaattgggatataaggcatttggattagttggaaagcttgcagcatctggatcaattacaatgcagaacattggagctatgtcaagctacctcttcatagtgaaatatgagttgcctttggtgatccaggcattaacgaacattgaagataaaactggattgtggtatctgaacgggaactatttggttctgttggtgtcattggtggtcattcttcctttgtcgctgtttagaaatttaggatatttgggatataccagtggcctttccttgttgtgtatggtgttctttctgattgtggtcatttgcaagaaatttcaggttccgtgtcctgtggaagctgctttgataattaacgaaacaataaacaccaccttaacacagccaacagctcttgtacctgctttgtcacataacgtgactgaaaatgactcttgcagacctcactattttattttcaactcacagactgtctatgctgtgccaattctgatcttttcatttgtctgtcatcctgctgttcttcccatctatgaagaactgaaagaccgcagccgtagaagaatgatgaatgtgtccaagatttcattttttgctatgtttctcatgtatctgcttgccgccctctttggatacctaacattttacgaacatgttgagtcagaattgcttcatacctactcttctatcttgggaactgatattcttcttctcattgtccgtctggctgtgttaatggctgtgaccctgacagtaccagtagttattttcccaatccggagttctgtaactcacttgttgtgtgcatcaaaagatttcagttggtggcgtcatagtctcattacagtgtctatcttggcatttaccaatttacttgtcatctttgtcccaactattagggatatctttggttttattggtgcatctgcagcttctatgttgatttttattcttccttctgccttctatatcaagttggtgaagaaagaacctatgaaatctgtacaaaagattggggctttgttcttcctgttaagtggtgtactggtgatgaccggaagcatggccttgattgttttggattgggtacacaatgcacctggaggtggccattaattggcaccactcaaactcaaactcagtccatctgatgccagtgttgagtaaactcaactactatgaaatttcacctaatgttttcagtttcacttccttttgaagtgcagattcctcgctggttcttctgagtgcagaataagtgaacttttttgttttgttttgtttttttaagaaacttatctgtatgttagaaatggatatgaacaacaaaaccacgagtctcgggttaagggaagtgacaattttattccattccagagaatggacaaactcttaacttttatcaagccacatgcttggctgtgtcattgtttaacttggatattttatgattttacttgaatgtgcctaatggaaccatttgatgtgagaaacaattctttttaatttacagcaaaatattgaataaccattgacaaaaacactattattttttgtaccaaaaatacttaaagacctcagaagcactcttttacttttaagaaattgcttttttgaactttattcagaagcagttatcaataaattccataaaataatgtcattggtatttaaaaatgaatattaatataatgaaatggtttgcctttttgtaggcataataagccaaatacttttttacccaaaataatttttagagaaaatgatgtaatgaaaaattgtaccatgaattaggagcatagttttttccatttaaacgtcaccattacttaaaagatgattgattattgctataccaaatcagatgaactctgttcatcacttttcttctctgtccccaaacaatttggttcattcagactgaaatgtttgtgtcttcaacttattagaatggaagataatgcagatatttctgtgggaaataaaataactaattttgaggtaccaaatagtgcaattgggtaaaacagggtttattcagttgcatctgtctccagtgttgtattgacagctctgggtctttttttgggccagcccttttttgacattgcttccagcagtggaaaatgggcatttgatggcaataggccaaaattattgtgtccagagagtacactttttcaaaatgctcacctactggaagtgtgaattacttgacaatgtatggcttagttgtgttcatgttttgtctacagtagaggtctaatccacaggttacacctatgtttgatatgatataagttctctttgcgtaggccactgggtttctcatgcagtaagctttataaaaactcatttgcactggactgtcatctcattcttgtacaacgtagaattacttgtttacatccaacaaatggttagctagggaaaacagtgcaaactgagtgttagtagtcattttggtccaactgcatgtcaacccttccatttcaatcccagttagaaatgaaaataattactttgaaacttggctttaagagcacatttatcgtacgtcacagtgtatggtgaatatattattaaataatgtggtacttcgctcatcaggcataatgtctaaaatctaatatacataattccattaagtggttgaaggaagcaaataatggaattgtcaattggtcatctggctgtaaggtttgcccttgaactaaaaatgttgtttggggcaagggccagaaatgtggagacatggtttttgttacgcattcttgtattatatgtgactaaatttacaaacaagatacatgtgtaattaaagacccttatggaactggaagacgtcttgtagtgctacattgggtgaaaccgttggtccatttttgtctgtttctatgaagataaaataattgggggccatctagaaatagaaaggcagtgggaagacagattctacggcactgctttcatttaattgggctttaggcactccattcgaatgcagaacctcacctctagttgagaccaagaattggcaaatttgcatgagctcctggaaagagttgctgactttgtatctaagacctgccagggaataccaagagttgtttctacagacttttttttttttttttgtatgggagaagatactgtggcaaccaggaaggaatggaaaaaaaattcttttctctacagcaaattaatgtgaggaagctcctccaatcctctggctatttaaggttcaaaatcaagtgcctagggaaaattccaatggatgattttctgggagctatcttgtctaccttgaggttcctgaacaatgaattcccattaatgagcagtcttcagtattaaaaccactgtcttgtcacctcattttgcattactgtcttccgtggatgtttcagttacaactgtaatgttatttatagaacaacattaatccattaaagctaacctatttttcaatatttatgataatctatgtacatatattgtctgtccatatgtatttgtaaataggttgtatataatgtcaggtttgggtcttgggttcaagtgtatatattcctgtaagtttcttaactgcattttgatgaattcacattatgtaactataagaattgtcccaaaagtacctgtacagaaaattgaatattgaaaaattgacaaattgtgtacaaacactaaaaaaaacttgtttaaattgtatttgcaataaacaacatcaaattttttcatgaaatcttggtacaaattcagatctcttatttaaaatttaaataaggaatacattttcaaaatgcagtaatcaaaatgtgatctagtgtaatgaaataaaatgtgatctagtgtaatggaagacctttgagaacctgggtgtattaactttgtgtatatagtgtaaatatccccactgtactgttagaggccaacaattctagtatggcttgttggcaaagagtgctacaccgtttcaatgaaacaatgtatgtttgttttaactgaactaaaataaatacatgcttaatcctga

SEQ ID NO: 12 - protein, 506 aa, Homo sapiens
MKKAEMGRFSISPDEDSSSYSSNSDFNYSYPTKQAALKSHYADVDPENQNFLLESNLGKKKYETEFHPGTTSFGMSVFNLSNAIVGSGILGLSYAMANTGIALFIILLTFVSIFSLYSVHLLLKTANEGGSLLYEQLGYKAFGLVGKLAASGSITMQNIGAMSSYLFIVKYELPLVIQALTNIEDKTGLWYLNGNYLVLLVSLVVILPLSLFRNLGYLGYTSGLSLLCMVFFLIVVICKKFQVPCPVEAALIINETINTTLTQPTALVPALSHNVTENDSCRPHYFIFNSQTVYAVPILIFSFVCHPAVLPIYEELKDRSRRRMMNVSKISFFAMFLMYLLAALFGYLTFYEHVESELLHTYSSILGTDILLLIVRLAVLMAVTLTVPVVIFPIRSSVTHLLCASKDFSWWRHSLITVSILAFTNLLVIFVPTIRDIFGFIGASAASMLIFILPSAFYIKLVKKEPMKSVQKIGALFFLLSGVLVMTGSMALIVLDWVHNAPGGGH

SEQ ID NO: 13 - DNA, 4563 bp, Homo sapiens
atctccagccggcggctgctccagggaggctgggcgcgatcctctccgcccgcggctccaacccgcactctgcgcctctcctcgcctttctcgcacctgctcctgcgccaggcccggagacccccggggcggcttcccagaacctgcggagcacaactggccgaccgacccattcattgggaaccccgtcttttgccagagcccacgtcccctgccacctctagctcggagcggcgtgtagcgccatggagaagagcaacgagaccaacggctaccttgacagcgctcaggcggggcctgcggccgggcccggagctccggggaccgcggcgggacgcgcacggcgttgcgcgggcttcctgcggcgccaagcgctggtgctgctcaccgtgtccggggtgctggcgggcgcgggcctgggcgcggcgttgcgcgggctcagcctgagccgcacgcaggtcacctacctggccttccccggcgagatgctgctccgcatgctgcgcatgatcatcctgccgctggtggtctgcagcctggtgtcgggcgccgcctcgctcgatgccagctgcctcgggcgtctgggcggcatcgctgtcgcctactttggcctcaccacactgagtgcctcggcgctcgccgtggccttggcgttcatcatcaagccaggatccggtgcgcagacccttcagtccagcgacctggggctggaggactcggggcctcctcctgtccccaaagagacggtggactctttcctcgacctggccagaaacctgtttccctccaatcttgtggttgcagctttccgtacgtatgcaaccgattataaagtcgtgacccagaacagcagctctggaaatgtaacccatgaaaagatccccataggcactgagatagaagggatgaacattttaggattggtcctgtttgctctggtgttaggagtggccttaaagaaactaggctccgaaggagaagacctcatccgtttcttcaattccctcaacgaggcgacgatggtgctggtgtcctggattatgtggtacgtacctgtgggcatcatgttccttgttggaagcaagatcgtggaaatgaaagacatcatcgtgctggtgaccagcctggggaaatacatcttcgcatctatattgggccatgttattcatggaggaattgttctgccacttatttattttgttttcacacgaaaaaacccattcagattcctcctgggcctcctcgccccatttgcgacagcatttgctacctgctccagctcagcgacccttccctctatgatgaagtgcattgaagagaacaatggtgtggacaagaggatcagcaggtttattctccccatcggggccaccgtgaacatggacggagcagccatcttccagtgtgtggccgcggtgttcattgcgcaactcaacaacgtagagctcaacgcaggacagattttcaccattctagtgactgccacagcgtccagtgttggagcagcaggcgtgccagctggaggggtcctcaccattgccattatcctggaggccattgggctgcctactcatgacctgcctctgatcctggctgtggactggattgtggaccggaccaccacggtggtgaatgtggaaggggatgccctgggtgcaggcattctccaccacctgaatcagaaggcaacaaagaaaggcgagcaggaacttgctgaggtgaaagtggaagccatccccaactgcaagtctgaggaggagacatcgcccctggtgacacaccagaaccccgctggccccgtggccagtgccccagaactggaatccaaggagtcggttctgtgatggggctgggctttgggcttgcctgccagcagtgatgtcccaccctgttcacccagccgccagtcatggacacagggcactgcccttgccaacttttaccctcccaagcaatgctttggcccagtcgctggcctgaggcttacctctcggcactggcattgggctccccagccggaactggttaccaaggacaaggacactctgacattcggcttgatccatgtccaggtgcaactgtgtgtacaccagggatctgtttggaaacaaccccttgagctgccaggctcaagaaatcatggactcacagggtcctgtgtggttacatcttggaaaaaatgcagatgtatttcactctccccggtcagctctgcatcaggtgttttctgagcaaaccaagggggtttatagtcatctgtcgcattgcctcgagttgcagtaattgaaaaaatgctcaaattcttagccatggctggcctttgctgagctgggactcaggtgtttaaagagtttgtgctatagctaggtgtggatagcttctgatccctgggttctgggagactgcaggtgccgcacattgtcaagttagaaatactccaggtgggtgttagcactgtggtggtctctggtccacagccttaggtaaacaacttagattctgaggtcaaagaaaaaaggagagggaatgcagccttgtgggggagaagcggggcagagggttctctaatctaatcaggacaggacaggtttcacatacaattgtcccagttcgcatcccagccctggggcacttttctgcttccttccagaggcctgggcctctgataacactttggctttttctccattcacgctgatttggcaaaaggccagagatgggcctccttccctggggaggtgtgatgtagttatcacattcaggacccttgttgatttatcatctattatttgaattcaactggacactctgtaaaatgctgcactgcagcaaaaacaaaaccaccaccaccccagagaaaaccatgtactaattggagtggggtacccccattcacaggttcccaggtcccctggctttggctgatttcaaaatatagagccctttcttgccagtacatccaagtttaaaattatcagcgaaatggtccatgtttttccaattacctgctgacacggttctaagctaagtgaaggggaagatctgagagcgtgctgtttgtggctgttgatgcatattcgtgatgtaacaggtcctggggcctcactttaccccatttgtaaaatggggctaatgtcacctgcctcttacctacctcagagggatttggtgaagcaaactgttaatcttcgaaaacgaccatttcacttcttggatatcaagtgctaacccagtatgttcttcttttttatgtaagggacagctttctccacagagtcctttctgctggtgaggacagcatttctgagcagggctttgttctctatgtgcattaggacttttatcatgcccttgttctgtgtgtagttacttgacagcatcaaatgccgcctcttcctaatgtccttcaagttttcatgaactagcaaccccaccttccaccatggttctgggcgcctgattttgctgtgactcccagacccagccactgtttctgccaccctgtaacaggccattaaagctccccagtgttcagcctccttcactcccttgttttccctgttgctatgtgtcacctgggccctacagacaggggcacacgcttatggatgtgtgtaccattgagatgagaatgggtagatggaacggagaccatcaagccacacccccttcttaaaactggggacatgagcctgagcagaaagggtgaagaagagccatgggacacagagttgacccagccagggggaaagcccagctctctttaaaccagctaagccattccagtctcctgtgaagccaaaagggaccaggaaccgtgcaaaggaaactggaaacttttccccgctgggtagagcatgttgctgatactcttctgttttcaagggaaacaatcacattgtttgattccaaatggtaaatgaacactcactattcttcaggcttcagtaaatctttttttcttccttcatatatatatacacaacacacacacacatatgtatatctatacacacatgtgtgttgtgtatatgcatgtgtgtgtgtgcgtgtgtgtatagttttagctccaagccaagcaagtttgtgtttggatagaggggaacttaactattaactacaagttgtatgtctgtggtatcttgattttcccatttctaaagatgaatttcacaaagccataaagcgtgaaattagagctggacttaagactcattggccgaccatcctgtgtcctggcctggccctgcagtaagaagcgtgtctgggtctggagaagggtgcttccgagagtgtgcaggtggcccttccccttggaggcgagaagagagaatgtgctgtctatcttcctggttttcagtccacagagtcggtagaccaggggttacgtgactggggaaaatctcacatctccttgtctgaaaacatttcccctgctgttctctttctaacatgttgtggtaaatctgttcagatactgctcatctgactgttttgtacatgtgacaattgccttaaaacctagcacagtcctcagaaatgaataccgtgtttccactggaa

SEQ ID NO: 14 - protein, 532 aa, Homo sapiens
MEKSNETNGYLDSAQAGPAAGPGAPGTAAGRARRCAGFLRRQALVLLTVSGVLAGAGLGAALRGLSLSRTQVTYLAFPGEMLLRMLRMIILPLVVCSLVSGAASLDASCLGRLGGIAVAYFGLTTLSASALAVALAFIIKPGSGAQTLQSSDLGLEDSGPPPVPKETVDSFLDLARNLFPSNLVVAAFRTYATDYKVVTQNSSSGNVTHEKIPIGTEIEGMNILGLVLFALVLGVALKKLGSEGEDLIRFFNSLNEATMVLVSWIMWYVPVGIMFLVGSKIVEMKDIIVLVTSLGKYIFASILGHVIHGGIVLPLIYFVFTRKNPFRFLLGLLAPFATAFATCSSSATLPSMMKCIEENNGVDKRISRFILPIGATVNMDGAAIFQCVAAVFIAQLNNVELNAGQIFTILVTATASSVGAAGVPAGGVLTIAIILEAIGLPTHDLPLILAVDWIVDRTTTVVNVEGDALGAGILHHLNQKATKKGEQELAEVKVEAIPNCKSEEETSPLVTHQNPAGPVASAPELESKESVL

SEQ ID NO: 15 - DNA, 2882 bp, Homo sapiens
agagccacgcggcacgcccgggaggctttctctggctggtaaccgctactcccggacaccagaccaccgccttccgtacacaggggcccgcatcccaccctcccggacctaagagcctgggtcccctgtttccggaggtccgcttcccggcccccagattctggcatcccagccctcagtgtccaagacccaggcagcccgggtccccgcctcccggatccaggcgtccgggatctgcgccaccagaacctagcctcctgcagacctccgccatctgggggcactcaacctcctggagccaagggccccacgtcccacccagagaaactctcgtattcccagctcctagggccaaggaacccgggcgctccgaactcccagctttcggacatctggcacacggggcagagcagagaagcctcagcgcccagcctggggaatttaaacactccagcttccaagagccaaggaacttcagtgctgtgaactcacaactctaaggagccctccaaagttccagtctccaggtgctgttactcaactcagtcctaggaacgtcgggtcctgggaaggagcccaagcgctcccagccagcttccaggcgctaagaaaccccggtgcttcccatcatggtggccgatcctcctcgagactccaaggggctcgcagcggcggagcccaccgccaacgggggcctggcgctggcctccatcgaggaccaaggcgcggcagcaggcggctactgcggttcccgggaccaggtgcgccgctgccttcgagccaacctgcttgtgctgctgacagtggtggccgtggtggccggcgtggcgctgggactgggggtgtcgggggccgggggtgcgctggcgttgggcccggagcgcttgagcgccttcgtcttcccgggcgagctgctgctgcgtctgctgcggatgatcatcttgccgctggtggtgtgcagcttgatcggcggcgccgccagcctggaccccggcgcgctcggccgtctgggcgcctgggcgctgctctttttcctggtcaccacgctgctggcgtcggcgctcggagtgggcttggcgctggctctgcagccgggcgccgcctccgccgccatcaacgcctccgtgggagccgcgggcagtgccgaaaatgcccccagcaaggaggtgctcgattcgttcctggatcttgcgagaaatatcttcccttccaacctggtgtcagcagcctttcgctcatactctaccacctatgaagagaggaatatcaccggaaccagggtgaaggtgcccgtggggcaggaggtggaggggatgaacatcctgggcttggtagtgtttgccatcgtctttggtgtggcgctgcggaagctggggcctgaaggggagctgcttatccgcttcttcaactccttcaatgaggccaccatggttctggtctcctggatcatgtggtacgcccctgtgggcatcatgttcctggtggctggcaagatcgtggagatggaggatgtgggtttactctttgcccgccttggcaagtacattctgtgctgcctgctgggtcacgccatccatgggctcctggtactgcccctcatctacttcctcttcacccgcaaaaacccctaccgcttcctgtggggcatcgtgacgccgctggccactgcctttgggacctcttccagttccgccacgctgccgctgatgatgaagtgcgtggaggagaataatggcgtggccaagcacatcagccgtttcatcctgcccatcggcgccaccgtcaacatggacggtgccgcgctcttccagtgcgtggccgcagtgttcattgcacagctcagccagcagtccttggacttcgtaaagatcatcaccatcctggtcacggccacagcgtccagcgtgggggcagcgggcatccctgctggaggtgtcctcactctggccatcatcctcgaagcagtcaacctcccggtcgaccatatctccttgatcctggctgtggactggctagtcgaccggtcctgtaccgtcctcaatgtagaaggtgacgctctgggggcaggactcctccaaaattacgtggaccgtacggagtcgagaagcacagagcctgagttgatacaagtgaagagtgagctgcccctggatccgctgccagtccccactgaggaaggaaaccccctcctcaaacactatcgggggcccgcaggggatgccacggtcgcctctgagaaggaatcagtcatgtaaaccccgggagggaccttccctgccctgctgggggtgctctttggacactggattatgaggaatggataaatggatgagctagggctctgggggtctgcctgcacactctggggagccaggggccccagcaccctccaggacaggagatctgggatgcctggctgctggagtacatgtgttcacaagggttactcctcaaaacccccagttctcactcatgtccccaactcaaggctagaaaacagcaagatggagaaataatgttctgctgcgtccccaccgtgacctgcctggcctcccctgtctcagggagcaggtcacaggtcaccatggggaattctagcccccactggggggatgttacaacaccatgctggttattttggcggctgtagttgtggggggatgtgtgtgtgcacgtgtgtgtgtgtgtgtgtgtgtgtgtgtgtgtgtgtgttctgtgacctcctgtccccatggtacgtcccaccctgtccccagatcccctattccctccacaataacagaaacactcccagggactctggggagaggctgaggacaaatacctgctgtcactccagaggacattttttttagcaataaaattgagtgtcaactatttaa

SEQ ID NO: 16 - protein, 541 aa, Homo sapiens
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 17 - DNA, 1946 bp, Homo sapiens
agtcccgggcccggagccagcgcatgcgcccgcctgtgggcgctgtcccggctgcgagggccgtgagctcacggaccgacggaccgacgggcggccggccggacagacggggcagcgcagggagcggggacgcggcgggacagcgacatggccggccacacgcagcagccgagcgggcgcgggaaccccaggcctgcgccctcgccctccccagtcccagggaccgtccccggcgcctcggagcgggtggcgctcaagaaggagatcgggctgctgagcgcctgcaccatcatcatcgggaacatcatcggctcgggcatcttcatctcgcccaagggggtcctggagcactcaggctccgtgggtctggccctgttcgtctgggtcctgggtgggggcgtgacggctctgggctccctctgctatgcagagctgggagtcgccatccccaagtctggcggggactacgcctacgtcacagagatcttcgggggcctggctggctttctgctgctctggagcgccgtcctcatcatgtaccccaccagccttgctgtcatctccatgaccttctccaactacgtgctgcagcccgtgttccccaactgcatcccccccaccacagcctcccgggtgctgtccatggcctgcctgatgctcctgacatgggtgaacagctccagtgtgcgctgggccacgcgcatccaggacatgttcacaggcgggaagctgctggccttgtccctcatcatcggcgtgggccttctccagatcttccaaggacacttcgaggagctgaggcccagcaatgcctttgctttctggatgacgccctccgtgggacacctggccctggccttcctccagggctccttcgccttcagtggctggaacttcctcaactatgtcaccgaggagatggttgacgcccgaaagaacctacctcgcgccatcttcatctccatcccactggtgaccttcgtgtacacgttcaccaacattgcctacttcacggccatgtccccccaggagctgctctcctccaatgcggtggctgtgaccttcggggagaagctgctgggctacttttcttgggtcatgcctgtctccgtggctctgtcaaccttcggagggatcaatggttacctgttcacctactccaggctgtgcttctctggagcccgcgaggggcacctgcccagcctgctggccatgatccacgtcagacactgcacccccatccccgccctcctcgtctgttgcggggccacagccgtcatcatgctcgtgggcgacacgtacacgctcatcaactatgtgtccttcatcaactacctctgctacggcgtcaccatcctgggcctgctgctgctgcgctggaggcggcctgcactccacaggcccatcaaggtgaaccttctcatccccgtggcgtacttggtcttctgggccttcctgctggtcttcagcttcatctcagagcctatggtctgtggggtcggcgtcatcatcatccttacgggggtgcccattttctttctgggagtgttctggagaagcaaaccaaagtgtgtgcacagactcacagagtccatgacacactggggccaggagctgtgtttcgtggtctacccccaggacgcccccgaagaggaggagaatggcccctgcccaccctccctgctgcctgccacagacaagccctcgaagccacaatgagatttttgtagagactgaagcagttgtttctgtttacatgttgtttattgaggaggtgttttggcaaaaaagttttgttttgtttttttctggaaaaaaaagaaaaaagatacgactctcagaagcctgtttaaggaagccctaaaatgtggactgggtttcctgtcttagcactgccctgctagctcttcctgaaaaggcctataaataaacagggctggctgttc

SEQ ID NO: 18 - protein, 523 aa, Homo sapiens
MAGHTQQPSGRGNPRPAPSPSPVPGTVPGASERVALKKEIGLLSACTIIIGNIIGSGIFISPKGVLEHSGSVGLALFVWVLGGGVTALGSLCYAELGVAIPKSGGDYAYVTEIFGGLAGFLLLWSAVLIMYPTSLAVISMTFSNYVLQPVFPNCIPPTTASRVLSMACLMLLTWVNSSSVRWATRIQDMFTGGKLLALSLIIGVGLLQIFQGHFEELRPSNAFAFWMTPSVGHLALAFLQGSFAFSGWNFLNYVTEEMVDARKNLPRAIFISIPLVTFVYTFTNIAYFTAMSPQELLSSNAVAVTFGEKLLGYFSWVMPVSVALSTFGGINGYLFTYSRLCFSGAREGHLPSLLAMIHVRHCTPIPALLVCCGATAVIMLVGDTYTLINYVSFINYLCYGVTILGLLLLRWRRPALHRPIKVNLLIPVAYLVFWAFLLVFSFISEPMVCGVGVIIILTGVPIFFLGVFWRSKPKCVHRLTESMTHWGQELCFVVYPQDAPEEEENGPCPPSLLPATDKPSKPQ

SEQ ID NO: 19 - DNA, 5772 bp, Homo sapiens
gaggtccggaagcggctgccgggcagcaaaggaggatggcgaggggctgatactgaacccgggaagggtgggctgtgctgaagccagagccggagccggagctggggccagaacccgagcagtgagttcctccactggctgccggctggcggcgctcgccgccttgggcaggacccacctcgccttcctcccggcgtggcagatgctccagctgccatgtccacgcagagacttcggaatgaagactaccacgactacagctccacggacgtgagccctgaggagagcccgtcggaaggcctcaacaacctctcctccccgggctcctaccagcgctttggtcaaagcaatagcacaacatggttccagaccttgatccacctgttaaaaggcaacattggcacaggactcctgggactccctctggcggtgaaaaatgcaggcatcgtgatgggtcccatcagcctgctgatcataggcatcgtggccgtgcactgcatgggtatcctggtgaaatgtgctcaccacttctgccgcaggctgaataaatcctttgtggattatggtgatactgtgatgtatggactagaatccagcccctgctcctggctccggaaccacgcacactggggaagacgtgttgtggacttcttcctgattgtcacccagctgggattctgctgtgtctattttgtgtttctggctgacaactttaaacaggtgatagaagcggccaatgggaccaccaataactgccacaacaatgagacggtgattctgacgcctaccatggactcgcgactctacatgctctccttcctgcccttcctggtgctgctggttttcatcaggaacctccgagccctgtccatcttctccctgttggccaacatcaccatgctggtcagcttggtcatgatctaccagttcattgttcagaggatcccagaccccagccacctccccttggtggccccttggaagacctaccctctcttctttggcacagcgattttttcatttgaaggcattggaatggttctgcccctggaaaacaaaatgaaggatcctcggaagttcccactcatcctgtacctgggcatggtcatcgtcaccatcctctacatcagcctggggtgtctggggtacctgcaatttggagctaatatccaaggcagcataaccctcaacctgcccaactgctggttgtaccagtcagttaagctgctgtactccatcgggatctttttcacctacgcactccagttctacgtcccggctgagatcatcatccccttctttgtgtcccgagcgcccgagcactgtgagttagtggtggacctgtttgtgcgcacagtgctggtctgcctgacatgcatcttggccatcctcatcccccgcctggacctggtcatctccctggtgggctccgtgagcagcagcgccctggccctcatcatcccaccgctcctggaggtcaccaccttctactcagagggcatgagccccctcaccatctttaaggacgccctgatcagcatcctgggcttcgtgggctttgtggtggggacctatgaggctctctatgagctgatccagccaagcaatgctcccatcttcatcaattccacctgtgccttcatatagggatctgggttcgtctctgcagctgcctacccctgccccatgtgtcccccgttacctgtcctcagagcctcaggtatggtccaggctctgaggaaagtcagggttgctgtgtgggaacccctctgcctggcacctggataccctgggccaggtaacctgagggcaggggagaggtggggtggcagacacgcagaagtgctactagtgacagggctgccatcgctcacctgtacctatttacacccagaactttccagctccccctcatcatgcctcctccttcctacctgcctcccctctgctggtgcacctcgcccaactcattcttactgcacagttcactttatttaacaattttcatgtcccccacctcatgttttcaccttttctgggccaggcatagattaagtaactgggaacgccccctctttataaagctgggcttctttctcatctctctcccaaatgttgtatctcagtattcttcctattcgagtctccagggggtggctggacctacctggtcatttgaaacaggcccccaagctggagtttttaatctggactctctggcttgctgtgacccctaaggcaatgcttctcttccctggattccttagtgtgggtcacagtactgtgttcttagttgctttagctcttaaaacatacgaagtgttgcctaaactgaaaatatttatcttttatttaaaatcagatttttgtttttagactgtcttagatctggggctattacgaatcacttcttcttcagtaaactttgactcaacttctcctgctgaaaagaagctcgctccagatgtctgcatgggtcctcggcactcttggctgaggactcaaaggttttaatcaggatcgtctaaaaatgtacctcggtgaggaggcacagattttgcctcctgttgaccagcctggtttcataccgaaaagacattgaaggactgcagaaatgtatgggtgcaccgggccgagggaagggtggctgagtgagaggcgtataaaatggggctgtgtgcatgcaggcccatgtttcagcctcagcccacgccaggtgaaaggatcagcaatgctctgttgccatcgtgctgggacgacaccagctctattgccaccgatgagtagctgaggtcagtgtgcacagagtttgaaattaagttaatagactttacagcagctggtctgacactacgcgcagtgctcggttgtttacaatcagtggggaaaagggcagaaccagtgcccggccccacactgcctctgtggcctggactttgaaaggaacccactgaacactaattatgagccctgtctttcccccagaatgcctccctgggtttcacaaacagccttgaggttggccctcctcaaggtcagccttcagatttgggagcaaacttcagagaaggcagaggaagatacattgccttgctgtgggctgcctcttctttcctcttggtgtgcgaagtatttcagaaggccattgatgaattccccctctttagctgtgtatttgtgcacgtgtgtgtgtacgtgcgtgtgtgtgtgtgttcctgtgtaagtaacagaccagactccttttctcttctgtcccgtcaccaggctcttgcttcactgcagatacagttcactctgaaagctggttgaaggagagcagcaaaaatgtatcaggggttttgcttctgtgtttcgccaaagctcataagggctgtgacccacccatatggccccagttttttctgtctcttctgttccaaagccaggagagctgacttccaggtgaagggatgggaaaagtggactctcattgtagtgactcccaacctacctaataatttgttaacttaggaatatgctatcattgttgacttgttcttccttaggagaaggacgattttcacccaccctttctgttctatggtggactcttaacaggtgctatgtgaccaggaatctagccgggagtagcagaggccctgtcttctgaagtctcaggcttagaagttaccaaagtgggctcagaaactgtcatctcctggttccaagttcgggctctggcagcccagccgctatcttagctgtctttcccagcggtgctaagagtggtctcagtgagaaggtagatgccaactggagggccagacctgtgtcctgcccatgtcctccttggtggacgtttctgtttactcagagctgctagagaccatcctgcccatccgagttctgagattgggactgtgatgttgggacctgaggactggatggtagaatactggggtcccccagctcttagcaggatgcaggctattgcttccacacccctggccgtgagaacgtggtatgtaggagagttggctgtagctttaggatttctggaagccaatttggcatggcctatttgatctctggcttgtgctcctgctacactgacagactgattgcgtggctctttcagaagacccaggaagggcagctccagccagaggaccttcctggctatgcaggtgcacagctcttaggcatcaagcaaagggtcagccagtcagtagtagtgggaggaagcccttcctcctcttatgcaagcagctcgcagccagcccagaatctcttatgcagcccaaagggcttcttgaggtagagagccctccccagtgtcttcccaggataagtagaaatatgatcacagagcaacggagcaaaggctttcgggagtgtgaggctgcatctgctggagcaaagggaaaccgtgggcttttccggccagatactcttgagctctgtgaccctgctcctgtcaccccaatttctccaagccagaggtagctttctcagagccccttggtggttctgtcctccaaatgctgctgttgggagcgggcttccagctctcagtgggcagtgcacctcctcctaaatgcaggcacttgctaggaagagtgtgagctggcgtcttctgttcaccctgcctagcagttgtcaccattcacaagtggcattatttatgttgtgctgctgtccagctgctaagatcccttcatctgcacaagccctggctagatatgtgtgaatgtgtggcatcatttcacttcagccgcccaattccatctctcctctgcagccagattcactggctgatgctcactgctcactgtctatccccacgaaatttagttttatcagtagtcttaaagtgttgatctcaaacaagtacattagaaaaatcatgtttcttctctctcatcttactttttcttctcagatttctcccttcctagaacattctctctgtttagcactaatgttcacctcgtattttttggaagtgcaaaaatctcaatttgtgtctgtttacagctctctctcctcactgctcacagcaaggggttctgtatcagtggatttcattttgtagctgctgagatgttaaggcaagcctcagcatctgcccctgctgggtgcacaatgctgcttcctcgaagagaagacacagagtccaagtggcaggacttgaggttggcttccactctgccttagaagttaattttccaaagtacattacaaatctctgaggccattaggggaaaaggaaggggtgtggtttgtctttgaaattacggttaatacttttagacagtaagtccggctggttgcagggctatttgccccgacagcatcagcctgtaacatttcttctctttcctttgtgccactgagtcgttccctggccagaggacataaatggtgctggtaggaggttatcagagtaaggaaggtagcagatataggtgcagggtgcctgtcattcactgtgttatttggtttaaatcaaagtgattctgggggaagctatgctctttcagtggataataaaattggtaactctattgtaaaacatgtcaatggtgtgtgaagaaaaatcaaccaatctgtaggtgttgataactagacagtactgtgtatgttacgtgcctgtgtggatgtgcacttccagcatggtatgtgtagcgatgtggatcatgccagagttcgtagatcctgttttggggtttgcacatggatcgtatgttaagctttttcttttcaataaatgaattttatttttatttttga

SEQ ID NO: 20 - protein, 476 aa, Homo sapiens
MSTQRLRNEDYHDYSSTDVSPEESPSEGLNNLSSPGSYQRFGQSNSTTWFQTLIHLLKGNIGTGLLGLPLAVKNAGIVMGPISLLIIGIVAVHCMGILVKCAHHFCRRLNKSFVDYGDTVMYGLESSPCSWLRNHAHWGRRVVDFFLIVTQLGFCCVYFVFLADNFKQVIEAANGTTNNCHNNETVILTPTMDSRLYMLSFLPFLVLLVFIRNLRALSIFSLLANITMLVSLVMIYQFIVQRIPDPSHLPLVAPWKTYPLFFGTAIFSFEGIGMVLPLENKMKDPRKFPLILYLGMVIVTILYISLGCLGYLQFGANIQGSITLNLPNCWLYQSVKLLYSIGIFFTYALQFYVPAEIIIPFFVSRAPEHCELVVDLFVRTVLVCLTCILAILIPRLDLVISLVGSVSSSALALIIPPLLEVTTFYSEGMSPLTIFKDALISILGFVGFVVGTYEALYELIQPSNAPIFINSTCAFI

SEQ ID NO: 21 - DNA, 4536 bp, Homo sapiens
ggctcactctggcaggtaggaacaggggagagtgcacctgctaccagtcaagctcagccagactgcaagaggaggcgaggcggagccagccgagggagtgaaccatggacaagttgaaatgcccgagtttcttcaagtgcagggagaaggagaaagtgtcggcttcatcagagaatttccatgttggtgaaaatgatgagaatcaggaccgtggtaactggtccaaaaaatcggattatcttctatctatgattggatacgcagtgggattaggaaatgtgtggagatttccatatctgacctacagcaatggtggaggcgccttcttgataccttatgcaattatgttagcattggctggtttacctttgttctttctggagtgttcactgggacaatttgctagcttaggtccagtttcagtttggaggattcttccattgtttcaaggtgtgggaattacaatggtcctgatctccatttttgtgacaatctattacaatgtcataattgcctatagtctttactacatgtttgcttcttttcaaagtgaactaccatggaaaaattgttcttcgtggtcagataaaaactgtagcagatcaccaatagtaactcactgtaatgtgagtacagtgaataaaggaatacaagagatcatccaaatgaataaaagctgggtagacatcaacaattttacctgcatcaacggcagtgaaatttatcagccagggcagcttcccagtgaacaatattggaataaagtggcgctccaacggtcaagtggaatgaatgagactggagtaattgtttggtatttagcactttgtcttcttctggcttggctcatagttggagcagcactatttaaaggaatcaaatcgtctggcaaggtggtatattttacagctcttttcccctatgtggtcctactcatcctgttagtacgaggtgcaactctggagggtgcttcaaaaggcatttcatactatattggagcccagtcaaattttacaaaacttaaggaagctgaggtatggaaagatgctgccactcagatattttactccctttcagtggcttggggtggcttagttgctctatcatcttacaataagttcaaaaacaactgcttctctgatgccattgtggtttgtttgacaaactgtctcactagcgtgtttgctggatttgctattttttctatattgggacacatggcccatatatctggaaaggaagtttctcaagttgtaaaatcaggttttgatttggcattcattgcctatccagaggctctagcccaactcccaggtggtccattttggtccatattattttttttcatgcttttaactttgggtctcgattctcagtttgcttcgattgaaacgatcacaacaacaattcaagatttatttcccaaagtgatgaagaaaatgagggttcccataactttgggctgctgcttggttttgtttctccttggtctcgtctgtgtgactcaggctggaatttactgggttcatctgattgaccacttctgtgctggatggggcattttaattgcagctatactggagctagttggaatcatctggatttatggagggaacagattcattgaggatacagaaatgatgattggagcaaagaggtggatattctggctatggtggagagcttgctggtttgtaattacgcctatccttttgattgcaatatttatctggtcattggtgcaatttcatagacctaattatggcgcaattccataccctgactggggagttgctttaggctggtgtatgattgttttctgcattatttggattccaattatggctatcataaaaataattcaggctaaaggaaacatctttcaacgccttataagttgctgcagaccagcttctaactggggtccatacctggaacaacatcgtggggaaagatataaagacatggtagatcctaaaaaagaggctgaccatgaaatacctactgttagtggcagcagaaaaccggaatgagatctcattgaaaaaaatatatgattgtataatgtgattttttttagaatagggggaaccttatttatttgtgtgttaactgaataggaaaatgtacatactatgttcatgatagtgtgatttttttcacatttaagcaggaatgcaatataaaaatgtgaatctcttaattctcagccatgtgcttattatatttctttttagattgtctatctgtataacacacacacacacacctaagagtctctatttcacaattatatttttgtaaatagtatatgcatttttaatacattggaggctttattttgaactaatttcttagagaatagttatattttctattacacaagtttaaaaatattattaacttgtattttcttaatatacaatctatcttttccacaaatatgagtgggaaataaatcagcacatttgaaagaaagtgttaaaactgaaggcctcacttaattagaaacgtgataaatatatggacaaatggactatacatactataagaggactgtagtttaatactttttacccaaatatgtttaaaaacttcgtgcatttgttacagctcatgttttctatatgaacttagtcattaatgttctttataaaaagtgaaataagatggaaaaataaggatcctacagccagtaagtgataaatctagaaaattgagttttgagtacctcttttcccatatacaatcttccttccttaggtaatttggaagaaaactatgacccatttaatttctattgtgtttcacaaaattaagtgttgttcattatactctctgaaatataggtttaatttcaaatagaatatggacttaaatgttaatgagaaaatggctttaatcaattctagcattttattactgtaatacagggctgatagagtgattttgtcttatatgagtaagttactacttacaggtgataacttgcatactattggaagataaacttgtcaaacttgtcaagaatgagaaaagccaaattagaaaatcctatgtcctagtttccttaccaaggataattaaatatatcactaagagctttatatattgattatatattgttgacaactggtttaagcatcatagcctatgatgataaacactgcctatatatgtaaatagcttttcatcaattcttaaatttcttaacctaggcttcagggagcatatgaaaccaaaattatatggaacattttctgtgtgtacatgtacatgcatttttctagggagagagtccgtaggtttatcagaatatcaaggaaaactgtgacccaaagaagtttaagaatcacatacactgctgctggctttttgtgcttggcaaatgagtgacaatagaagaaataatttttcttacacattttaaaacgttttctcttccttgtgattgaagatgaaaggagtaagaaattaaggcatttgtttaatttatactggtaacttatttaggggggaggggacatgaaggtaggtaaataggtaggcctctaattgaaccacctctctaagttatgtacgtatatataagctgaaattgtgtttgacattctgagggttttctttttctttttccttttttttttttttggtggggggctgggggtcagagtcttgttctgttgcccgggctggagtgcagtggcatgatctcagctcactgcaacctctgccttctggattcaagtgattctcctgcctcagcctcttgagtagctgggactacaggtgcccgccaccacaccagctaatttttgtatttttagtagaggcgaagtttccccatgttggccaggctggtcttgaactcccgacctcaagtgatctgtctacctcggcctcctaaagtgctgagattacaggtgtgagccaccgtgcccggcccattctaagggttttctttgaagacaggtcaaatgctgttagtaagtttcaggagattgttaattcctcagttataccagattttataaaatatttgagaatagatggctaacaagaggttagaaatacttttccttaattttaatccacagtatgttacatgcattctaccactacattttggtgctatttaaggtgtgcaattttctataggtgacttttgcaattcagggaagatttgggcatattaaatgaaagaatatctaattgggggaggtgtgaagggaaagaaattcttttcaaaagctgaccacaaagagtagttaaaagtttttgtcactatcttcacaagtgtgtaaagcacagatttcaacagagtgcttggcatattgtagggtgctcaatggtggtttttattattattactcagattccacagtggcaagaaacatcattctacataatggaaaacatttacatcaaatcccacttactttaatgcgaacttggagataatttatggtattgtattgtaaaccattaatgaaaactttttcacagttgagtgaaattaaaatcactatatctcaa

SEQ ID NO: 22 - protein, 642 aa, Homo sapiens
MDKLKCPSFFKCREKEKVSASSENFHVGENDENQDRGNWSKKSDYLLSMIGYAVGLGNVWRFPYLTYSNGGGAFLIPYAIMLALAGLPLFFLECSLGQFASLGPVSVWRILPLFQGVGITMVLISIFVTIYYNVIIAYSLYYMFASFQSELPWKNCSSWSDKNCSRSPIVTHCNVSTVNKGIQEIIQMNKSWVDINNFTCINGSEIYQPGQLPSEQYWNKVALQRSSGMNETGVIVWYLALCLLLAWLIVGAALFKGIKSSGKVVYFTALFPYVVLLILLVRGATLEGASKGISYYIGAQSNFTKLKEAEVWKDAATQIFYSLSVAWGGLVALSSYNKFKNNCFSDAIVVCLTNCLTSVFAGFAIFSILGHMAHISGKEVSQVVKSGFDLAFIAYPEALAQLPGGPFWSILFFFMLLTLGLDSQFASIETITTTIQDLFPKVMKKMRVPITLGCCLVLFLLGLVCVTQAGIYWVHLIDHFCAGWGILIAAILELVGIIWIYGGNRFIEDTEMMIGAKRWIFWLWWRACWFVITPILLIAIFIWSLVQFHRPNYGAIPYPDWGVALGWCMIVFCIIWIPIMAIIKIIQAKGNIFQRLISCCRPASNWGPYLEQHRGERYKDMVDPKKEADHEIPTVSGSRKPE

SEQ ID NO: 23 - DNA, 2301 bp, Homo sapiens
tccagtgagttgcaagcctaatgctcacctgcagagacagaattcctgagtgaacgaacagagcagctcctcttccatctccagggtctcattctgttgcccaggctggagtgcagtggtgcgatctcggctcacagcaacctctctgcctccagaaatgacctccaccttcaacccccgagaatgtaaactgtccaagcaagaagggcaaaactatggcttcttcctgcgaattgagaaggacaccgagggccacctggtccgggtggttgagaagtgtagcccagcagagaaggctggccttcaagatggagacagagttcttaggatcaatggtgtctttgtggacaaagaagaacatatgcaggttgtggatctggtcagaaagagtgggaattcagtgactttactagttctggatggggattcctatgagaaagcagtgaaaacacgggtggacttgaaagagttgggtcaaagtcagaaggagcaaggtttgagtgataatatactttcccctgtgatgaatggaggtgtgcaaacttggacccagccccggctctgctatctcgtgaaggaaggaggcagctatggcttctctctgaaaactgtccaaggtaaaaagggggtgtacatgactgatattacacctcaaggtgtggctatgagagctggagttctggctgatgatcacttgattgaagtgaatggagagaatgtagaggatgccagccatgaggaagtggttgaaaaggtgaagaagtcaggaagccgtgtcatgttcctgctggtggacaaagaaactgacaagcgtcatgttgagcagaagatacaattcaaaagagaaacagccagtttgaaactgttaccccaccagccccgaattgtggagatgaagaaaggaagcaatggctatggtttctatctgagggcaggctcagaacagaaaggtcaaatcatcaaggacatagattctggaagtccagcagaggaggctggcttgaagaacaatgatctggtagttgctgtcaacggcgagtctgtggaaaccctggatcatgacagtgtggtagaaatgattagaaagggtggagatcagacttcactgttggtggtagacaaagagacggacaacatgtacagactggctcatttttctccatttctctactatcaaagtcaagaactgcccaatggctctgtcaaggaggctccagctcctactcccacttctctggaagtctcaagtccaccagatactacagaggaagtagatcataagcctaaactctgcaggctggctaaaggtgaaaatggctatggctttcacttaaatgcgattcggggtctgccaggctcattcatcaaagaggtacagaagggcggtcctgctgacttggctgggctagaggatgaggatgtcatcattgaagtgaatggggtgaatgtgctagatgaaccctatgagaaggtggtggatagaatccagagcagtgggaagaatgtcacacttctagtctgtggaaagaaggcctatgattatttccaagctaagaaaatccctattgtttcctccctggctgatccacttgacacccctccagattctaaagaaggaatagtggtggagtcaaaccatgactcgcacatggcaaaagaacgggcccacagtacagcctcacattcttcttccaattctgaagatacagagatgtgatgaaaacaagtaatagctttggctgtttatttgatagctgtttctgggtatttaataggaatcctttctcaaggaatgagttgtgacctgtttactgtctctttagaagaaaaactccactggaaaccattcaccatgtgtgattgtcttctgttatcatttgtcttacaggcggctattgcagacggctaatttatgcttaacttaggaagagataaggcaagagctagatttttttcatgtgatcttttccaagcttcaacttaacttaactacatttctctgtatgatgatgtctcttacttctacaggttccttgagcaccaaagatgattcataactctgtataggtgacagctgcttataaaagcatcttagcagataagcctattaaaattgtgcttttgtaacaatgttgtggttgctagaataaataccattaacaaatgccttttgagtatgcttgatagtgcttttgttttggattcactttttatgctttaaccttcatttgcctctagaaacccaaaacacaataaagtacagaataagaccttagtaataaaattcagaattttcttaaa

SEQ ID NO: 24 - protein, 519 aa, Homo sapiens
MTSTFNPRECKLSKQEGQNYGFFLRIEKDTEGHLVRVVEKCSPAEKAGLQDGDRVLRINGVFVDKEEHMQVVDLVRKSGNSVTLLVLDGDSYEKAVKTRVDLKELGQSQKEQGLSDNILSPVMNGGVQTWTQPRLCYLVKEGGSYGFSLKTVQGKKGVYMTDITPQGVAMRAGVLADDHLIEVNGENVEDASHEEVVEKVKKSGSRVMFLLVDKETDKRHVEQKIQFKRETASLKLLPHQPRIVEMKKGSNGYGFYLRAGSEQKGQIIKDIDSGSPAEEAGLKNNDLVVAVNGESVETLDHDSVVEMIRKGGDQTSLLVVDKETDNMYRLAHFSPFLYYQSQELPNGSVKEAPAPTPTSLEVSSPPDTTEEVDHKPKLCRLAKGENGYGFHLNAIRGLPGSFIKEVQKGGPADLAGLEDEDVIIEVNGVNVLDEPYEKVVDRIQSSGKNVTLLVCGKKAYDYFQAKKIPIVSSLADPLDTPPDSKEGIVVESNHDSHMAKERAHSTASHSSSNSEDTEM

SEQ ID NO: 25 - DNA, 3983 bp, Homo sapiens
gtgtagattcgctggaagcagctggaggctccagttctcatctgctcaggtgtccccggcgccttggcgaactcggccactccagttcctcacgtggtgagcactcagggcagcgggtcgattttccgaggtcccatacctgggtttgaggggcgcggctcgcagcggcgggtgcaggggcgactgccagccctcaccccgcctcggggtgcgttcggaggccgacacctggaggacgcctccagtccccgcgggacgccacgcctgcgcgccagggatccgggataagaagtgcgcgccgggctccggctgcgcgccgcggggccaccagtttgcgcgcagggctcaggcgaccgtgcggccatggacacgccacggggcatcggcaccttcgtggtgtgggactacgtggtgttcgcgggcatgctggtcatctcggccgccatcggcatctactacgccttcgctgggggcggccagcagacctccaaggacttcctgatgggcggccgcagaatgaccgcagtgcccgtggcgctgtccctcaccgctagcttcatgtcagccgtcactgtcctgggcaccccctccgaggtctaccgttttggggccatttttagcatctttgccttcacctacttctttgtggtggtcatcagcgcggaggtcttcctcccggtgttctacaaactgggaattaccagcacctacgagtatttagaacttcgatttaacaaatgtgttcgtctctgtggaacagtcctcttcattgttcaaacaattctgtatactggaattgttatttatgcccctgccctggctttgaatcaagtcacaggatttgatctgtggggcgcggtagtggcaacgggggtggtctgcacattctactgcacactgggtggtcttaaagcagttatctggacagatgtttttcaaattgggatcatggtggctggatttgcatccgtgattatacaggctgtggtgatgcaaggtggaatcagcactattttaaatgatgcctatgatggtggaagattaaatttctggaattttaatcctaaccctttgcaaagacacaccttctggacaattattataggagggaccttcacatggaccagcatctacggtgtcaaccaatcccaggtgcagagatatatttcttgtaaaagcagattccaggcaaaactgtctctctacatcaatcttgtgggactctgggcaatcctcacatgctcagtgttttgtgggctcgccctatattccaggtaccatgactgtgatccttggacagccaagaaagtgtctgcaccagaccagctcatgccttatttggtactggacattctgcaagattatccaggacttcctggactttttgtggcctgtgcttacagtgggacattaagcacagtgtcctccagtattaatgccttagcagcagtaactgtggaagatctaatcaaaccttacttcagatcgctctcagaaaggtctctgtcttggatttcccaaggaatgagtgtggtgtatggagccctgtgtattggaatggctgcgctggcgtcacttatgggagctttgttgcaggcagcactcagcgtatttggtatggttggtggaccacttatgggcctgttcgctttgggcattttggttccctttgccaactcaattggagcacttgttggtctgatggctggatttgccatttctctatgggttggaattggagctcaaatatatcctccacttcctgagagaacattgccattgcaccttgatatccaaggctgtaacagcacctacaatgagacaaatttgattacaaccacagaaatgccatttactactagtgtttttcaaatatacaatgttcaaaggactccactgatggataactggtattctttatcatatctgtacttcagcactgttggaactttggtaacattattagtggggatacttgtcagtttatcaacaggaggaagaaaacagaacttagaccccagatatatactaaccaaagaggactttttatccaattttgatatttttaagaaaaagaagcatgttttgagctataaatcacatccagtggaagatggtggaactgataatcctgctttcaaccacattgaattgaactcagatcagagtggcaagagcaatgggactcgtttgtgaagctgctctgatactagatatccttaaatgatgtttcaattttatatgttttctaagataattggatcaggttttctttgtgtgtgtgtgtgtgtgttgtatcatgagtgtttgggggataagtttttgttaaaacaaagtctggactatcttcatttactacatcattaattgatgttactctggagtttagaattctggcattgacatttccctctctttcctttatttcgatgaagctataattgtgaaaattgtaactacatagatgctgaaaggctaatacacacatatgcacatgtatttgattgtcaaaggtatattcttaaatttgggtattattgaaaatattttccatgccttggtgctagcatataagtttggaagtttgccaacatcacaattcatcttgaaaagagcttttttccctcctaccacatacaccattcttagggagcaatgaggtaacaggtctgtgttgtctagatctttgctttttatccccctatcagtccagggcatatactaacctgcaaactgattctgaatcaggaaggtggtaatcaataagtattctggctgggaaagaccgtgggcccaatgatcaaagtcttcttggtgctgttcattaattcttgtgccttttggcttgttttctagagtttctgggctttggctgctgatactgcctttcttagactgtaatttttatctgcatgcccagtttctgacctatcaacttgggttttattgtgcactctaactgagcttgtcttcataattttctgtttattgccctgggcttggatatgtctcaagacactcatgtgaatcatgccaccccaaatcctggcttatcaagtcccagactataaattatgaactcccattagcttggtactaacatatacttgatgtaggtatttatggacttgatgatccaagaatattatattcttcaaaatggttaagctccatggagttagatgactacacttaatgctattaagttgaacttttgaatgtcaactaatttgcaatcaattaaagatacatatgcctagaaattttgaaatttcggtatatttatccagttaaagggctaaattatataagcaaacactactttttttaaaacgtctggactcaaaaaatgctttgttccatgttttaaaatttttaagtagcagtctcaaagttgcttagctgtttattttgctatgttcctagctaagagtttggttataggagttcatcaataacttattttttgtacagttcccacattagatactgtttaaaagttcttttttaaactcaattttttttagaaacataagagaaatatttagatacatacaaatgtttttatgattaaataattttatgcttattttctgatacgtgttatttaggtaatcatgccctgtacatttagaggttgctaactgacaatgttaagaaatttaaaaaaaaaaaaagcctgggcatgatggctcatgcttgtaatctgaacatttgggaggctgaggcaggaagatcgcttgaggtccagagtttaagtccagcctggaaacatagttagacctcatctctacaaaaataaaaaataaaaataaaaaaaacttagctaggcatgttgccacatacttgtagtcccagttattagggaagctgaggtgggaggatagcttaagcccaggatttcaaggctgcattgagctatgattacaccactgcactccagcctgggtaacagagtgaaatcttgtctctgggaaaaaaaa

SEQ ID NO: 26 - DNA, 54734 bp, Homo sapiens
gtgtagattcgctggaagcagctggaggctccagttctcatctgctcaggtgtccccggcgccttggcgaactcggccactccagttcctcacgtggtgagcactcagggcagcgggtcgattttccgaggtcccatacctgggtttgaggggcgcggctcgcagcggcgggtgcaggggcgactgccagccctcaccccgcctcggggtgcgttcggaggccgacacctggaggacgcctccagtccccgcgggacgccacgcctgcgcgccagggatccgggataagaagtgcgcgccgggctccggctgcgcgccgcggggccaccagtttgcgcgcagggctcaggcgaccgtgcggccatggacacgccacggggcatcggcaccttcgtggtgtgggactacgtggtgttcgcgggcatgctggtcatctcggccgccatcggcatctactacgccttcgctgggggcggccagcagacctccaaggacttcctgatgggcggccgcagaatgaccgcagtgcccgtggcgctgtccctcaccgctagcttcatgtcagccgtcactgtcctgggcaccccctccgaggtctaccgttttggggccatttttagcatctttgccttcacctacttctttgtggtggtcatcagcgcggaggtcttcctcccggtgttctacaaactgggaattaccagcacctacgaggtaaggggcagggtgggctgggaccatgcagggcgcgggggaaggggactctgcagacctctggaggcgttttcttgggggcagactgtcactgccacatcgaaatctctccccgtccatcgtcatcatcaccctcccttcactcctccccgtcccccgatcttcacctggcatctttcccttctactgagaggcgtcctctaagggtgaaaaaattcttgggatttactctcctgggcttagtgaaaaaaagaggcttccaaagtgaacggattgcaacagtagtgctcgctatggtcctttctacctttagcatctttgattcccaggcaaggggaaagatttttggggaaggtaagttcttcaggtctcaggccctgcttcttgaaaggaatactcttgttccaggtcctcagccccattcactcttccaagatagttggtaaagaatttgtactccccttccttccccttccacacccaccccctttccactgaaacaggcacccaagtggctaaggtgtaccagtacctgtatttcggaccagattctaccacttactagctgggtgaccttgggtgacttacccaaccttttgtgcctattcctcaaaaataaaataatatcacctacctcatccagttgggaggattaaatgagatgatgcaaggtgtagcatttagaatagagtttgacacagagtaagtgccagataggtattggccataattactgtggtggtgctagttgtgatggtggtagttatcttgggatcagtaggaaaattaggcagcaaggttctcaaacagctctgccttctctatggaaagacaattaaggaaaactgtctttcctctattgcacaggtcccctggctataggttttctcaagttgtctgcaagaaagggctttgaaaagatgatgcacgtttagtttaaagagctgttgaatgggaggtgggggcgggatcaaagctcaggggagtggcggggcccagttcagtgccttcagtggtctccctcacccagcactggtccttgagctacatgggacaagcctgcagaagtttcctctctattaggccattacccagaggactccaatcctgagattctggcaggtggtttcctagtgaggctggttcaactggtgctggggacagggccttaacggcacaacaaacattggtgacacactctgcctgggctctagtgacatcttccagggcgaggcacccctgggcagcattgccttccattcactctggggctgtgtttgggagagctcaataattatcccagaagtgagcaggatgcagtttctcaggtttgctcagaagcgacaaactggcgaaattccccagaccaaattcacttctcagtcattttttttttttacttctccttatagtgagaagaaataaaatataatttcagactatgacttctttcgagaactcaaaaggctggtaacactgggcccatgctccctgtggccacaggaactcaagtttatagctattgctctcactctctattaaacttctatattgaagtcttatctttgtgttttcatggttactgtttttttggtagagaaatttttatctgcatttatgtctttatcaaaaaatgtgaaaggcaaaatggacaggggtaaagggatctcttgtttccagggagatgtaagaaaacatatatcttagtgggtgggaggaatattccaacatgttaatatgtgaacacctggccactgtcacttatgtatgttacctgatggaacctggaggcatctgagtctgtgagctgtgatctatagctaaccaattcccaaacttggaaagggtttgaagaatctggcaggaggacttcagtccccacacatgaatgctgctctcataaccactgacccatgcctcttcggggctacaacattaactcaagcagaaacagatttccctcatgcttgcagtggtagaggctaagttagcaggggttccagctaaaaatgtcaaaaagcaaaagttcattgaatgctgaacacatgccaggcactgggctgcattgcatgttttacatacattatctgactcacattcagaacaatccttatgaggcaagtactatcaagagccccattttgtggacagagactcagagtaagttaagctactaggaatgtgaagcagcgtagacatatgcctagcagcatactggctgcttctgaaggctctgatgagtctgttataaccccaagcaatgcttacgccagtgcagagagaacaagggaagcaatataggacacgtgaagttgtgggcacactgcaggcatccacctccagggtgccaggcagttgggaacggggaagttaggcaacatgctaagaccctcccaattggtcatggcaaagactttcctgtggagtatattgtttattgttttatgctgagaggtacagaatctatacagaatatgctgtacagtgttgagtgattgataccctggcattcttgctcaagtatgaaatctctccctgttgacatttctttagtgaaaattagctgttcacaagcattttttttcatccatcactgccttcaaaatgtagtcctgataggtgtttagcaacttccagacccaagtataatgcaggaatccaatttaatccaccaattcttcaagatccttctggagccaactaaatctgtgttttaaattgagttgcttagaccttatgtttctttgttatttatatatttattcatatcaacagaagcaattcaaagttctgaggaccacgagagatcttgattaatttaggcacctatcattactctgtaattatgtctagaagtcttacaagtattccctaaggctgctgcacagtgacttgggcatctggtccagtctccgggtatttcttcagcagcctagtaattgatggctgagatcgtaatagacttcttggttctgtcttgagttggtccttgggcttaaatcacaggtttccctggatcagttgattaacttacttgcttcagtttggttcaatttatttgggtctccagcaatttggattcatgtacctagaaaaaacaaagacttctcttctattgatgtccattcacatgtttagtgattcatagagatttacaaaacaaacaaaccaaaaataaacttctaaagaaatataataatactgccttgttcccagacttaataaacttctccagtcttctaaaaaggcttccaaaatatccctgaatgattttgacacttgtaatttattgagattcaattttctccaggcccattgatttggataccaataggttcttaatatcttactttttgagtttacatttccccatccatttatgtcactgaatagtctccaaaccctgcaaaatgagagggaaacaaattattattatgcccctcattttaaagagaaagaacctgacatttggagaatgattgccacaagctgatggagtcaggcgtgggactgggtcaagggctggaactcttcctccagtgtactacaaggcactgtgtatttttgtcaccgatcagtgatctgggagtgaggtcttactctgtgttagtttcacccattcacttattgagtcttaaatatgtagtcagattatttacttatatatgcagaaaaatgtgcacagattttggcatcaaaggttttaaatgtgtactgtggccctggcaagtttgcaaggttttcaacttgatgttctatctgaggcccagtgttcttgtctctaacatgggtttatacacagtacctggctcatagagttgttgggaggaccatgcacacagcaagtactctgtatgtgttagccatgatcacggggcaaaaaaatcagatgtcttttcctctgattattacctaatgccagtttttccttcctcttcttttctactctatgaagattgttgtcagccttaaacttcttagtaattatattcaagttaaagaaaatcttagctcccagctctgaaattctatgatcctaactctgtaatatgtatattataatacattatataacatagtagacaagcagactatgttataagacatagcagatttcaagtagaataggaaaaattggcatggtcattggcactgaacagaaactgacttgggtgatttaagtttaaaaaaacaattgttagaaggacatgggggtagctcatagaacagagggacaggctgagtaagcaagccttggaaaggacagaaaccggggcagccttttcaggccactctgtcaggatcagtcattttttgtctttgtatcctgcctttaagattcaaatacccaatggaaggaaaagctgattggctgagctgggatcatgtgatttcctcttggtcagaagaggcagggcaattggctgactattccatcacaatagtctgcagtcagaaagggttgatgccccaggtgagccaagaaaaatgtatggagtagataccaagagaaagagaacaacagattgctcgctctaaaaatacagaaagtaatttcatttatatgacattctggaaaaggccaaactagagagggaaagcagatcagtgattgctagggtttggcgggggagaagagggcttgatcatgaaggggaagcccgagggagttttctgggtgatggaactgtcttgtgtcctgattgtgacaggggttacatgaatcagtgtgtgttaaaacccattgaactgtactctaaaacaaaagagtcaattttgctgtgtataaataaaaataacactaaaataaaaatacagaatacaaatcagttatgaagttgctttacatttctaaatttaaattttcttcttgagctgctgattttaaaaaaggcatccagaggattcgcataatttttttttttgcaacagtatttagaacttcgatttaacaaatgtgttcgtctctgtggaacagtcctcttcattgttcaaacagtaagtagctctccatttattttatcttttgtcagctaaaatgtttttaaccatggcatctggattaagcttacctgggaatcacataaaagacaaaaaagagatgttgaaaaaatgagggaacaaaaaaggaaaaacacttgtcactgtacagcatcaacccttaagatcatcaaacagtgtttcagtaaatgctttacttcctgggatttaagtgaatgttaaatattatactgataaagcaacagtcggaaaatatgcttccctttatctgacctcccttcacctccaccaaggtgacaggaagggcattactacatgacaaagatatttgttgctgacagtgggaattctaacaaaaggaaaacaagtagcatgttcacagtatttctgtaacatattaataggtatgaaaaaattaacttccataggaagacagtaggaaataattttctttgaaatcgtactttaaaagtcagtgctcttttaaaaattagactacaagaaccaactaaagcagtccttagagtgtaaaacaacagaatcataaactctggaggactctttaaagccaaatactcaatccagtaattcaagaacatgcgcttatagatctactgattgacacaaagggaaagcaaggatttgccaagtggtcagtgacagagaatgctttccactgttcaccgtgcttctggaagattgtaatgatcattgtcatgactatttatatacacattttcctcttgtcagttaagcactttagggctgggtttgctaatggaggttgtggaagagatttgcattcttgtctctaattgcaattccacttctccaatcaaaagctacctaagggccagccgcggtggctcatgcctgtaatcccagcactttgggaggccaaggcaagtggatcacctgaggtcaggagttcaagaccagcctggccaacatggtgaaaccccatctctacaaaaatacaaaaattagccaggcatgagggcaggtgcctgtaatcccagctactggggaggctaaggtgggagaatcacttgaacccaggaggcagaagttgtagtgagccgagatcatgccattgcaccccagcctgggtgacagggcaagactctgtttcaaaaaaaaaaaggaaaaaaagctgccttaggatttgctgcagtgagacagagtgcctttgtaaattatgtaacttgactccattttatatctttgcaaattatataacttaaattttatcagtccttaacaactgcagtgtaaaaggaaggaatcctttggtgtctcttagagacttgagcctggtagcttgcattcaccaactgttcagaacctcattggatctttgttagagatgccaacagaaatcagaagtagggataagtgttaggaaggtggcctgtggtcatgtttttaaatcttcaacttggacagaataatgactgtggaaagttagttcatttttgcaaaaagaggggagctttaccacctcccatttgaaggacttcatagctctactcatgtaatataatcaaacattcaaaggtactgaatagatttttattttcataatatgcttttatagaataatcatggaatttgcttttatgggatatatttgaaagatcaagtgcaatcaaaattacattttgagaaaaagaccgtatttatcttactcactactgtaccccagatcaataaatagttaagtgtatgaatgaaaataatgaataatattaaaaagattgaatgtggttttcattggctttcaggatttttttagtgcaaatttatacttttgtttaatttatgaacagtaaaagtttagaaataggctttccaaatttttactattttcttgattaattatgcagggattaacagtttgaagacataattgaggggtcatcctcttttatattattattatttatttttttctaattatttcagatgtaggaaattttatttcaagcttgagttggtggatcagtgaccatttgcactaagcaccatataaaagtccgtatttttacataagccggtcacaaaaaaatatttgtaacttatgaccggtcataccgtaaacagaagagtcaactttacttaaatattttgcaagttacaaacaaattttattaggtgttttgaaactgttgttttaagtcataattgaagttataggaaaacaatcaatattttataactcagacgtaattcatgaattttataattcatataattcatttggctttctttctcccccctagattctgtatactggaattgttatttatgcccctgccctggctttgaatcaaggtacattttagagttgccagttaggtaactcacattttggggttcactttcaacaagccttattttctccttggggagatggggagatggaggaatgcttctagtaacctgcatcagctttacttagcggggcaggatgggttcagtgtctacactagcttctttgggtttttggatggacagctccaaagtgtctgtagaccacagaggtggacttctccaggtggtacttctctgggatgtgcccatcagcccattcactcctttagaattaaagctccctgtagccaaagtcaggattgacggcatcccctcttgtgaatctataacctggagcccatctccaggaagcttccctgtaattctgctcaggcctgttgtaaaatctggcagggagaaaagcttttcttctccacacttctaataaggcccaaaatgaaagagaaagagagccatgtgatttgaatgatcagactgctcctagtgacaaaaggacagatgtctgtagtgcccttacaaataaatttaggaagattgtgctgctcaacaaagtatctatactctctagatttgggaagataaatgcagtgaggctgggatagtttattgaagcaaacgttcatgctagtcatagtttcaaaaggctttggggaaacaccatgccctttgaattcttatctattcgaagtgaatttctctaaaacgtccttgtaaaatggacatgtggactctgtgatgggaagaatgtggtacaattcctggggttaaaatgacatgaagaaaacctactaattccacactctgttttcttgattttatgatagacatgacagtagttaccacctgtttctgaagtgaacaatattattaccaagaggaacttcatgtgtaaggtgctcttgaactctgaattctgggcatgttccacatcggtattaccaacatcacaagtggatcatctcatttgtcgagaactgagttataaacttcatgagttacctatttaggacttaagtgtaattgaacatattatggttttaaaatgcagttctgggaattaccagaggactgactttaatctgtgaagaaatcaagcattctgttcttatcaggctcaaaccactccctgagagtaaattagaatgaaagtgaaggtgttttgatgactagagtgtcagttgtgtgttttactgtgaccaaagctgtaggtacaggcatagacacagaacttagtgtgcaatagatgctcaaaaaagcttatttaactgaattgaaaattaacattctctaggtctaacctctttttttctttttaaaattttggaaatcctcatacaaatactaagagagaatagagccccttaatgtgctcattcatcactctatgttaatatttatcaactaatggtccattttaattcactcccccttacctctaatagattattttgctgcaaatatgttatgtcatttcatctgtatttcagtatgtcctttagaaataaagactttaaaaaccataatcataccatcattgtacctaaaatagtaatattaatttcttaatttcatatcaagtcagtgtttacatttccctgattttttttcagtgtttataagaatcaggatccaaataagcttatgtgattgcaatcagttgatgtctcttaagtttcccttttctgctttttaaaattgaaaaccagtttttctttctttcttttttcctgaccgcctgatgcctgttgagaaaactagtttttattgaggtatagttaacatacaataaaatgcacagagcatggggaagtgtacagttggatgagttttagtagttgcctaatagcttgtgtgactactaccccgctcaagatatagactatttccgtcatccactctcagaaattttccttgcatgtctttctaacaaatctcctatgggcccaagaaatcattttctgagttatgctaccataaactagttttccctgttgttagattttatataagtggaatcatatagagccttttcatgtctggtttcttttgctcagcaaaatgttttgagattcattcatgctgctacatatatatcagtagttcattccctttttttttttttttttttttttttttttttttttttttttttttgctggtagtacttcattttatgcctctaccaacatttttttatccattctcttgttatagacttgggttgtttccagttttaggctatagtgaagtaagggtgccaagaacattcttctaaaggttttgtgcattttgtttttctttttcacacctgttttcacttccattggaaatgaaatacatggatgtagtataaaattaaaaatataattctatttggacccagctgaggatagaaatgtgtgcaaatttacaagaaattgccaaacagtttttccaagtggctatatcgttttccatgtccactacgcaatatgtgagaattctagtcattccacatccttgccagcatttgatattattcttttttatttcagtcattctagtgggtattacctattattttattgttgtttcagttttcactttctatatgactattatggcttagcttattattagactattatggcttagcttattggcttctgaaataataaagtgaataaagacaaataacctaataaaaagtgagccaaagacttgaacacttcacaaaagcagatattcttctaatacattacatgatatatctattcatttacctaggcctttaaaaatttctcttagtgctcactttggcagcatgtatactaaaactggaacaatacagagaatattagcatggcccttgcacaaaaatgatatgcaaatttgtgaagcattccatattttttaaaaaaggaagaaaagaaataataaaataaaaagtctcttagtaatgttttgtagctttggatgttgaggtcttttactatttcttagatttattcctagataattgttgttttttatgctattgcaaatgtcattgttttatttttcaattattcgttgcaagtgtataaaaatacaactgacttttatatgttgatattccatcctgagaacttgctgaatttgctttttaaatcaagcagatttttggtgatcccttaggattttcatgttgtttatgaatagtgacaattttacttcttttctaatccttatagctcttagttctctttcctgtcttattgtaatgttaatgtggaaagttacattgactgaattttgaatattaaaccaatttttgcattcttgggataaaccctgcttgctcataatgtattatcctttttatatattaccgaattcgatttgctaacattttgataaggcttcataaatgaattgaaaagtgttccctcctccattttctgaaatagtttatataaaattgtgtatttatttcttacatgcttggtagaaatcaccctgaaggcatctgggcctactctttgtttttgtagaaagattttggtttatggattcagtttatttcgtaaatatagagttattcatattttaaaagtcttgttgggtcagttttggtaaattgtattttttagggaatgcttccatttggtctaacttatcaaattcattagcataaaactgcatataataccctcttgttatgcttttaatgtttatagtatctatagtgacatcccttctaattattcctaatatcgataagttttctatcttcttattttgatatatctttccagtattctattgatgttttaaatctcttctaagaatcagtgttttggctcattgattttctttgttttctatttcattaatttgtgctatttattgtttcttttcttctaataactttgggttttctagcttcttaaggtaggcgcttaggtcattgattttaaatatttcttatttcataaagcaaacatttaaagttattaatttttttgaaacactgctttatctgcagcccatatattttgataccttgtgtttttatttagttctcaaaatattttctaacttctcctgtgatttcttttgcccatgggttatttataagtgtattgcttaattttcaatatttgaggattttctatcatgtctttttgttatttattcctaaattaatactgctttggtcagagaactaatatgattttaatcatttgaaatttatggatttgttttatggcccaggatcaggtctgtcttgaacattccatatgctctcaaaaagaatgtatattgtgtgctttttggatgtaatgttctataaggtcaaaggtttagttggctaatagtgttatcacatctttgatatctttatggaattttttttacttgttctaccaattgctgagagaaggatgctaaaatctccagatataattatcgatttttctattgctgctatttctctgtcaatttttactgaatgtattgtgaggctcttttaaagtctctttttaaatttacaatctcccttctattccttttcatttaaattgcaacatatttgcagaagaaactgggttatttgttctgtccagttttccagattctaggtttaacaattacatctgtatggggtcatttaatgttcaccattcctctgtatttcttgtaaactggtagtagtttactagtagtagttagatctagactctttttcagactccacttcaactttttggaaatcatatttcataggcagtatctataagtttatttccttcaggagtcccagaatgtcatttgaaaaattatcagcagctattgatgatcattctagattcattatttcattgaggcttataaaatggagtattctagttctgtgaattcttcttcattagttagatggaatactgctataaagagaaacttcctcttatcaactatttgctttctctgaaaaatagtttgtataggaaagggaggataaatgtcagatttttccccttatgaattttaaaaataataaattggtttcctactatcccccaagggtgactgagatttttaaaaaattcttacgaactcatggatttaaaggtttgctgttatttcatagcattatcgttagtattattattgatgctcaaattgtcccatttttatccagccggagcccccttaagttcctcctgattttagccttggctgcatattggactcacttgcggggtgggggagctttcaaaaataccaatacctcagtccccatcaccagaactctgagttaattaggctgttgagtggcccagttatcaggattccataaatttcctatgttgattttaaggtgcagtcacaattgagagccatggatgtaatagttttagaatagcaactctaagccataatgaataatataatggcctaacacagttaaaaatattattctttgttgttcttttttcttgggtgtatctcagtaggaatgtatgttaaattactgtgttaaatattttgtgacatgtttcctctgtgtggtaatgtcacaaacttgatatacagttaggttccatttatttcattttgcaattgattttgaggagtttttttttctaatttaactttatatttatgtggaatatttatgtgttccaaagtgaaatctatacatcaaaatatgtttaaagtagcctgacttgtatctctgtcttctctaccctgttttctccctctcctaggagtaattttttggctttgatttatcctttaattttagtatatgtactgctgaaacaagcacaattcttttcttttaaaaaaatgaaataaagtccccacttcttagataaataggagcaaattataaagacttttctccatctagatcccattttggtagcacatatcttagtaacgccttcttcaaggactggtgaaattgcgtgtttacatggacacacagaggagaacaacacacagtggggcctattggagggtggaaggtgggaggagggagaggatcaggaaaaacaacaaatgggttaatgggtactaggcttgatacctgggtgatgaaataatctgtacaaggaacccccatgacacaagttcacctatgtaacaaacctccacatgtacatctgaacttaaaagttttttaaaaagcattaccggccaggcgcggtggctcacgcttgtaatcccagcactttgggaggctaaggcaggtggatcacgaagtcaggcgattgagaccatcctgtccaacatggtgaaacccgtctctactaaaagtacaaaaaattagctgggcatggtgctgcgtgcctgtagtcccagctactcgagaggctgagtcaggtgaattgcttgaacccaggaggtggagattgcagtgagctgagatcacaccactgcactccagcctggtaacaaagcaagacttcgccaaaaaaaaaaaaaaaaaaaaaggaattaccattacctttattaaatttctgaaatcagatccgcaatctgcaatgttataaagattacatttgaatcctttcgtgttgtcttcatttgaatctatacatcatagcaatttgatattgtcattatattgtcagtatgatacattatcctcaaattcaggtatttgcaacattacataaaaatagagtgtttatttgaaccgcgtctaatcgggagagagataagaaatacccatcacagtatcaaggagtttcttagcttaattacaatataaacgcctttccatgtattgaattaagccagtgtcagatctgtgtgtctgagagtagaaaattatcaaatacaattttaaactccatttgtttttgagacatttatgagatttggagctagttttagctttaggcaagtgggtatagaggaaggtgcctgataaatgatatagcttccttctctgatgatttttgaggattatttttattgtgcatatttcacaattatatatgcattctatttttttctacagtcacaggatttgatctgtggggcgcggtagtggcaacgggggtggtctgcacattctactgcacactggtacgtccaggacatatttcccttttcactctacccacttgctttgcaaaattgaaaattccagttgttgtataccgcaatcttgtttgtccacacttactctcctattcccaacctcctgcagtgtaactttttttgtgtgagaaataatctgtagtataatttgatcctttgtagaaaatggagcataactgaaattttttcttttatctgatccattgcaatggttcctaatcttgtctcttgattcatctgtactgttgccaagttattatatttgtgctcatactattcctcagcttaaaaacctatcataggtcataggcacagtggctcccacctgtaataccagcaatttgggaggccgaggtggaaggatcactggagcccaggaatttgagaccagcctgggcaacaaagtgagactctgtctctacaaaaaaacaaaaaaaattttttaaaattaggaggcatggtggcatacacctgtagtcccagctactcaggaggctgaggtgggaggatcgcctgagcccaggaatttgaggctgcagtgagctgtgattgcaccaccgcactcccgcttgggcagcaaagaaaaccctgtctcaaaacaaaaacaaatctgtgataacttccccttgcttataggtaggggtgacaaaatatgcaaaatcagtatggcatggcactgatattattatgtgtgctggtccgaggcagtgagaaaatcttatggaatccttgacactctatgtcttaaccctttaggtgctggatcttgtagaagatggggccctcagccaggtgctggttctgcttaggtcatagagaaacttgggaaatttcttgtggtggctagtacagatcacttatagtatataacagtggaaatgaccatctgagtgaaaaccaattaaatatcacctctgcctgtaggataaaattcatattatttactagtaatttgactcggatttttcttcccaacaatctcccgacaccactccactttattctgtggaatgtatctggccccattcagcatgccatgtagtttcactcctttatgtttttgcccatgctgttttctctgctgggcggaacctccatctttgcctctgcttatgtttgaagacttgatttaaatatatccttctgtatgaagattctcccaacctgactgtttcccttgagtgcacttcttagccaacattcatgtttatatcatttttctccagtatagtgtataagtgtgtagatgcttaataatgtctaatagaagcattaatcctaatatactttcccctcaaagggtggtcttaaagcagttatctggacagatgtttttcaaattgggatcatggtggctggatttgcatccgtgattatacaggctgtggtgatgcaaggtggaatcagcactattttaaatgatgcctatgatggtggaagattaaatttctggaagtaagtgtctagtacttgggtaactgaacacatcttttgtattctataaaaataatctctttattgaaatagtagatttacattaaaaaacaagccaacaaattgctaaggatgtggtagagcaaattgaagcagagaagtaaatacgtaaggagcctccctctgttctttaagggattaaacctgtcagatggtacttagctacatggtgcttagagcaatgtttccttctgagaagggacttaaagcaaaaaaagtattttcttccaggtattaaagtccagaataggttgaaaagtgggacagggtgataaggaaagagacagtggaaagttaagaaaaggcagcttctggccaggcacagtggctcacacctgtaatcccagcactttgggaggccaaggtgggtggatcacctgaggccaggagttcgagaccagcctggccaacatggcgaaaccccatctctactaaaaatacaaaaaattagccaggtgtggtggcaggcacctgtaatcccagttgcttgggaagctgaggcaggataattgcttgaacccaggaggcagaggttgcagtgagccgagatcacgccactgcacttcagcctgtgcaacagagtgagactctgtctcaaaaaaaaaaaaaaaagagaaaaggtagcctcttaagagacaaacactagcatattggattagcacctgccataaaaaaaaaaaagagacagagacagacactgaaagacagtgatttcattttgacaattctttgttttaagcaactttgagagtttctcttttgatattgccctggcaatctatagtatattaacatagtagcttatgattatgtattataattctatgtatgtgtatgtattatataatgaacattatctaatatgaagtattatttctagagaattgactaggaaagttacagtctatgcttcaaatgacagtacagcacatgatgtaacatgttttaagcagttattcagttttcctaaagaaagaacaatgaagagactagatatttcatcccaagtatatcacacaattcaaaagaatattgaaaatgccttccgttttggatcaatagtgttgtccttttgcaatatggaaagggacaacccatgttgtcttgaactagcctatcttctctttgagatcgcagccccgctttaacataggcagtttgaagaaaaaaaacccattttgcacttggtggctcttttctggtcttctgaaaataagcaccaaagtttgagaaaaagctttttcagaaattggacagggtcaatgtgttaatttacagggataaattttagtgaatcaactttgactattttcaatatttctttccttcttttagctcaagattcagattctaaggaaaagaggctctagttgcctagcttggaccttggttccaccccttggccagggcagaacaagatattttgactgatcgtcaattgagactatttaatggagaaatggtagtttccccaaagcaaacctgggttgcttttaccagaacagggagaggggaagtccaggaagtggaaacaaccaatgactactcttgattgctcccaaatctcttcctggtaggccaggctgaggagagagggtatggaaccaatcatttttctgcaagatagctgtcactttatatgaaggatacatatattggagcacagaagtgatagcttatacacattagtaagagatttttaaaaaaagattaaacatttttatgaccttagttttgaaagtcattaagtaacaataaaaagccttatttgtgtttcatacttttcaagagtatcccttgttattctgatatgttttgagaatatgatagacaaagtattgctcaagttaagataaatgaagagaagaaggatattgtattggtattttctgtgtcgtctgccattggcctatgtattctgtatggcctaaaccactaaagtgagtgttttaaatcttgtcatacttccctcagatagcttactgactccttttggctcttcagttagctaatatagtagcttctctctcttggggagaaagggtcaacagtcttgaaactcttacactttgatatgaaatgttagacatgaaatagaggctctcacatttccacaagaatgccagaatacacatacaattagagcacttaactgatctcaaatataagatttgaaatgaatttgcaaaatttcataattttaaggaagtctccatgaaagctaactttgcagaaagttttctagctcatattgtatatcagagatgaacaaaatcattccttccagtaaaaaagattaaaagatctttcaaacatggaagttgagctttccctgtgacaatgttttggacttatgtacatgatgtcataaagtggctttaaactattagtatttagcttgcacgcacagcattttaataaagcatgattacaatgacatgcagtttttagaaaatgcaaaacttgaaactgcattaattgactgatttgttgaacgtgaatatagattagaactatgattacatgtgctgggagaggaattctatacacaaaatgtgttgatttcgtgttcattttagtgccatctcgtctgctgtggtgcacttaggaatgtttttctctttctctttctctatgtttcatgttcttctctgattatatcatgtcagatttgtttaaatgacagtttccttagatcacaggagaacaagttatataaatgctcagaaggattgcaacattcatttcccccagtctcttctagtattttcagatctctgaaaatagtattttttatttcattgaacttaagttggaaatatttctaccttttaaattaattaataaatttttacatatttatgtatgtgttgtgtatataaatgtgcattgcttgcttccaaatatagtctaagagaattttttttagtctctgctaatagttcagatttgttttctgtttcttgtcatttagttttaatcctaaccctttgcaaagacacaccttctggacaattattataggagggaccttcacatggaccagcatctacggtgtcaaccaatcccaggtgcagagatatatttcttgtaaaagcagattccaggcaaaactgtaagtcacacaccatggtatatgaatcattaatagccatcagttgtctttatggaaactctttcataagtcacgtttagctcctttatgtcttttgtgtcatgaaattccaagagataaatgatatatttggttacaaaaggaccaagaacaaattgttactgtatgttttaaatcagctatttgaaaatatttataggaattattaatgaaaacaaatcagcatttattgtgcatctgtttgcataagaacccatgggggataaatatatatggcttttgtctgaagaaaattaacctcaaattaggaaaatacatgcaagtatgtgaaaaaataaaataatactggagatatgtaacagtttcatgaaatgatatagcaatagagaaaatgttccagagtacatatagtgctttatatttcattttcagacacagtcatggatttataaccctgctctaatatttgctatttgagtgatcctgggcacattctgcagtctctttgagctgcagtttctaaacttgtaaaaggagcataagaaatactatacacctcatgggaggtgtcgtaaaatactgtgtttggcatacgcatggtaaaggcctagtatatgtaagttcctttttccacttcaactgatgtgatgtgaaggtggaggatggatgagagattcctattgaactggcagaatgaatatgaagaatgatcataatttgggcaggtggagaagagtaggaaggcattctaggtaggtggagtgatttggatgaacgcacagaggtaggaaggacagcatggtccagaactctcaagcccatcttggcttgagcaaagagtgtggaatggtggctggttccttctctggaggcctcacatcactgcagtgctcaccactccctgtgctgataaataggctgactctgctgacatgcttttaggggctttaaagctctaaggagattgaataagagtccagggtggatagggtgagggaaggaggacaattctatttctatttaaaggagaaaatgaaaatattgtgatgtcatatgtcagaactcaatatattgagagtaaattggtttagagattacctaaatctttgaagaactcacagtgaaaaatctatatgaatatggatataataaccagatatgggatccccccaaaatgaacttaggaattctggtattttacggaatattggaggaatgtaatttatagatagagtagtctattgtacttttaaatctatactagagctgatacctctcccagagtaattgtggacaagctcttcttctcttcccttgggaacatgaaacccaaaaagcccgacttgataacttaaatagcagcattagagctttcttgataaaaatcaattcccataagcatagtggatctcctgcatgaagcagactctcgaaacccaggggactcattttcgtctgccttctcacacttacgctatggtaagaatgaatccctgctaaaaaaccaagacctactgacattgattgacaaaagacagtatgagatgttgataagtggtcaatctgaatagcatcaaagtgaaataaaacaatatattaaactcatatataagagacaggaggtgtttggacaagagagtatctgggcataattttctggtgatttggagatgagctagcaatagcaaaacatactgcaatgttagtcaattcaagggagaaggataaatctgataaacccacctttgattcttccggaagcagactctgaggttgaggttagcatggaagagtttatcagagaatcgctatatgtagaagggaagaaaaggaaacaggattgacgagagggagacgctggactgtaatgcagtctgaacagaggcctcaaagggagctctagagctagggtggctcttcagactagtcccatgttagggtgaggggactgggcctttaaacctctgaatccatttgtcattggatgcagacttcctgcgggggttgggaagaaggaggcatgacttcagggatggtacctcttttccacctcgggcagctcatcactgtccactacagcaccagtggaaaaaatatgtagccatcttagcaagagaaatgtttactattcaactgattattaagacataggattcaataacactaacactaatatcaataactagtatttaacaggggtttattatgtgtaagtaccatgctatatgataatatactgtttcatttagttttataactttgtgacataggcattgctatcctacacttcagtgaagaaactgaagatcagagaggttgaattactggcccagggtcacttatggtacagccaggatttatgctcaggactggctccagtgctgtgtgtaaacctttattctctactggagcacacacagtatattacagtgctgaatattgtttgagaagatacctgtccaaggaatacagatttgcattcctacttaatgtgtggtcttataaacaacatttaaacaagtttcatgagttactgtgtaaatgttaacaatgtttagcagtttacaattgcattacttttaaaaggaaaatgagtaatagttaatgctcgattgactattaaaatctttatttcatgacaagaagacctgaagtagtataactaggtacctttataaagctaacaatgcccctggagctccgaccacgagcatacactctttcatggggaaatcccatccattcaaatatttccaaatacactgtcgattgattggtatcataagcagaatattaggctagaataaaataagtagagttttcgataatcaaaagataatgtacatttattgagtctaatcatgaaggtctcttttgatcatgtaacaggtttcctaatctttgggacagaaattacagttgtcttgctggtttgttttcacttctttaggtctctctacatcaatcttgtgggactctgggcaatcctcacatgctcagtgttttgtgggctcgccctatattccaggtaccatgactgtgatccttggacagccaagaaagtgtctgcaccagaccaggttcagtaccatgtctttcttacaggtgtattaataatattcaaaaagcttattagttgagaggaaagagcattcatattcttgtagagaaacggaaagtggacatgccatcatcatcttacatttctataaaactttgttaagaatttattttagcagatatagcaagaatgaagagtactgcatctaaaatgaaaatatggaaataccaggaaaaaatcaagcagagtgttaaataggaatagctcaaggttgggagtaggaatgtgaacacagttttttgttgttgttgttgtttgtttgtttgttttttaaactacacaaaccattgtactatagaacattttgtgggtgtttgttagagcattcatttataagcaatttatcttccaattttttaaatgatggagagatggatccaataagtgaatatttactgagtaccaactatgtggtgagattctgtgccaagagcttaacatgcttcatttaattctcacaaccctgcaatcctgattttacagatgaggaaatgcttctcagagaggttatataacttacccaaggtcacattgctaataagtaagaataactatgagcagttatcaaatacccaccatgggccaggcactaccataatgcttcatataaatttcaacttttaatcttcacagtcaccctctgatgtaggttctatgattatctgcctttcaaagatgagagaactcaggcctagagaagttaagtgaataactagctagtaacctggagccaggatttaaaaccaagcaagctgccaccagagtcctaactttgaacctctgtgccatttattgtctttcaaaaaggggaactagaattcaaacacaggattgcccaaccctaaagcctgagttcttgccaaaattattttctaagactcacttgcagaacaagttcctaggggattcattagaatgaaaatagattgaatttctgttgatgcaaatgcatctcatgctcccagaaaaatacaacttggtgggccgagaaaataaaaacaccctggagctgtttctcaaccctatcttaacttatgccctccttttgataaacacaccctctctgttctctcagagaaggtaagaatcacgtttactatgaagatggaggcacacttctgttacatcccctctaataaagaatattttgtgactgttatcaatctgtattgtctgtagtttgtatcatgaagacaataacgactttaaaaaaaagtttgtaatatactttgcctttaccctgggccaaaaaaaaaaaaaaaaaaaatccctacggcttcctacctttgagacatcttgtagaatacattcagggtgtcttgcttgcatacgcttagagagtccgtgaagatttctccccaactgatatatttccaggactgtgttagttaacaagttaattcaattaacacttcactggggataccatgcaaataagacagtggaagatcgtgtcaaaaccttatcctcgctcagtcgcggtggctcacgcctgtaatcttaacacttgggaaggctgaggcaggcaaatcacttgaggtcaggagttcgagaccagcctggtcaacatggtaaaatccgtctctattaaaaatacaaaaattagctgggcgtggtggtgggctcccataatcccagctacacaggaggctgaggcaggagaattgcttgaacctgggaggtggagattgcagtgagccgagatcgagccactgcactccaacctgggtgagagagggagactgccttaaaacaaacaaacaaaaaatacaaaaaaaccccttatcctcaagagagcaaggatatgtttctctatgctctgctggcatttgcccagaggaagcaggccagtttctagatatagttttactattttcctctccatcaatcctatttcattggttctacctatacttgagggggccactcagatacgaaataattacaccacagatttttgagaaagttgaagataggatagaggattttggacctttttataacactatagatggaacctacttttgctatttgtgggacattgtttaaagttaaacctttatgatatcttggaaaaattgggtcttgatctcatatagggataaatgctattccgtttttctgagaataaagattgagtaagctttggaaaagtggagaacacagtcctaaaagaactgaaacaatcctaatgttgaaacatttcttttcaacagtggaggaagttcttccatatcccatgagcacactattgttaaatgaaattgaagaggctatggaagccgataaaataggacatgtcatctactctgtactgtgggagaagtaatcaataaggttttagtgcaaatgagaggacactcttcgagaaaattgtccacttaggactcttttgattcggaaactgattttgtagaaaaactgccatgcaacagagtcctgaagtcacacacttgattatcctaatttgatatttattttttaaaatataagttatcaaaaagcaagttagtatcaggaagttttttaacagaagcaagtttaagggatttcctgaagtcattctccacccatcattatgtctcgtacctgatgcacctaaaattacatcttctgtcctgggcagtgactgaagttcacaaaatggccttgagtcatcaagtaaagttaagtggatgctgcttacttaagcacagaggtgtgtcaaatatttccttaaagacaacttattattctcaagaatagcaactactttgttttgagcatttattatattccatatactatactatgagttttacataaattatctaaattaacttttacagcaactctatgaggttagtattattacaactattttatagatgaggaaactgaggctcagaacttcagttacaaaagccgtatctgtgtgaatccaaagcctctttacttaactactgtgctattcgtttctctaagtgttagtagttaaacagtttaattttaggtatttgaaaatttcatttgtgtggaataactcctttcagttcccgaaggagacaagacaaatgataaactaggcattcattaattttattcagtagcgaaagtacttggaaataaattttggaatttttcagctcatgccttatttggtactggacattctgcaagattatccaggacttcctggactttttgtggcctgtgcttacagtgggacattaaggtatgaactatgactctaataacatatgatttccctgttgggatctttcttttattattagatactttagcgggataatgtggagttctgggcacagacacagatggcccaaagtaggcaatctgctgtatgtgtcctctctgtggtctgacttatctctgtggactagcgtcctgttgtcatcggctccacttttaactaggtccctgctctctaatacaccaccatacatcctatgatgtgatacctatggtcacaataatgacgatcggttgatggtagctttcactggtgatcaaaaatgggtgccacagtcttgattgaaaacacacatggggctgaagcgtggtcaactggaaaattagaatgaaatcttccatttacatgttgaataatatatactgcccaaagaatcttacattttgtgatctatcattgccctttctccttgcgtttgttccagagaattgttattatcaacatgtacagtgtgtgttagtggggattcaggaaattaatattgttgatatttacagcacatggtagggaggacttatgacagtcctttctatgcacaaagaaaaatacattttaaagttgttatgcataggaatacggagtaatctatgtagactcttttagacactgaggattaacagcaagtggaagcaacatgaacatatcctttctcttttactgtcaagcctgtagatattgcctgaatatcatttttggatgataacagtttcaagaaagacagtgctgtgattattaaaaatagcatagtagagcctggtgtagtggctcacgcttgtaatcccagcactttgggaggtcaaggcgggtggatcatgaggtcaggagatcgagaccatcctggctaacacagtgaaaccctgtctctactaaaaatacaaaaaattagccagacttgttggcgggcgcctgtagtcccagctacttgggaggctgaggcaggagaatggcgtgaacccgggaggcagagcttgcagtgagtcgagatcatgccattgcactccagcctgggcgacagagcgagactccgtctcaaaaaaaaaaaaaaaaaaaaaaaaaaaaaaagcatagtagaaatcaggttatattttagaagtgacaatttgtttccccctttctcattcccacttaaagtattattaaaggtaaaatattataaatcaaaagttttattttcccttacagcacagtgtcctccagtattaatgccttagcagcagtaactgtggaagatctaatcaaaccttacttcagatcgctctcagaaaggtctctgtcttggatttcccaaggaatgagtaagtttctgttttcataattccattttagctcagagagatgattttttgagacacaaaaatttcttttccactgaagctacagaggaaggacctctgaatatgactggatatccacatatgtatgcctatgacaatgcagatttttaaaaaatgtttgtatcaatgtttaatgttaccatatctgtaatcaagatttgggagacacttcaaacaattaattgtcagtgaaccacaaaggacaatttccaggggaccgatttagctgtccttttttcctgtgccttcatcctatcctaaatttgtgttaaaattcttagccacacacacaaaaaattcaactattttccttttcaactgtagtccacagttctaagaaatatccctagtttgtaaccaagagccacactttttctgttacctaagaaggcactgtcagtttcagttatgttgtcttcccataaacacttcccaaatgtttacagaggattagattaaattagattaatagattagatgaattgaagataaagaaacagagtgttatgaattttgactcgtcttagttgtctgttctgtctgcttatgtacactgtcctggagatgaactataaatttgtgcaagaaattctcaacttctgttctgttcaatcgtagagcctcattaggggttaaataccagcttgaatagagtggtttaagcatcttcagttcccagatgtctcaaatgtaatatccaactcaaagaaatcttgccaatgtactgcattcttcattttgacaccttgaaatgcatgactaacaaattccttttcgagaaagaatctttaacctcaacacaataacatcaatagactgtcaaagaaatagtaaattataccccattagtagccataattctatgtaaaattgccacaactgtagcctggaatgagtccttaatttatatccttcagtattccctaaatttaaatagcaatgcatcattttattatgtacccaattttaatcctggaacataatttcagaatgatgcaatgcttcctaaagggtttttagactggttcattatgggattattgaagctgtgtggtttagtagaatgaatagatgaatagatgcttttgaagccagacaaacccagtcttgctttgttaatttactagctgttaaccttgagaaaatccagttacccatacaagcctctatttctctatataggaaatgggcacacaaatactttgcagaattgggttttgagataatgcatataaaacgggaggcactggctggtgctcatggaatgatgttgatgaatgacaaatcaactctgctaatatagacgagactttcattcattttaaagggtactatgaaatcacaatcttgaagtatatgcagtaaggacatagagaagaatgtggcagatgatttgaatgtgtttccaataactttgaattcccaaaattataatggtgactattcataacctatcaaatggagaatcaaaccaaatatattgaaaatattatttacattaaaattgagaggcgtaaaaatcatagcaatgagaactcttcaaaaaaatcaatttaaaatttttcattcagatgcattatttactcaatacgtttaacaaaggtatgaatgctaaaataaaatagaaataacataaggaatacaaaaattttatgatggacaaatttcccattcattttttttctgtatattgagttttgcatttgggaaattatatcaactgatttcaggtgacttttgctgaatggaagtattaaaacaaagggggtttttttgcatgatatttcctataatttaaaaggtaatacatgtacctggagaaaatttagaaaatactgaaagtcaatagaaaaaaatattccacaatctgaccaccagtggtggtttgaaaacttttataaagaagtttgggctgagtgtggtggctcacacctgtaatcctagcactttgggaggccaaggtgggaggatcacttgagcccaggagtttgaggccagcctgggcaacatagcgacaccccgtctctatgaaaaaaaaatttttttaaagaagtctggggaaaacaacttagcattagggcagatgtgctacttatccagaagttgcctttctttgctagtttaataggaagggcttgaggatactgatggagattatgagggggctaaaagtcgtccaacaccccatagtgtccattgccacttcccaagggaaatgaatgcttaaagtcagaagagtctaatttctgtttattactccttctctcaccttgtacagagcagagctgaatagtattctatttttggcaagctgaaaacagagacctgagcctttctttatatacaaatgtttatggatgattagattaataacacaatatagttcttagttttaaatacctatagtttattccaggaactctttacttatataacctactgttgtaactaatcctgggacacaatgtaagggcttcgtcctcttgaaacactgctgatcctagaggaaaatagccatttcctttattcactggctctgatgtgtgtggccattcttcaccacagtcatattatccactttgaatcaaaggtgtggtggattattctattgagaattctaattctctgggtgtggattttacactggcttttatgttgtccatttaggtgtggtgtatggagccctgtgtattggaatggctgcgctggcgtcacttatgggagctttgttgcaggtgagagctggcccctggaggtttaagtcataaatcactaaatcttttttcaatgttgatgtgaccatccttccagacttctctcgatatatatcgacacctggacatatcaagtggcagggatgactacactttttaattttttttaattaaactttgtgttttgagataattgtggatttacatgcaattgtgagatataatacagagagatctcatatactctttactcagtttccctcagtggtaacatcttgcagtggtaacatcttgatagtacaatatcaaactcatatattgacattgatatagccaagatacaaaacatttctatcactacaagaatccttgctgttgcccatttgtagccacaaccacttcccttctgcccctactccctccttaatccctggcaacaactaatctgttttccatttctataattttaccaggtcaagaatgctacatacatggaattacatagaatgtaacctttttcacttggcataattccctggagattcatccaggttgttgcgtatgtcaataatctgtcctgttttattatcagatagtattctctggtagggatgtatcacagtttgtttacctactcagctgatgaaggacatctaaattgtttccagtttttgagtattacaaacaaatctgttacaaacattacataaaggtttttgtgtgagcataagtcttcatttccctgggataactacccaggagtgcaactgtcaggtgactgctaaatgtctacttttaaaagaaactgccaaactattttccagagcatgtcatttttatatcactagcatagacaaatggcccagtttaaacctcattctttccagcatttagtggtgtctttttttttatattagccattctgataggcatatagtgatatctcattgtagtgttaatttgcatttccctaatggctaatgatgttgaaaatgtttttcagcgacttatttttcatctatgtatcttctttcatacattatctcataatgtcttttgctcatgttctaattcaattgtttgctttttttactggtgagttttgagtgttctttatgtattctgtatactagctcttggtcagatgtggtttacaaatattttcttacactgtagtttgtctttttatcctcataacagggtctgtcaaagtgcatttttttttttttagtttggataaagtctagtttatcaatttgtcctttcatggattgtgtttctggtgtaaagtctaagaactttacctagccccagcttttgaagattttcttctatgtttcttttcaaagagttttagagttttacattttatatttaagtctacaatccctttggagttaattttgtataaaatgtgagacttaggttgacattctcttttcctctatggatgtgcaaccagcaccatttgttgaaaaggctttcttccattgacctgcctttacaccttcgtaaaacgtccattaggcatatttgtgtgagtctatttctgaattctctgttttctttcatttatttatgtgtctgtacttctgccaataccacacagctttataatttgattattttgattactgcagctttaaaataagtttcaagatcaggtcgatcgattcctcccactgtattcttatttttcgaaattgttttagctattctagttcttttgcctttccatatgaagtctaggataatcttgtctgtatctacaaaaaaaatcttgcttaaatattgatagcctgaaagctttttatccatttgagaagaaatgacatctttaccatgttgaattttctaaaacatgaacatggtatgtctcttcatttatttagcttttctatgcaaatcgtattttttatgttgatgtctgtgtgttcaatgctaaaatgtagaaataaaattgatgtgtttatatttatcttcaatcttgtgaccttgctgagctcacttattagttctgataattttttgcttctttgttttatggtttagtttgttttgttatattccttgagatattctacataaacagtcatgtcatcttcgaatggggcagttttatttctttccttctgatctgtatgaatgccttttatttccttattgcactggcttcaacttccatatcatgttgaatagaagtagtgagagtggaaatccttacccagttccccaatgtgaacaggaaactctctattcctattctctattcctatttttttctgagagttttcaccataaatggcagttgaattttttcaaatgctttttctgtaatcaatttatatgatcatgtgatcttcttctttagcctgcttacaggatggattacattgattggttttttaatgcagaaccagccttgcataccgggaataaaccttgtttggtcatggtgtgtagttatttttatatattgctgaattatatgtgctaatattttattaagaatttttacatctatgttcatgaaggatattgatctgtagtggtgtgtgtgtgtgcatgcatgcgtgtgtgtgtgtgtgtgtgtgtgtgtgtgtgtatactgcctttggttttgatagcagggttatactagcttcataaaataaattgggaaggattctgttttctattttctaggagagattgtctaaaattagtgctaattcttccttaattatttggtagaattctctagggaaagcatctgggcctggatttttttttagtttcaaaattatgaatttaatttccttaatagttacagagctattcaaattatccatttcatattcgatgaattgtgacaatttatgtttttgaggaattgttccattttatctaagtttgcaaatttatacatgtatagttgttcatagtagttctgtactatcctttggcatctgcaggacctgtagtgatagcccctgttccattcctaatattggtaatttgtatcttatttttttcagtcttgctacaggtttgtcaatttcattgatgttttcaaagaaccagcttctttttttcattgatttttctgtgttgtttttctgttttcactcattgatttttaacttttatctttatgatttcttttctccttgctttgagttttaatttgctgttttcgaggtgggatcttagattactgatttgaatcttctcctcttttctaatgtgtgcatttattgctgaaaatttccttcttggcaccagtttagctgtgttccacaacttttgacatcttgtatgtttgtttacagtcagctgaatgtatttttacgttttctttgagatattcttttgatttatggattatttagaagtatggtacttagtttgcaagtattcaaagattttcctgttatctttctgttattgattactagtttgattctattgtgatcagagaacacactctttatgatttcagtacttttaaacttgttgaggtctgttttataacctaggatatggtattttggtatatgttccatgagcacttgaaaagaatgtgtattctttttttattaggtagagtgttccataaatatcaattacattctgtttgttgatggttgctgattttctgtttggttgttttatcaattgttgagagaggggtggtgaattcttcaactataattgtgtatttgtctatttcccttttagttctatcagtttttgctttgcttcatgcattgggtcttaatgaaggattaacctttttgttattatataatgtgcctatctgtctctgagaaatttttttgctgtgaggtgtatttattttatattaatatagttacttctactttccattgattaatggttgcatagtatatttttccattttttcactttgaaccacctatgttgctatattagaagtgagtttttttgtagacagcacacagttgagtcttttttaaaaatccatcctgtcaatctctgtcttttcattggtgtacttagaccattcacatttaatataattattggtatgttatgttagggcttaagaatcagtttttagttgtctgtttggtatttctgttttttgttttctgccttcctgtgagttgttctgtggttccttctttctaccatttcctttctgtttagaaaacttcttttagccattcttttagtgttccttcacctgagaatgtcttgagtttcccctttatccccaaaggatatttttgttgggtatatgattctgagttgctagtattttttccccaggagttaaaaaaatatttttctactttctttttgtctctatcatttctgatgataaatctgctactattctaattgttttcccttacaggtaaggagtcatttctttctgaatgctctcaggtttttttgttttgttttgttttgttttttctttagttttcagaagtttaattatgatgtatcttggtgtgtatttccttgttttgagtttttgatgtgtcttggcacgtgtttctttcttttcctatgtttttagggttactcagcttcttgaatcttgaatcagcttctttcataagtctgaaagagatttatgtctcttatcaaatttgagaagttcagacattaattctttgagtacttttccaccccatcttctttctcttttcctctgggactctcatgacatgaatgttaaatatttttgttatagttctacagatccctgaagtttattttatatttcttcatttcatttctctctattgttcagtttgggttatttctgttgttctgtcattcagttcaccgatctttttctctgttccttctatactgctattgtgcccattcatttaaaattttttatttcagttattgtatttgttcagagtaacagtttcatttggttcttcctaatatcttctttttttccctgagatttttctctctatatatattttttccctctattttttcattagtttcaagtgaattctaattgttaaagcattttttaaatcatggctgttttaaattttttatcaaataattctgtttctgtaatctttatatttagtttgagatcttcctggttctttctatgaaaagttaatttctgttgaaacctgggcatttcaatattatgtgatgaaactttgaatcttaccttctgttttagccagctttctctgacactacgtcagtagcataagggagggtgccgcctcattacttcaggtggaggtagaagtccagagggaggagctccttgttatgctggataggggtgtgtagaatactatgagacatattgtggctataagattaatgatagtgcatgaggcccactgaaatatcttgcagggctgatactacatgtcatgtaggaatttacaaccctggctcagggatttccaggaaaaaaaagccacctcagcacagaagcagctttcataaaccttagaacaaagcttacttttacaataatagcttaaataccctttatgaaagaaacagctggtaactaacctggactaaatacaggtataagaaagggagaaggacccccaaagtctgacaatggtctctggatgaagactctctggtcagttcatgatctgaccccctgactgtatctggcccatgacaccagcttattctcactatccatcttctaagagtgctgccagaataaaccgattgagcattagatggtgcctaagactcatctttgatgtgaagtgaacagaaaaggagacatcgcccctggggaagctggttaactaggtccacctacaacctctgaacacaactggcattgaggataggataagtaagtgagcaagtaagtaatggcacccatttctaaggaaggactgggaaagggtgactggggactggttctgatcaagaagtcaaatgaagccctccggaacactccgtagtgtgtctggtagtttttattttactttgtggcctgttgctgccttctacatactttgtctgtgcctctgttgtgaaattgccaccatgaaaaacctgccaaaattattcttaagaccactctggtccccagtaactggggacaggctctaaggaggaggaagcagaggtggaggatgccccggcttgaagagctaaccactggctggtgtgtctctggctcctgctgtgggagggttgctccttgcccccaaggatcttggcactgtgatgccacagaccaagaaggcaaaggtgaagcttacctgctgaagggttatgtgctgttgcccaccagctagggacttagaagcatatttcccaatagatccttggactaacaccaccagtgaagtccacaagaatcatcaaagggcatgtcgcagctgttcccagcccaccatgcactccagctgagacagtgtggcttctcaaggggtgtgccccagcctgtgatgtctgctgagtgttgctggcagcagcccataggcaacatgcccaagtcacgcgggctcacctcagagtcgtgtcattctgcctggtcgatccagagccctgtgagctgtggaatgatgatcctgggtgtgctggcttaccgctggccacaccacaggtgtcaccagtcatcacttgggctgagaaagctgctgccacctccacagctggctgagatgaggaagtgtgtcctagacaccatatggtccatgaagaggttcacccctaatgtggacagaaattcaaagcctgctcaaagacctggtgcaggaacccaaagagaaaggcaccacctggctgtgctgtgtgtacctgagaggaagtgctttgcaactcataggccgagaaatacaaatacagcagccagcggctatctttaaagataataagatcatttccttatttcaagaccctgttgagcttatccaggcacagagtagagtgggtagacataactgtccctgcagtccgctgcagtggctattcaggcctggcgaatagcacccccagaatgagcctcttttcatcaagggctgaaggtggctggtgcacactgaggaagaaggggagccactgtggcgcttaggcatgctccagtgagtttgcacagccgccactcccatgggacaggggcacaactgggagaccttggaacagaccaccttgcacccggatggtttacagatgtttttaaggagagcccatcgtaagccaaaccaactctcctggccctgatggccacagtccctgaagttaattgagctctgtaggtcctcatcaccctggaaaatgctgaagaatagctaagaatccctaagaaaacttagggaactcaccctataccactcttcaggtccagggtccctagcgggctgccttctttgcacctttcagacagaaccatccaacagtgttcacaacagcacctcgctccctacccaccacacaggaccttgcagccagaggtctcacactgggctctgctctccctggccttctcacacccatcacccacgggatgaggcccagtccagctgttcacaagggcagagtgacctctactggcagatacgctgaagggtgcttccaaattggctcctgtctggcaagcctcatgaattcctgcaaactctccttaataccaatgtcgtccagtcatcccacccactgacccaccacccatctggatggctgagcatcaacttccacctttgggaagatgtcttcactgctaacattcctgccctgtgtccaggatgacccacctggactgtcccacccaccctacctatatccctccctggcaagacctttactggtatatttgcctaggggtctcttgggccaccactgttcctgcagccagtgcctcctagagacacttggtgccccagctgtgcctgacagccatctggaatggacaacaccttgaactttggcttcatcgggccgtgttcaagatcacgattttggatctttgacagcacctcagcacacacagtgtccccagacctaattgcttagttctgtgtcctccttatccttctgttccattttcacctgtcattgctctgtaatcaagcaactctaagcctgctaccactgagtccattcaactcctttcacctgctcatgtaatcgtgtttccaagccccagtgtgcccgtgttcttttgctgctactccgggtggcctgactttccacatccaactgtggcttctccagacctattcaccaattacttggcactgaggtcactgacagcctccaccacattttggccacccactgagcagctaaccttccagcctgctgggcatgccttcattggcctgccacacatccattgcttcccgtccagacttggtcgctacctgtcctccactcttcccccatcattggcgagtctgccccaaagccacagccgaaccccattctcatgatctcccgtaactgctcagctggacactagatctactgtatcctctctgtgatgatacagttcatttccaggtttttccaagggcgccaccaactcctactcaccgtcctacagaagatccatttctcaagatttgactgaccatggagcgcactttcaaacttgggcagcctctcaattattgaccccctcagggtcaggggtggagtgtggtatacaatgagatatattatggctataagattaatgataggcataaggccactcaagcaactcagagggtcagtcctatctgtcaaacttagcagtatgaagcaacctggccggggatttccaaccagatttccaggagaccaggtcacctcagcacgatgcaattttcacaaaccttggaacaaagcttacccttacaagcatagcttaatctctctttgtgaacaaaacacctggtaactgacctggattgaatacaagtataagaaaggaggaaggatcccctaaactctgagaaaggtctctagatgaaaaccctcctggtctgtcagtcatctaacctgtgactaaatctggcccacgacaccatcctgctcctgctattcttctggtaagagcactgccagaataaaatgcatgagcatcagacggtgtacaagactcaacaatgatgcaaagcgaactcaaaggaagaggcttccctgggaagctggttaactaggaccacccgaaacacgcgagcaccacagggtggaattcactgacatcatggaggaggtggctttgtcactgctgggccatggtgaaagtcccgattctccactaggcctcccatgtcattgccagcagggaggcaaagggtgcctggtgacagtctggggggatggaatctaggctggcatgggtgtgggtggggtgaacagattattctgtggtgttcggatggaatggagcggttggggtctaagagttttctatcttcctaggctgctcctttcctggccctgtgtctagagagagaacaggattttgtgagggctttttatctttttaaagtttttttgcctgtgtctgttgccatttccaggctggctggcttcttcagttctcaggctgggatacatgaagcaaaaagaaaacttagggaactcaccctgtaccattcttcaggtccagggtccctagcaggctgccttctttgcacctttcagagtcttcttatgtttattttatatggaatgtcaggatttttaattttattagtaggaggaatagagaaacatatgtttatcttctcagaagtagaaatacttcattaaaaaaaatttgtttatttacctgaagggtctttcacctcctgtggcgtttgctcttctgattagagagcatttttgtgttggaaagctccctcctgttcctgtggagctgtgggcctggagaggacagaccaatctttacaacagtgtcttcttcctgaaccctgtgggcacctcactgtcttcaacacgcaggccttggcatagacttctccctctgcctgcaacaccttcctctctacctcgtccactgcctaagccttagttattcttcagttttcattttaacacattacttatttaaagagacttttcctattacctggatacatttgcattcccttctgctaaatgtttctagaagcaccttgtactttctttatcatctccattatcaaactgtattacagttatttatttagtgtctgttttactgaatagcctgtaattcatgaaggtggggaagatgacttcgttgctcaatcctgtatcaaatatctggttcctactgcagtgcccaggacaaagcaagacattaacaactttagcttgaatattaactaataatgtagttaggctcataaatatcctattatctgatgaccccaagtgctccctgaaatttccttggccacttgggctgctcctctgggatcctggacctccccttgacctagaaacttaaaggctagtactggcacagcaggggtcctccaggatctttcttcagtcctaaggcttggggccattctggttggttgttgtttaggacatagcagttgtaaaatagtttgaccccatccctgattatatgaatgaatgaattagatccagaagctccaagcctgcccagcactcctaaaccacagccagtcatacagaatttgctttcttcatagtctgcattagtgcctggagctcttattgtggttgattcccctttttccatatatcagctatggcctgattatatacaatgacatgttctagcaaagtcatcataactccaagccagttcatccttgcttcatctctagccaaggtcttatttcctcttgtaatcttttttgggtgtctttgttatgtctccaaaactgctcacagatcacacagtcatcacttgctttgtgggtcatatgacgtcttgcagtgaagtctatctcccttctgactggactgtgtgtaaactgactcaggatcacatccttgagtccgcatccttttaaacactagttctcctctctactcagtaccacatccttttcttcttccatgaagtaggcctatttaaaacaacaacgacaacaagaagcactatttcctctaatatttccctgacttggtattttctctttgattcattcattctgctctattaaacccctttactctctggacaaaccagtgattccctgctcttcttttatggttgtgtccattcttaagaactgggcattagattattgggaacgtatatctcttggaagcttcatttagttgctttgcctcatgcgatgtttgcactgtaaatcttttttactatcatggatttgatatctctacatcaaagcaaaggttgcagttatctaattagattttaaatctatttctacatgatttgatcagtctttttaaaatcgtatttctcattctagtttcatccttagggaatcaaatcaggaatggcaaatgggtttcatcctcctctctggtcacatagcagtactcgtccaccgttatgagaggattttgagattatcaaagcacaaaggagtgctgtgatttataaacacctgcaagagcaatgctaagaggagaggtatcattcatgatttatacattcacataggcactaccattgtcgcagacctctctatcttctttgtgttgtgtggccttttttgaggctacttgcagaaacagatggatccttgagactgagatgcagaaacttgtaagttctaatgacttcttttccagtgataaggctatcatgactgaaactatgattttcagaaggaggccgaatactttaagtcattatcctgatgaaatgactttgaaatatttgagtttcgatttgagattgctaattgctgacgttgtattatttttgtaggcagcactcagcgtatttggtatggttggtggaccacttatgggcctgttcgctttgggcattttggttccctttgccaactcaattgtaagtacaaagaatgaatatgcttgaggattactttttgaactatactagcagctctacacttttttctcagttggttcctttgagatttgtcattagctacttgtcttgatgacttaaattatttctgttgactttggtggagtgtacaaggaagtactatgtatggggaccctaaatgtgtgaagctcaatggaaacttccagaaccatatagggcaattttaaatattcataatataactaaaggagcaacatttttatgcacgtacctacttgcccttctcaaaaattaatagctaggatttaaaagagatcaataggctcagagatgagagatttaagggcagaaaacttaggatttctgtgaatagccatagcacagcaaagagaaggaacacaatcttaccaccttggccaggaattttctactcctgacatttaaagctgtagctcccacaacatgatttagccccaaaggggtgatcataattgggaatattttccagaaagacgtaatatctctgtccttctacctagacatatctgtgcttagaagtgctaacttttgtttggaataaatgagctagaaattatttctctgaaacccagaagaaagtccactggttcagtctggctataagatgtagttcaggaaaaactgataatgtatgtgcagtcgcttcagatatagaatagccatagaactctgacttcacatttggaattctattttccttataaggcattaggaattggtaaggcagatattaataaggactattgtggttattatttattatcctcaatgaaatgtcatatgaaaagctgcttttgtaggaattaatcacaatggaagagatttgtttgtgtccacctcagaatgttgaagcttggattagattctttgcatgatgaaatgcattagtttgttaagtattagaaaaatgtatttaaaaatcaacttttgatatttggcatttgttgccagcgtgtatttcctccggaagagctgtagctgactaagctaacatgtccttttctgggggcggtaacggaagaataacatactattcttcctgccataaacagtatcttttttttttttaaaccatgtccaggtttttcaccagcagattatctgccttccttaaaagtggcctttctcactcctttttctctccctgcctcaatacctttcattcatttattcacttgttcatttattcaatagatttttatgggctgagcactatcttgggaggagagagcgttgaatgtttctgcccttgtgagttttataatttcgtgccatgttgaacttggctttctaatattaattttgacatcaaagcaaattgtattatttttcttttaaaggcattcttgttttacagggagcacttgttggtctgatggctggatttgccatttctctatgggttggaattggagctcaaatatatcctccacttcctgagagaacattgccattgcaccttgatatccaaggctgtaacagcacctacaatgagacaaatttgattacaaccacagaaatgccatttactactagtgtttttcaaatatacaatgttcaaaggtattgaattaagttttattacattatactttaaaaaatttacgcaacaagtagagaaccccacttgctttttgtcttgcttacaacactgtgattttgcctaattctgaaatgagtaaaacccatgtggttagctatagtattttctgcagcggtaacaaaaaatgtcatattttcataatttctctagaaatttctgccttgtctacagatctaagaagacttaaatattaatgagaaacttctgttatgtgtaaactctcctaaacaccagctcttagctgcatgaagaattatctttgtcttggaaaaacttttaaaatggaaagcacaattatagaataaatattgcttatatagtcttagaaagatagattttactgaccaaaagctacaattatttaaacatgttaaataactgccatttgttcagttgaagattccaaatctttaaagcattagaagtgattgcagctgtgatcttcatgctacagatttcagtcatgcagtacactttggagcctctaaatgctgaagttgtctgatttaacgtactgaaatagtgggtagaggcatgctttattgtacagtgaatgtgaggtcaagactttctttagtggatatataagtgtccagctttaaagcacaaaccctgtgaatacgttcaaggaatgcaaagatgatgccattgcccctagagtattgccccagtgccagcttatctggaatgacatcaatatagtcatacctttggggtcaagacacggcatacccctcttaaaatggacacactcctgagagaaggaacgtgatcatacatatagccattattagcaatttgtgttctggagagaattcagctatgagaaagctagccagtgggacttccaggggcgttccaggcaggtaagctgtgtatggtagagtggaaaggtagttccagagcctgaggcgtatttgagggaagttttactaggtgaggtgctgtggagagaggggcaaggacatgtggagagcagtctggacaggcatgaaagcactaatgggaggggactgggagagtcatcagagtcagaaagggagaaaagattaattgaaggttaagtgagaagacacaggatgttcttcttgttttatgattttgcttagcgttttcctaattatgtaaatatttgcccttaaaagtctaaggcaaaatggttatttggattcctgaaacaaataaattagctgaagtttattctatgcctggtaccataatagttttatatgttagtttacttatcaaatatttcagctaataaacaagaatttttgagcatggactatgtacccagcactgtgctggccatttatttagtatccatggaagaaaatcttcctctcccagccactgtttcctgccttccagaaaattctaatctagcaggagggaatgtgcaaatatacgcaaaacaataaactcagtaagcatgctaagtataaaaactaagacatagtaaataaaaaatacttgatacatttttgttaaaagagtgagtacaaagttggagtgtaagttagcatgaagacttcagagatatactactacttcaagaatgcataggtttggctagatgaaggggaagatctggaaagtcattcttgtcacagaaaatgaccagaagggaaattggacaagaaaataaattaaaacatgcattgtggtaagtaatattcacaggatatcatgggagccagaaggaggaagtctgcctgactgaagtcaaggaaaccttcttagaggagaattgcatgggctggagttagtcaggtagcaggattgagcacagggacacaatctgtatcgcaaggaaggaggagtgtgtgtcgcagaatgaagaaacgaccggtgcaaaagcatgaaggactgaagaatcagggcatcttaggaaatcatcctagttccatagggctggagcacaagggttgtgagccacccagctgaaggttgaatgtgtgatgctttggttgcctagatacaaattgtttcaattgcctagattcagttttgatttttttaagtttacaatatttgatggttacattggtagcttgctgtggttttaatttgtatttcccttaagattaatgaagtcaaatacattttcatttgtttgttggccatttggatatttacttttgcgacttagttattgaagtctttagtccatctgaaatggattctctgtctttttcttacttacataggagttcatgacatattctacatatgagatatgttttgaaaatgtttctctcccattctatgggttacctttttactcttttaatgatactttttttgtcagtcaaaatttcttaattttaatttatttctgtttctcagttttttcctatatggttagtcctttatgtgtcttgtttaagaaatccttgtctaccctgcaatcataaagatattccactaattttttatttaaaagctttattgttaaccatttccattgaggaagtgtaatccactttggaattgatttctgtgggtggtgtgatcaagattatttttccccatgtggatactcagttaactaaaaaccatttattgaaaagactccccttttgccccattgaatgcactggcgatgaaaacgatgttaaccaagtaactatttttctgtgggtctatttctcaattctctactctgttctattttgtctgtctttggctcaatatcacactctcttaatttcattagcttaactcaatgtgttcccctcctttctgggttcttgtcacctcaagttctcattgttttgtttttgtttttgtttgagacggagtctcagtctgttgcccaggctcgagttcaatgcaacctccacctctgggttcaagtgatttttgtgcctcagcctcccaagtagctgggattacaggcatgtgccaccacacctggacaatttttgtatctttagtagagatggggtttcaccaggttggccagactggtcttgaactcctgacctcaggttatccacctgccttggtctcccaaagtgctgggattataggtgtgtgccaccaccccagccaagttctcattgtcttgatctctctgatagcttcaaaaacctgcctccctccccatgccccaggttttatatattgttctcagcagaatgattgttctaaatcaagcagctatcaaaactgaaagtggaatcctagagcaatcattgtagaaatgatcatagaatgttcatgttactctttcacttaaaacattgcaatgagtgccattgcctgtagagtaaaattccaactcatgcctggtcccacaaaaagccccctgcctatctttcatcttgtaatcactgtcttgctcactgtgcctcagtcagctggtgtttagtctctgttttcctcaagaacattttgtttctcagatctcagttcaaatgtcatttattcccaaaggttttctctgaccacttgtagtgtagttcctccctcttcaccctgttaatagcacatcatcttgttttactttccttacagcactttctactagtggaaattattatatttattaatttgtttattgtatttatctctccattagagcataaactatatgttagcaggaatcttatctactttatttttaaaaaataatttcaacttttattttagatacctgtgcaggtttcttacttgggtaaactcttgcctgtctcccttcaccctctggtagtccccagtgtctattgttcccttctttgtgtccataagtaaccaacatttagctcctacttataagtgagaacatgtggtatttggttttctgtgcctgcattaacttgcttggataatggcctccagctgcatccatgttgctgaaaaggacatgattttgttcttttttcatggctgcatggtattccatggtgtatatgtaccacattttctttatccagtccaccattgatgggcacctaggttgattgcatgtctttgctgttgggaatagtgctgcgatgaacatataagtgcacgtgtctttttggtagaacaatttattttcctttggatgtatacccagtagtgggattgctgggtcaaatggtttatctaccttatttatggctgactgtccaatcacaagagcagtgcctgggacatcatgcagaattgaatgaataaacatgaccttcaagtattttagtatatagtcaaggtgaagttttgctttcaattcctgatctgataagataaaattaggacagcaggttatgacattaaaaacattattaagtgttatattttgaccttgccatgggaatcactggtatatactggtagttgctagtggcattacatgaaatcttaccgttgtgtttttgctctcttaggactccactgatggataactggtattctttatcatatctgtacttcagcactgttggaactttggtaacattattagtggggatacttgtcagtttatcaacaggtaactatctaaacattagtattgttgtatttacctctatatcagtttttactgtatctgttttatagctatttatgttattttactatgatcctattgccaactagtttaattatttcttctgttttactcatgggaaatctgaaggcctagacttgaaagcagaagactcatttctatttgaatcttggttctgccctttaggggctgcatgaacttgggcaagtcacatagctacactgagcctcagctttcttatgtataaaatgaggacaataataatcgacctgcccatctcacaagttgctgtaagacaaaaataagatgcttgaaaagaacttcataactgctatgatgttagggattttacacatatttttataataacccaatatgctgtgatctttacatggtccagtattcattctttgtgaactgtaaagaagcatacacaggttatatctaaacatagtgtggaggcttttttttcagatatttatcattttttaaaaagtgtgttcatttttaaaaaatcaaattgatacacatagtatataaattgctctctgggattgctatagaacggttatcttgggactccattttgtcctcattctatgaattctcttagcccctgttcctgtgttgggttttctactgcctatattcattacttctcattctcatttactcccttaattgaatgaaacacatcctatagcatattctcaagaaagagtagacaaaagaagaggttttggaaactttgtgtgtctgaaaaaaaaattatttaatctatcttatgcttgattgattgtttgagtagaaattgctagggttgaaaagaacttttcttttttctgagaggatgtctgaatttttttctagcttgctatgttgctgttgtgacagcgctctgctaccagctgacactttgtgtgtgtcccattttcacaatgactgccttggcctaggccttcagtagggcccattcaatctgaagatccatgcccttcagttctggacattttcttttttgttttcttttcttcctttttatttctatttttaataattttctgtgttctctctctgagaactcctattagtcaaacatttgaatcagaatcatggacagttcctctaattttctcgtttttttctctcttatttttcctctccgtgtcttttaggtctactatgtgagatattttttcacttacttttccaataattctcaaaggagtgctctgatccaattttgagaatagtctgtaggggaacgagggcagggaagaggctattgcagtaacccaggtgagagatcacagtgctggggccggggtagtgcggagttggtgaaacatgctgggctccctgactccctacatcccctcacatgtatgcatccatagcagccacgccaggggaagggtgcaactgcctccttcaacatgtgttacaactagttgtgttcaaggatgtctgatgtctttgtttgctccatgctacaaagctgccagcactgtgccactttacagaatcctgctatcattttctgttttttggcactgagatagcactgtctctcaaacttctgtgacacttgtatttataattataaaatttgttgctattcattatttctcagttgttggaaaatattctttcttttccaaaaggactgtgctctttctcaaaaataaaattcccagtacagtaggtttattaatttgtttggtccagtttgatatattttcagacagatttttgttgttgttgttgttgctgttgagatggaatctcgctcttttgcccaggctggagtacagtggtgcgatcttggctcactgtaacctctgcctcctgcgttcaagggattcttctgcctcagcttcccgagtagctgggactacaggcacgcaccaccacgcccggctaatttttgtaattttaatagagatggggtttcaccatattggccaggctggtctcgaactcctgacctcaggtgatctgcccaccttggtctcccaaattgtagggattacaggcgtgagccaccaccatgcccagtcttaagacagattttcatcttttcccagcagttattcagttgttcagatctggaatacacctaaccagtctccctgtactatttgccctttggtccttatttagctcattttttaaaagaaagtggagtatcagagtagctcctttgaatactcctttttttcttttaaatgctttgattctgaaaaaccatatacagctatatcttttgtttcaagaagtaacactctacctcagtgactgggatccagtaatagaaaacacttcaacttcattaacttcacaaataacttattcctacttcacagaagcagtgagtaccttgaaaactatgagaagcacacaattttgatgctccctgggaaacagattttaaaacctatcgtagtatactaaaactcccataagagttcaggccttacagacttggttggcgtttcagcactcccctgatttttactatgcagaaggctatgttttcatcaggaaaatgggggcaaatagtttcctaagtagatcacaaactgtgggcacagagattgttttgtttctttgtttctctagcacctagcagcatctccagcacacagtaggtactcaataacattgaacgaattcatttaaaattgattctatctccagaacagcagaggttcccataattaaaagtctagtatttgtactaaagtagtggttcttaaactttagagggcataaggatcctcttggaaattttataaaaatcaggcttcaggggtcctacctgcagaggtcctaatttggtcagtccaggctagagtctgggaatctgcattttaagtacattccttgaatgatttggggaagggtagtttgaggaccactctttgagatacacaatttttaaaagcatcctctttgatccacaaaaaataccaaagcaaaatagaattttttttttttttttttgtaaagaaaaccttagggaagaggatttggatcaaacgtcagtcagcatactaattttcacttaagtaatttattcagcagttccgaattgcctgcatttcttcatagacatgtatatttgtagccaacaaagtggggaaaagcagctccactgtctgaagcggggcagatggtttgatattttactgatggcatggagggtgcttttaaaccagttttcctaccagcattgggccagatgactgtttctctagttgagtaccagatgaagcagttggcttgcgtttaagctctatctcacacacatatatatgatacatatatatatatatatatatatatatatatatatagagagagagagagagagagagagagagagagagagagagagagagagagagagagagagagactcatatttattatagtgagaggctttcaagacctggggctaattaaggaaaggggaattgcgggctaagtgatcagtgcttttaagtttccctttctctttgtgttttttgaatgtatggagttgaacgtgaacaagttaaatgcctgtataatggaatgtctctgtgtagttactggtgtcctttttaacaacgtaagtcatgtacatttttttttccaggaggaagaaaacagaacttagaccccagatatatactaaccaaagaggactttttatccaattttgatatttttaagaaagtgagttggctttcatttaccttgagttaggaaactgggctttattacctggatagaacactaattagttctcaacctctttcttgaaaagtgatggacaaggaaggtatagaccctatataatctgatgatctatatatgttggatagctctctatcctatgatgatctatatctgttttatagctccatatcctctgatacgctatacctattgcttatctgtgtatcctctgatgagctatatctgttgcatatcctctttcctttgatgatctttatctgctgtatatctctctatcctctgataacctatatctgttgtatatccctctattctctgatgagctacacctgttgtatatctatctctctctatcctctgataacctatatctgttgtatatccctctattctctgatgagctatacctgttgtatatctctctatatctgtctatcctctgataatctatatctattgtatctctctctatatctgtctatcctctgataacctgtatctgttgtatatctctctatatctgtctatcctctgataacttatatctgttatatctctctatatatctgtctatcctctgatattataaatctgttgtatatctattgtctgatggactattttgtatctatcttctgataacctgtatctgttgaatatctcggtatcctctgaggaagtatacctgttatatatttctctccctcagtagattagaaagctgatgcagagaaataaaaatagtagaaacaattatttagaattacatgaatgaagagcttctttttctccccctaatcaccatgttaacattttcttttagaagaagcatgttttgagctataaatcacatccagtggaagatggtggaactgataatcctgctttcaaccacattgaattgaactcagatcagagtggcaagagcaatgggactcgtttgtgaagctgctctgatactagatatccttaaatgatgtttcaattttatatgttttctaagataattggatcaggttttctttgtgtgtgtgtgtgtgttgtatcatgagtgtttgggggataagtttttgttaaaacaaagtctggactatcttcatttactacatcattaattgatgttactctggagtttagaattctggcattgacatttccctctctttcctttatttcgatgaagctataattgtgaaaattgtaactacatagatgctgaaaggctaatacacacatatgcacatgtatttgattgtcaaaggtatattcttaaatttgggtattattgaaaatattttccatgccttggtgctagcatataagtttggaagtttgccaacatcacaattcatcttgaaaagagcttttttccctcctaccacatacaccattcttagggagcaatgaggtaacaggtctgtgttgtctagatctttgctttttatccccctatcagtccagggcatatactaacctgcaaactgattctgaatcaggaaggtggtaatcaataagtattctggctgggaaagaccgtgggcccaatgatcaaagtcttcttggtgctgttcattaattcttgtgccttttggcttgttttctagagtttctgggctttggctgctgatactgcctttcttagactgtaatttttatctgcatgcccagtttctgacctatcaacttgggttttattgtgcactctaactgagcttgtcttcataattttctgtttattgccctgggcttggatatgtctcaagacactcatgtgaatcatgccaccccaaatcctggcttatcaagtcccagactataaattatgaactcccattagcttggtactaacatatacttgatgtaggtatttatggacttgatgatccaagaatattatattcttcaaaatggttaagctccatggagttagatgactacacttaatgctattaagttgaacttttgaatgtcaactaatttgcaatcaattaaagatacatatgcctagaaattttgaaatttcggtatatttatccagttaaagggctaaattatataagcaaacactactttttttaaaacgtctggactcaaaaaatgctttgttccatgttttaaaatttttaagtagcagtctcaaagttgcttagctgtttattttgctatgttcctagctaagagtttggttataggagttcatcaataacttattttttgtacagttcccacattagatactgtttaaaagttcttttttaaactcaattttttttagaaacataagagaaatatttagatacatacaaatgtttttatgattaaataattttatgcttattttctgatacgtgttatttaggtaatcatgccctgtacatttagaggttgctaactgacaatgttaagaaatttaaaaaaaaaaaaagcctgggcatgatggctcatgcttgtaatctgaacatttgggaggctgaggcaggaagatcgcttgaggtccagagtttaagtccagcctggaaacatagttagacctcatctctacaaaaataaaaaataaaaataaaaaaaacttagctaggcatgttgccacatacttgtagtcccagttattagggaagctgaggtgggaggatagcttaagcccaggatttcaaggctgcattgagctatgattacaccactgcactccagcctgggtaacagagtgaaatcttgtctctgggaaaaaaaa

SEQ ID NO: 27 - DNA, 2192 bp, Homo sapiens
agattcgctggaagcagctggaggctccagttctcatctgctcaggtgtccccggcgccttggcgaactcggccactccagttcctcacgtggtgagcactcagggcagcgggtcgattttccgaggtcccatacctgggtttgaggggcgcggctcgcagcggcgggtgcaggggcgactgccagccctcaccccgcctcggggtgcgttcggaggccgacacctggaggacgcctccagtccccgcgggacgccacgcctgcgcgccagggatccgggataagaagtgcgcgccgggctccggctgcgcgccgcggggccaccagtttgcgcgcagggctcaggcgaccgtgcggccatggacacgccacggggcatcggcaccttcgtggtgtgggactacgtggtgttcgcgggcatgctggtcatctcggccgccatcggcatctactacgccttcgctgggggcggccagcagacctccaaggacttcctgatgggcggccgcagaatgaccgcagtgcccgtggcgctgtccctcaccgctagcttcatgtcagccgtcactgtcctgggcaccccctccgaggtctaccgttttggggccatttttagcatctttgccttcacctacttctttgtggtggtcatcagcgcggaggtcttcctcccggtgttctacaaactgggaattaccagcacctacgagtatttagaacttcgatttaacaaatgtgttcgtctctgtggaacagtcctcttcattgttcaaacaattctgtatactggaattgttatttatgcccctgccctggctttgaatcaagtcacaggatttgatctgtggggcgcggtagtggcaacgggggtggtctgcacattctactgcacactgggtggtcttaaagcagttatctggacagatgtttttcaaattgggatcatggtggctggatttacatccgtgattatacaggctgtggtgatgcaaggtggaatcagcactattttaaatgatgcctatgatggtggaagattaaatttctggaattttaatcctaaccctttgcaaagacacaccttctggacaattattataggagggaccttcacatggaccagcatctacggtgtcaaccaatcccaggtgcagagatatatttcttgtaaaagcagattccaggcaaaactgtctctctacatcaatcttgtgggactctgggcaatcctcacatgctcagtgttttgtgggctcgccctatattccaggtaccatgactgtgatccttggacagccaagaaagtgtctgcaccagaccagctcatgccttatttggtactggacattctgcaagattatccaggacttcctggactttttgtggcctgtgcttacagtgggacattaagcacagtgtcctccagtattaatgccttagcagcagtaactgtggaagatctaatcaaaccttacttcagatcgctctcagaaaggtctctgtcttggatttcccaaggaatgagtgtggtgtatggagccctgtgtattggaatggctgcgctggcgtcacttatgggagctttgttgcaggcagcactcagcgtatttggaatggttggtggaccacttatgggcctgttcgctttgggcattttggttccctttgccaactcaattggagcacttgttggtctgatggctggatttgccatttctctatgggttggaattggagctcaaatatatcctccacttcctgagagaacattgccattgcaccttgatatccaaggctgtaacagcacctacaatgagacaaatttgattacaaccacagaaatgccatttactactagtgtttttcaaatatacaatgttcaaaggactccactgatggataactggtattctttatcatatctgtacttcagcactgttggaactttggtaacattattagtggggatacttgtcagtttatcaacaggaggaagaaaacagaacttagaccccagatatatactaaccaaagaggactttttatccaattttgatatttttaagaaaaagaagcatgttttgagctataaatcacatccagtggaagatggtggaactgataatcctgctttcaaccacattgaattgaactcagatcagagtggcaagagcaatgggactcgtttgtga

SEQ ID NO: 28 - DNA, 27 bp, Artificial Sequence
  Oligonucleotide
actaagcttatggacacgccacggggc

SEQ ID NO: 29 - DNA, 31 bp, Artificial Sequence
  Oligonucleotide
gccggatcccaaacgagtcccattgctcttg

SEQ ID NO: 30 - DNA, 45 bp, Artificial Sequence
  Oligonucleotide
taagcttggtaccgagctcggcgcgccatggacacgccacggggc

SEQ ID NO: 31 - DNA, 43 bp, Artificial Sequence
  Oligonucleotide
aaatccagccaccatgatcccaacttgaaaaacatctgtccag

SEQ ID NO: 32 - DNA, 43 bp, Artificial Sequence
  Oligonucleotide
ttgggatcatggtggctggatttgcatccgtgattatacaggc

SEQ ID NO: 33 - DNA, 36 bp, Artificial Sequence
  Oligonucleotide
ccaccaaccataccaaatacgctgagtgctgcctgc

SEQ ID NO: 34 - DNA, 30 bp, Artificial Sequence
  Oligonucleotide
gcgtatttggtatggttggtggaccactta

SEQ ID NO: 35 - DNA, 47 bp, Artificial Sequence
  Oligonucleotide
tttaaacgggccctctagactcgagctacttgtcatcgtcatccttg

SEQ ID NO: 36 - DNA, 38 bp, Artificial Sequence
  Oligonucleotide
ctacaatgagacaaatttgatgacaaccacagaaatgc

SEQ ID NO: 37 - DNA, 38 bp, Artificial Sequence
  Oligonucleotide
gcatttctgtggttgtcatcaaatttgtctcattgtag

SEQ ID NO: 38 - DNA, 32 bp, Artificial Sequence
  Oligonucleotide
ttaggtacccatggaggtgaagaactttgcag

SEQ ID NO: 39 - DNA, 27 bp, Artificial Sequence
  Oligonucleotide
ccgggatccgtagaaatgggtagtctc

SEQ ID NO: 40 - DNA, 47 bp, Artificial Sequence
  Oligonucleotide
taagcttggtaccgagctcggcgcgccatggaggtgaagaactttgc

SEQ ID NO: 41 - DNA, 47 bp, Artificial Sequence; identical to SEQ ID NO: 35
  Oligonucleotide